SEQUENCE LISTING (17 sequences)

SEQ ID NO: 1 - DNA, 21 bp, Artificial Sequence
  his-tag
atgcatcaccatcaccatcac

SEQ ID NO: 2 - DNA, 761 bp, Human
taaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtga

SEQ ID NO: 3 - DNA, 33 bp, Artificial Sequence
  c-myc tag
gccaccatggaacaaaaacttatttctgaagaa

SEQ ID NO: 4 - DNA, 777 bp, Human
tggaagaaggctttgcggatccctggaggccttcgggtagcaactgtgaccttgatgctggcgatgctgagcaccccggtggctgagggcagagactctcccgaggatttcgtgtaccagtttaagggcatgtgctacttcaccaacgggacggagcgcgtgcgtcttgtgaccagatacatctataaccgagaggagtacgcacgcttcgacagcgacgtgggggtgtatcgggcggtgacgccgctggggccgcctgccgccgagtactggaacagccagaaggaagtcctggagaggacccgggcggagttggacacggtgtgcagacacaactaccagttggagctccgcacgaccttgcagcggcgagtggagcccacagtgaccatctccccatccaggacagaggccctcaaccaccacaacctgctggtctgctcagtgacagatttctatccagcccagatcaaagtccggtggtttcggaatgaccaggaggagacaactggcgttgtgtccaccccccttattaggaacggtgactggaccttccagatcctggtgatgctggaaatgactccccagcgtggagacgtctacacctgccacgtggagcaccccagcctccagaaccccatcaccgtggagtggcgggctcagtctgaatctgcccagagcaagatgctgagtggcattggaggcttcgtgctggggctgatcttcctcgggctgggccttattatccatcacaggagtcagaaagggctcctgcac

SEQ ID NO: 5 - DNA, 66 bp, Artificial Sequence
  P2A
ggaagcggagctactaacttcagcctgctgaagcaggctggagacgtggaggagaaccctggacct

SEQ ID NO: 6 - protein, 554 aa, Human
ATMEQKLISEESWKKALRIPGGLRVATVTLMLAMLSTPVAEGRDSPEDFVYQFKGMCYFTNGTERVRLVTRYIYNREEYARFDSDVGVYRAVTPLGPPAAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPITVEWRAQSESAQSKMLSGIGGFVLGLIFLGLGLIIHHRSQKGLLHGSGATNFSLLKQAGDVEENPGPMHHHHHHILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 7 - protein, 242 aa, Staphylococcus aureus
MNKIFRVLTVSLFFFTFLIKNNLAYADVGVINLRNFYANYQPEKLQGVSSGNFSTSHQLEYIDGKYTLYSQFHNEYEAKRLKDHKVDIFGISYSGLCNTKYMYGGITLANQNLDKPRNIPINLWVNGKQNTISTDKVSTQKKEVTAQEIDIKLRKYLQNEYNIYGFNKTKKGQEYGYKSKFNSGFNKGKITFHLNNEPSFTYDLFYTGTGQAESFLKIYNDNKTIDAENFHLDVEISYEKTE

SEQ ID NO: 8 - protein, 240 aa, Staphylococcus aureus
MKKFKYSFILVFILLFNIKDLTYAQGDIGVGNLRNFYTKHDYIDLKGVTDKNLPIANQLEFSTGTNDLISESNNWDEISKFKGKKLDIFGIDYNGPCKSKYMYGGATLSGQYLNSARKIPINLWVNGKHKTISTDKIATNKKLVTAQEIDVKLRRYLQEEYNIYGHNNTGKGKEYGYKSKFYSGFNNGKVLFHLNNEKSFSYDLFYTGDGLPVSFLKIYEDNKIIESEKFHLDVEISYVD

SEQ ID NO: 9 - protein, 150 aa, Staphylococcus aureus
MKKKFLSLLTLTFFSGLALAADYDNTLNSIPSLRIPNIETYTGTIQGKGEVCIRGNKEGKSRGGELYAVLRSTNANADMTLILLCSIRDGWKEVKRSDIDRPLRYEDYYTPGALSWIWEIKNNSSEASDYSLSATVHDDKEDSDVLMKCP

SEQ ID NO: 10 - DNA, 21 bp, Artificial Sequence
  primer
taagggtactatcgcggcgga

SEQ ID NO: 11 - DNA, 21 bp, Artificial Sequence
  primer
cgtgaaattaaccgtcacact

SEQ ID NO: 12 - DNA, 18 bp, Artificial Sequence
  primer
actcgatgataactgggg

SEQ ID NO: 13 - DNA, 20 bp, Artificial Sequence
  primer
cccccagtaatccataaagg

SEQ ID NO: 14 - DNA, 21 bp, Artificial Sequence
  primer
tcatggcagaacagcagtcag

SEQ ID NO: 15 - DNA, 21 bp, Artificial Sequence
  primer
actcatcttaccattaagaag

SEQ ID NO: 16 - DNA, 22 bp, Artificial Sequence
  primer
acgagttgacaaaggtttagcc

SEQ ID NO: 17 - DNA, 21 bp, Artificial Sequence
  primer
gaaccaaccgctaatgcctga

(19) United States
(12) Patent Application Publication
(10) Pub. No.: US 2021/0069314 A1
(43) Pub. Date: Mar. 11, 2021

(54) Compositions and Methods for Treatment of Cancer

(71) Applicants: David S. Terman, Pebble Beach, CA (US); David Bradley, Grand Forks, ND (US)

(72) Inventors: David S. Terman, Pebble Beach, CA (US); David Bradley, Grand Forks, ND (US)

(21) Appl. No.: 16/896,060

(22) Filed: Jun. 8, 2020

Related U.S. Application Data
(60) Provisional application No. 62/859,566, filed on Jun. 10, 2019.

(51) Int. Cl. A61K 39/085; A61P 35/00

(52) U.S. Cl. CPC A61K 39/085; A61K 2039/54; A61P 35/00

(57) ABSTRACT

Amid their enormous biologic diversity, we have discovered a new group of evolutionarily modified SAgs, SEG-SEI, that in partnership with endogenous SEG-SEI retain the ability to generate anti-tumor T effector cell devoid of the cytokine-mediated toxicity. Such toxicity has hampered the effective use of canonical SAgs for human cancer treatment. For their MHCII partner, we selected the human HLA-DQ8 allele and show that its contact with SEG-SEI is obligatory for the anti-tumor effect. Here we further show that SEG-SEI collaborate with HLA-DQ8 alleles to expand, differentiate, and chemotactically recruit a tumor neoantigen-primed tumor reactive T cell population that propagates both an acute tumoricidal response and long-term T-cell memory/survival.

### CROSS REFERENCE TO RELATED DOCUMENTS

The instant application is a continuation in part of US provisional patent application 62/559,566 filed on Jun. 10, 2019 which application is incorporated in entirety by reference in instant application along with its reference. All references cited in the instant application along with their references are incorporated in entirety by reference.

### STATE OF THE ART

The rapidly evolving field of tumor immunotherapeutics has unveiled key oncogenes, cytotoxics, accessory molecules and immune modulators capable of activating and constraining the anti-tumor response and altering the immune balance within the tumor microenvironment (TME) (Zappasodi R et al. Cancer Cell 33.581-598 (2018); Schietinger A et al. Trends Immunol 35, 51-60 (2014); Corrales L et al. Cell Res. 27, 96-105 (2017)). Recently, immunotherapy of cancer has achieved several notable successes using both antigen specific and antigen nonspecific approaches (Rosenberg S A et al. Science 348, 62-68 (2015); Sadelain M. J Clin Invest 125. 3392-3400 (2015); Wei S C et al. Cancer Discov. 8, 1069-1086 (2018)). Several immunotherapeutic agents are now approved for treatment of a broad-spectrum cancer types the most notable of which are T cell agonists anti-PD-1 and anti-CTLA-4 which operate by stimulating T cell inhibitory and co-stimulating pathways respectively (Topalian S L et al. Cancer Cell 27: 450-461 (2015). Of paramount concern to investigators is that a substantial fraction of patients fail to respond to these measures (Hodi F S et al. N Engl J Med. 363,711-723 (2010); Brahmer J R et al. N Engl J Med. 366, 2455-2465 (2012). This has fueled a universal search for conceptually new tools and strategies to eradicate solid tumors. To this end, we examine bacterial superantigens the most powerful T cell agonists known with intrinsic TCR and costimulatory ligands in an effort to harness their vast hidden potential for cancer treatment.

The enterotoxins of Staphylococcus aureus (SEs) comprise a group of globular proteins with diverse sequences. Because they are the most potent T cell mitogens known capable of activating up to 20% of the T cell repertoire, they have been termed superantigens (SAg) (Langford M P et al. Infect. Immun. 22, 62-71 (1978)). SAgs bind MHC class II molecules at a low-affinity site (KD=˜10⁻⁵M) on the α chain and form a zinc dependent, high affinity linkage (KD=˜10⁻⁷ M) on the polymorphic β chain (Fraser J D et al. Immunol Rev. 225:226-4311 (2008)). MHC-bound SAgs further form a bridge between the α- and/or β-chains of MHC II molecules and the variable component of the β-chain of T cell receptors (Vβ-TCRs) leading to T cell activation (Marrack P et al. Science 248,705-711(1990)). This canonical MHCII-SAg-TCR model has been recently expanded following the discovery that SAgs possess intrinsic ligands that engage homodimer sites on B7-2 and CD28 co-stimulatory molecules resulting in potent stimulation of CD4+ T cells (Arad G et al. PLoS Biology. 2011; 9:e1001149; Levy R et al. Proc Natl Acad Sci. 113, E6437-E6446 (2016); Fraser J D. PLoS Biol. 2011 September; 9(9):e1001145. doi: 10.1371/journal.pbio.1001145). While SAgs induce non-specific, polyclonal expansion of CD4+ and CD8+ T cells along with cytokines such as interferon-γ (IFN-γ), they also activate recall T cell responses against viral, bacterial, autoimmune and tumor targets (Shu S J Immunol. 152,1277-1288 (1994); Coppola M A et al. Int. Immunol. 9, 1393-1403 (1997); Torres B A et al., J Immunol. 169:2907-14.2002; Meilleur C E et al. J Infect Dis 2018. doi: 10.1093/infdis/jiy647). Despite these shared properties, individual SAgs exhibit broad functional diversity in the quality and strength of T effector cell, T regulatory cell (Tregs) and cytokine responses largely due to their differential topology and affinity for MHCII haplotypes and TCR (Terman D S et al. Front. Cell. Infect. Microbiol. 3:38-48 (2013); Chintagumpala M M et al. J Immunol. 47, 3876-3881 (1991); Mollick J A et al. J Immunol. 146,463-468 (1991); Nooh M M et al. Immunol 178, 3076-3083 (2007) Kotb M et al. Nat Med. 8, 1398-13404 (2002); Norrby-Teglund et al. Eur J Immunol. 32, 2570-2577 (2002)).

When used to treat cancer in humans SAgs have been unsuccessful due to the presence of pre-existent neutralizing antibodies that inactivate and remove them from the circulation. In a clinical trial with SEA, every patient exhibited elevated baseline levels of neutralizing antibodies against SEA (Alpaugh et al., Clin. Cancer Res. 4: 1403 (1993). Attempts to remove the epitopes in SEA reactive with the neutralizing antibodies did not improve the effectiveness of this agent in patients with significant levels of neutralizing antibodies (Hawkins et al., Clin. Cancer Res. 22:3172-81 (2016)). Further attempts at dosing to achieve SEA levels greater than the those of neutralizing antibodies failed to improve the tumor killing and led to greater toxicity (Cheng J D et al., J. Clin. Oncol. 22:602-9 (2004)).

In humans, unmodified SAgs SEA or SEB have been associated with severe dose-limiting cardiopulmonary toxicity that has nullified their ability to exert a therapeutic effect. While SEA and SEB were used together in mice to induce a tumoricidal effect in mice, each of these agents induced stage 3-4 toxicity in humans or toxic shock in humanized MHCII transgenic mice (Llewelyn M et al., J. Immunol. 172:1719-1726 (2004)); Kominsky et al, Int. J. Cancer: 94: 834-841 (2001); Alpaugh et al., Clin. Cancer Res. 4: 1403-1411 (1993); Young et al., Am. J Med. 75: 278-286 (1983); Taneja V and David C S Immunol. Rev. 169: 67-79 (1999)). With full knowledge of the toxic effect of SEA or SEB as used alone in humans, the skilled person would be dissuaded from using both together to treat human cancer. Attempts to modify this toxicity by eliminating MHCII binding sequences in the SEA molecule led to modestly improved toxicity but conferred no added anti-tumor efficacy (Hawkins, R E et al., Clin. Cancer Res. 22:3172-81 (2016)).

While superantigen induced tumor killing is mediated largely by IFNγ, the generation of cytotoxic T cells, toxicity is ascribed to SAg induction of TNFα which leads to the appearance of the full or partial picture of toxic shock (Miethke J. Exp. Med. 175: 91-98. (1992)). Most superantigens simultaneously activate large quantities of TNFα (Norrby-Teglund et al., Eur. J. Immunol. 32: 2570-2577(2002); Llewelyn et al., J Immunol. 172:1719-1726 (2004); Tilahun et al., Mediators of Inflammation doi.org/10.1155/2014/468285; Rajagopalan et al., Tissue Antigens 71:135-145 (2007)). Toxic effects of TNFα induced in humanized MHCII tg mice by most SAgs surface early after SAg treatment placing a severe limitation on the ability of SAgs to display tumoricidal effects.

The acute lethal toxicity exhibited by superantigens SEB and SPEA in humanized MHC-DQ tg mice due to induction of high levels of TNFα is a powerful disincentive to the use of these or related SAgs to treat cancer. Since SEI induces the highest levels of TNFα among all SEs (Terman et al., Frontiers in Cellular and Infection Microbiology 3: 1 doi: 10.3389/fcimb.2013.00038) its use alone to treat cancer would be counterintuitive. Likewise, SEG's weak ability to induce IFNγ in human T cells would dissuade the skilled scientist from using it to treat cancer (Terman et al., supra 2013)). Hence, based on individual cytokine profiles of SEG and SEI generated by human T cells (Terman et al., supra 2013) it could not be assumed that that SEG and SEI would induce a surge in IFNγ levels in vivo while barely raising TNFα levels.

Wild type SAgs alone or fused to tumor targeted antibodies used in cancer therapy have demonstrated anti-tumor effects in animal models (Dohlsten M et al. Adv Drug Deliv Rev 31,131-142 (1998); Patterson K G, et al. PLoS One. 2014 Apr. 15; 9(4):e95200. doi: 10.1371/journal.pone.0095200). Their efficacy in humans, however, has been hampered by hemodynamic toxicity and the presence of pre-existent sero-reactive neutralizing antibodies (Alpaugh R K et al. Clin Cancer Res 4, 1903-1914 (1998); Hawkins R E et al. Clin Cancer Res 22 3172-3181 (2016); Cheng J D et al. J. Clin. Oncol 22, 602-609 (2004)). Such antibodies nullified the therapeutic effect of SAgs, contributed to their toxicity, and narrowed the number of human cancer patients eligible for treatment (Dohlsten M et al. supra (1998); Alpaugh R K et al. supra (1998). Importantly, the small group of renal cell cancer patients with minimal levels of neutralizing antibodies exhibited long term survival suggesting that SAgs could work effectively in their absence (Hawkins R E et al. supra (2016). While genetic editing of MHCII binding sites on the SEA molecule attenuated the hemodynamic toxicity, deletion of epitopes that bind neutralizing antibodies has met with only limited success (Cheng J D et al. supra (2004); Alpaugh R K et al. supra (1998); Hawkins R E et al. supra (2016)).

The key elements from the state of the art are that SAgs in humans are toxic and their biologic effects are nullified by a high incidence of sero-reactive neutralizing antibodies in humans. Such neutraling antibodies have restricted their application to a very small percentage of human cancer patients. Even removal of the major epitopes on the SEA binding molecule have not solved this problem. Increasing recognition of the variable T cell stimulating and cytokine profiles of SAgs and their dependence on the nature of their MHCII partner. The latter has been shown to govern the strength and quality of SAg T cell and cytokine responses. In this context, the identification of a SAg-MHCII partnership with the ability to generate high levels of T effectors cells and IFNγ with low levels of toxicity-inducing cytokines have not been identified. An excellent model for evaluation of SAg responses has been the MHCII humanized mouse models. These models faithfully reflect human-like SAg clinical and T cell/cytokine responses to a greater degress their wild type murine counterparts. In these models, canonical SEs such as SEB, SpeA and SEC have all shown toxic shock at relatively low doses largely related to massive surge of TNFα, IL-6 and/or IFNγ. Such toxicity would preclude their usage in human cancer treatment. (Welcher et al., J. Infect. Dis. 186:501-10 (2002); Llewelyn M et al., J. Immunol. 172:1719-1726 (2004)); Terman et al., supra (2013); Tilahun A Y Mediators of Inflammation doi.org/10.1155/2014/468285; Rajagopalan G et al. Tissue Antigens 71:135-145 (2007)).

### OBJECTS AND ADVANTAGES

Accordingly, several objects and advantages of this invention are as follows:

The objective of the present application is to bring forth a new SAg-MHC partnership devoid of toxicity and sero-reactive neutralizing antibodies that displays properties of stimulating T effector cells while limiting production of toxicity-producing TNFa. Here we use humanized mice expressing various human MHCII alleles to assess the anti-tumor effect and toxicity of two non-canonical SAgs, SEG-SEI.

The claimed invention remedies concerns producing a vastly improved SEG-SEI partnership devoid of neutralizing antibodies or significant toxicity while preserving significant anti-tumor effects. For this task, we selected superantigens SEG and SEI. The have been shown to be devoid of neutralizing antibodies in human sera (Holtfreter et al., Infect. Immun. 72: 4061-71 (2004)). This has been largely ascribed to weak transcription and suboptimal toxin secretion by the parental Staphylococcus aureus (Holtfreter S et al. Infect Immun 72,4061-4071 (2004); Roetzer A et al Toxins (Basel) 8, E314-E322 (2016)). Over time, these SEs have also undergone evolutionary structural changes that have modulated the quality and strength of their superantigenic T cell stimulation and cytokine output and distinguished them from canonical SAgs. Hence, the claimed method overcomes the problem of neutralizing antibodies

The claimed invention using SEG and SEI overcomes the toxicity of previously used SAgs while retaining potent tumoricidal effects. This was discovered while using humanized transgenic MHC-DQ8 mice to test the anti-tumor effects of SEG and SEI. Surprisingly, HLA-DQ8 mice treated with these two SAgs showed an acute tumoricidal effect and survived a lethal challenge of unrelated Lewis lung carcinoma or B16F10 melanoma for more than 360 days while showing no significant toxicity. Unexpectedly, we further discovered that, despite SEI's ability to induce the highest levels of TNFα in human T cells of all SAgs (Terman et al. supra 2013), SEI used together with SEG in HLA-DQ8 mice displayed very low levels of TNFα while simultaneously producing massive quantities of IFNγ. IFNγ has the ability to exert direct anti-tumor effects on tumor cells or indirect tumoricidal activity via activation of immune cells (Ikeda H et al. Cytokine and Growth Factor Rev. 13 95-109 (2002); Tan J et al. J Immunother. 21:48-55. (1998); Parker B S et al. Nature Rev. Cancer 16: 131-140 (2016)). Ratios of serum levels of IFNγ:TNFα observed during SEG-SEI treatment ranged from 3000:1 to 800:1 far exceeding those noted in the course of SEB and SPEA immunization in humanized transgenic mice (Bavari S et al. J. Infect. Dis. 186:501-10 (2002); Norrby-Teglund A et al. Eur. J Immunol. 32: 2570-2577 (2002); Llewelyn M et al. J Immunol. 172:1719-1726 (2004); Tilahun A Y et al. Mediators of Inflammation doi.org/10.1155/2014/468285; Rajagopalan G et al. Tissue Antigens 71:135-145 (2007)). These elevated IFNγ levels promoted the tumor killing effect of SEG and SEI while low TNF levels precluded toxicity. Hence, in contrast to canonical SAgs, the unique partnership of SEG-SEI with endogenous HLA-DQ8 in MHCII transgenic mice produced a potent acute and long-term tumoricidal response entirely devoid of toxicity.

SAg-induced T effector cell and cytokine responses are critically reliant on their functional interaction with endogenous MHCII. Thus, in addition to T cell activation most SAgs produce high levels of toxicity-inducing cytokines such as TNFa. This explains the high prevalence of toxic shock induced by these agents in humans and in MHCII humanized mice. Indeed, the nature of the SAg-MHCII pairing in humans has been shown to be a critical determinant of the severity of toxicity in response to streptococcal SAg in streptococcal pyogenes-induced sepsis (Kotb M et al. Nat Med. 8, 1398-13404 (2002)); Norrby-Teglund A et al., Eur J Immunol. 32, 2570-2577 (2002)). We therefore commenced a search for human MHC class II allotypes that could best condition SEG-SEI for a tumoricidal response in vivo devoid of the disabling effects of toxic cytokines. To this end, turned to transgenic models which express major histocompatibility complex (MHC) class II human leukocyte antigen (HLA)-DR3 (DRB1*0301) or DQ8 alleles in the absence of murine MHCII expression on a large percentage of myeloid cells (Cheng S et al. Eur J Immunogenet. 23,15-20 (1996); Maiersa M et al. Human Immunology 68, 779-788 (2007); Mangalam A et al., J Immunol. 182, 5131-1539 (2009)). These models are representative of the two major human MHCII allotypes and have been widely used for studies of human autoimmunity and infectious disease (Mangalam A K et al. Adv Immunol. 97, 65-147 (2008); DaSilva et al. J Infect Dis. 185,1754-60 (2002)). Because these allotypes are more efficient at SAg presentation than murine MHCII, humanized MHCII transgenic mice are preferred for functional assessment of T cell responses and toxicity elicited by SAgs (Welcher B C, J Infect. Dis. 186, 501-510 (2002); Rajagopalan G, Int Immunol. 14, 801-12 (2002)). To date, however, these models have not been deployed to assess SAg- and/or tumor neopeptide driven anti-tumor effects.

The human HLA-DQ8 allele (DQA*03:01, DQB*03:02) was of particular interest. It comprises two highly polymorphic α-chain and β-chain variants that engage SAgs and a broad repertoire of endogenous and exogenous self and microbial peptides via a large antigen binding groove with less stringent length and sequence requirements than other MHCII alleles (vanLummel M et al. J Biol Chem 287, 9514-9524 (2012); Busch R et al. Expert Rev Mol Med. July 6; 14:e15. doi: 10.1017/erm.2012.9.; Zhou Z et al. Front Immunol. 4:262-271 (2013)). Notably, human tumor neopeptides, positively selected during tumorigenesis also reportedly bind HLA-DQ in preference to HLA-DR alleles. High throughput epitope discovery has further shown frequent recognition of human melanoma neoantigens by CD4+ T cells (Marty Pyke R Cell. 175, 416-428 (2018); Linnemann C et al. Nat Med 21, 81-85 (2015); Ott P A, Nature 547, 217-221 (2017)). Likewise, in mouse solid tumor models including the B16 melanoma, a larger portion of the immunogenic tumor mutanome was presented by MHC-II than MHC-I and recognized by CD4 T cells (Kreiter S, et al. Nature 520, 692-696 (2015). Despite these properties, to our knowledge, the HLA-DQ8 allele has heretofore not been recognized or exploited for tumor therapy as a distinct component of an anti-tumor therapeutic complex. Here it is repurposed in a therapeutic role as a key constituent of a complex with SAgs.

The instant invention remedies problems posed by the prior art by selecting SEG-SEI and showing that its contact with the endogenous HLA-DQ8 allele humanized mice and exposure to the immune system produced an acute and long-term tumoricidal response entirely devoid of toxicity. SEG-SEI-induced a CD4+- and CD8+ T-cell dependent anti-tumor response marked by chemotactic recruitment of the CD8+ effector T-cells to the TME, appearance of tumoricidal cytokines IFNγ/TNFα and subdued levels of Tregs and myeloid cells. Most importantly, we found that SEG-SEI in partnership with endogenous HLA-DQ8 produced high levels of T effector cells and IFNγ together with low levels of TNFa. Long-term survivors exhibited specific anti-melanoma memory, rejecting melanoma but not the unrelated LLC. Histopathology corroborated the major tumoricidal effect and demonstrated treatment-induced conversion of the melanoma from “cold” to “hot” (i.e. infiltrated by T effector T cells).

This anti-tumor response without toxicity was remarkable since several canonical SAgs such as SEB, SEC and SpeA tested in the same humanized model produced toxic shock. The HLA-DQ8 was obligatory since identical SEG-SEI treatment in tumor bearing HLA-DR3 mice produced toxic shock and no anti-tumor response. Likewise, SEG-SEI were requisite since canonical SAgs induced undue toxicity in the very same HLA-DQ8 mice. Importantly, tumor specificity of the long term response suggested that tumor neoantigens were pivotal in directing early priming of tumor reactive T cell population that was expanded and differentiated into potent T effector cells by the addition of the SEG-SEI-(HLA-DQ8) complex.

Further objects and advantages of this invention will become apparent from a consideration of the drawings and ensuing description.

### SUMMARY OF INVENTION

Superantigens (SAgs) are the most potent T-cell agonists whose strength and quality are governed by their binding topology with MHCII, TCR and B7/CD28 axis. Amid their enormous biologic diversity, we have discovered a new group of evolutionarily modified SAgs, SEG-SEI, that in partnership with endogenous SEG-SEI retain the ability to generate anti-tumor T effector cell devoid of the cytokine-mediated toxicity. Such toxicity has hampered the effective use of canonical SAgs for human cancer treatment. For their MHCII partner, we selected the human HLA-DQ8 allele and show that its contact with SEG-SEI is obligatory for the anti-tumor effect. Here we further show that SEG-SEI collaborate with HLA-DQ8 alleles to expand, differentiate and chemotactically recruit a tumor neoantigen-primed tumor reactive T cell population that propagates both an acute tumoricidal response and long-term T-cell memory/survival.

### LEGENDS TO FIGURES

FIG. 1A, FIG. 1B, FIG. 1C, FIG. 1D, FIG. 1E, FIG. 1F, FIG. 1G. Evaluation of superantigenic strength, T cell effector and Treg differentiation in response to SEG, SEI and SEG-SEI in vitro. CD4+ and CD8+ splenocytes from HLA-DQ8 tg, HLA-DR3 tg and C57BL/6 were incubated with SEG, SEI, SEG-SEI, SEB and SEA (1 pg/ml) for 72 hours and assayed for proliferation (FIG. 1A, FIG. 1B). CD4+ and CD8+ T cells from HLA-DQ8 tg, HLA-DR3 tg and C57BL/6 were incubated with SEG, SEI or SEG-SEI (1 μg/ml) for 72 hours and assayed for IFNγ (FIGS. 1C,1D) or granzyme and perforin (FIGS. 1E,1F). CD4+ T cells from HLA-DQ8 tg, HLA-DR3 tg and C57BL/6 mice were incubated with SEG, SEI or SEG-SEI (1 μg/ml) for 72 hours and FoxP3 expression was quantified (FIG. 1G). Data expressed as mean SD. ANOVA with Tukey posttest, n=3-5. *p<0.05, **p<0.01, ***p<0.001, ****p<0.001.

FIG. 2A, FIG. 2B, FIG. 2C, FIG. 2D, FIG. 2E, FIG. 2F, FIG. 2G. Survival of mice bearing established B16-F10 melanoma treated with SEG-SEI is shown. (FIG. 2A) C57BL/6 or (FIG. 2 B) HLA-DQ8 tg mice received 2.5×10⁵ B16-F10 cells ip on day 0. SEG-SEI 50 μg of each was administered on days 6 and 9. Controls received B16-F10 tumor cells but no treatment (Kaplan Meier with Log-rank n=10⁻¹¹). (FIG. 2C) On day 160, 4 surviving mice from (FIG. 2B) were rechallenged with 2.5×10⁵ B16-F10 cells ip. Untreated controls received B16-F10 tumor alone. (FIG. 2D) An additional 5 mice from (FIG. 2 that survived 160 days were challenged with 2.5×10⁵ Lewis lung carcinoma cells (LLC). Untreated controls received LLC cells alone (Kaplan Meier with Log-rank). Histopathology analysis of B16-F10 omental tumors on day 13 after tumor inoculation from (FIG. 2E) untreated and (FIG. 2F) SEG-SEI treated HLA-DQ8 tg mice is shown (H&E ×10 magnification). (G) HLA-DQ8 tg mice received 2.5×10⁵ live B16-F10 cells ip on day 0, followed by the treatment with SEG-SEI 50 μg of each on days 6 and 9 and ip injections of anti-CD4, anti-CD8 and anti-IgG2b isotype control as shown (red arrows) (Kaplan Meier with Log-rank, n=4-5) *p<0.05, **p<0.01.

FIG. 3A, FIG. 3B, FIG. 3C, FIG. 3D. Evaluation of T cells and myeloid cells in B16-F10 tumors from HLA-DQ8 tg mice treated with SEG-SEI is shown. HLA-DQ8 tg mice received 2.5×10⁵ live B16-F10 cells ip on day 0, followed by the treatment with SEG-SEI 50 μg of each on days 6 and 9. Controls were inoculated with tumor but received no treatment. On day 13, tumors were excised and assayed for (FIG. 3A) CD3+CD8+ cells, (FIG. 3B) CD8‘granzyme B’ cells, (FIG. 3C) CD25⁺FoxP3⁺ cells, (FIG. 3D) CD11b⁺GR1⁺ cells. ANOVA with Tukey posttest. n=3. *p<0.05, **p<0.01, ***p<0.001, ****p<0.001. Ratio of CD8⁺ granzyme B⁺ cells to CD25⁺Foxp3⁺ cells=>10:1 (n=4, p=0.01) and ratio CD8+ granzyme B+ cells to CD11b+GR1+ cells=3.9:1 (n=4, p=0.03). Data expressed as mean±SD. Student's T test, two-tailed.

FIG. 4A, FIG. 4B, FIG. 4C, FIG. 4.D, FIG. 4E,F, FIG. 4G, FIG. 4H, FIG. 4I, FIG. 4J. Evaluation of cytokines and chemokines in serum and tumor from HLA-DQ8 tg mice treated with SEG-SEI is shown. (FIG. 4A, FIG. 4B, FIG. 4C, FIG. 4.D, FIG. 4E,F, FIG. 4G, FIG. 4H) HLA-DQ8 tg and HLA DR3 tg mice received 2.5×10⁵ B16-F10 cells ip and were treated with SEG-SEI, 50 μg of each ip on days 6 and 9. Controls received tumor inoculation but no SEG-SEI treatment. Serum cytokines were analyzed 24 hours after each SEG-SEI treatment. n=4-5. (FIG. I, FIG. J) HLA-DQ8 tg mice received 2.5×10⁵ live B16-F10 cells ip on day 0, followed by the treatment with SEG-SEI, 50 μg of each, on days 6 and 9. Controls were inoculated with B16-F10 cells ip but received no treatment. On days 13 and 16 tumors were assayed for (FIG. I) cytokines and (FIG. J) chemokines respectively. Data expressed as mean±SD. ANOVA with Tukey posttest. n=3. *p<0.05, **p<0.01, ***p<0.001, ****p<0.0001.

FIG. 5A, FIG. 5B, FIG. 5C, FIG. 5D, FIG. 5E, FIG. 5F, FIG. 5G FIG. 5H, FIG. 5I, FIG. 5J, FIG. 5K. T cell recruitment to tumor injection site after administration of SEG-SEI to HLA-DQ8 tg mice is shown. HLA-DQ8 tg mice received 2.5×10⁵ B16-F10 cells ip and were treated with SEG-SEI 50 μg of each ip on days 6 and 9. Controls were inoculated with tumor but received no treatment. Twenty-four hours after each SEG-SEI treatment peritoneal lavage and peripheral blood were assayed for (FIG. 5A, FIG. 5B) CD4+ and CD8+ T cells, (FIG. 5C, FIG. 5D) perforin-granzyme B+T effector cells, and (FIG. 5E, FIG. 5F) CD44+CD62L− cells. (FIG. 5G, FIG. 5H, FIG. 5I, FIG. 5J) Omental tumor sections on day 10 in untreated mice and SEG-SEI treated mice were analyzed for histopathology. H&E ×10 magnification (FIG. 5G, FIG. 5I); ×40 magnification (FIG. 5I, FIG. 5J). Quantification of histologic necrosis in untreated and SEG-SEI treated tumor sections on days 7 and 10 is shown is shown (FIG. 5K). n=3-4. Data expressed as mean SD. *p<0.05, **p<0.01, ***p<0.001, ****p<0.001

FIG. 6A, FIG. 6B. Vaccination with inactivated B16-F10 melanoma plus SEG-SEI in HLA-DQ8 tg mice induces long term survival. (FIG. 6A) HLA-DQ8 tg mice were treated with 1×10⁶ irradiated (15,000 rads) B16-F10 melanoma cells ip, day −13 and injected with SEG-SEI 50 μg of each ip on days −7 and −3. Mice were challenged with 2.5×10⁵ live B16-F10 cells ip day 0. Controls received live melanoma challenge alone, irradiated B16-F10 cells plus live melanoma challenge or SEG-SEI plus live melanoma cells. HLA-DQ8 tg mice surviving >60 days (“X”) were rechallenged with 2.5×10⁵ live B16-F10 cells ip. (FIG. 6B)HLA-DQ8 tg mice were inoculated with 1×10⁶ irradiated (15,000 rads) B16-F10 melanoma cells on day −13 and injected with SEG-SEI 50 μg of each ip on days −7 and −3. Mice were challenged with 2.5×10⁵ live B16-F10 cells or Lewis lung carcinoma cells on day 0. Controls received 2.5×10⁵ B16-F10 cells or Lewis lung carcinoma cells alone. (R)LLC+SEG-SEI+(C) LLC vs LLC, p<0.05; (R)B16+SEG-SEI+(C)B16 vs LLC, p<0.001 where (R) indicates irradiated tumor cells and (C) indicates live tumor challenge. Kaplan Meier curves with Log-rank test, n=10. *p<0.05, **p<0.01, ***p<0.001.

FIG. 7A, FIG. 7B, FIG. 7C, FIG. 7D, FIG. 7E, FIG. 7F, FIG. 7G, FIG. 7H, FIG. 7I, FIG. 7 J. Recall response of vaccinated HLA-DQ8 mice to melanoma challenge is shown. HLA-DQ8 tg mice were vaccinated with 1×10⁶ irradiated B16-F10 cells on day −13, treated with SEG and SEI, 50 μg of each, on days −7 and −3 and challenged with 2.5×10⁵ live B16-F10 cells on day 0. On day 40, they were rechallenged 2.5×10⁵ live B16-F10 cells and 6 days later (day 46) omental peritoneal lavage were examined. Unvaccinated HLA-DQ8 tg mice inoculated with tumor cells and sacrificed 6 days later served as controls. Analysis of peritoneal lavage showed increased levels of CD127+ and CD44+CD62L− CD4+ and CD8+ T cells (FIG. 7A, FIG. 7B, FIG. 7C, FIG. 7D). Sections of omental tumor from untreated (FIG. 7E, FIG. 7F) and vaccinated (FIG. 7G, FIG. 7H ) are shown. H&E, magnification ×5 (FIG. 7E, FIG. 7,G) and ×40 (FIG. 7F, FIG. 7I). Quantitation of tumor density in sections of treated and vaccinated mice is shown using the ImageJ software (FIG. 7I). HLA-DR3 tg mice received 2.5×10⁵ B16-F10 cells ip and were treated with SEG and SEI 50 μg of each ip on days 6 and 9. Controls received tumor inoculation but no treatment (FIG. 7J). Data expressed as mean SD; Kaplan Meier curves with Log-rank test, n=10. *p<0.05, **p<0.01, ***p<0.001.

FIG. 8. Chain of events in SEG-SEI-treated HLA-DQ8 tg mice leading to long-term anti-tumor memory and melanoma survival is shown. Following melanoma inoculation on day 0, HLA-DQ8 expressing APCs recognize melanoma neoantigens and present them to T cells resulting in priming of tumor reactive CD4+ and CD8+ T cells. This priming alone is insufficient to induce an anti-tumor response. Introduction of SEG-SEI on days 6 and 9 results in expansions of both CD4+ and CD8+ T-cells. The CD4+ T-cell population may enhance the early melanoma antigen priming process and promote CD8+ T cell differentiation into T effector cells. The superantigenic T cell activation also generates T cell chemotactic molecules that enables recruitment of T effector cells to the tumor. SEG-SEI further initiates a surge of IFNγ from both CD4+ and CD8+ T-cells. By days 13-16, the TME is infiltrated by CD8+T effector cells along with IFNγ and TNFα that appear to constitute the major tumoricidal effectors. CD8+T effector cell tumor killing may be enhanced by the relative paucity of Tregs and myeloid cells in the TME. Histopathology at this time corroborated a major anti-tumor effect. Long-term surviving SEG-SEI-treated mice exhibit specific anti-tumor memory by resisting late challenge with melanoma cells while succumbing to inoculation with unrelated Lewis lung carcinoma. The long-term anti-tumor response suggests a self-sustaining feed forward mechanism possibly mediated by SEI persistence in vivo in a high affinity, Zn⁺⁺ dependent interaction with the HLA-DQ8 allele. The key role of the HLA-DQ8 allele in unleashing SEG-SEI's anti-tumor effect was underscored by the absent anti-tumor response in identically treated HLA-DR3 tg mice.

FIG. 9A FIG. 9B. (FIG. 9A) Evaluation biocompatibility of HLA-DQ8 tg and C57BL/6 cells in mixed lymphocyte cultures is shown. Mitomycin C-treated C57BL/6 splenocytes (red) were cocultured with splenocytes from HLA-DQ8 tg mice for 3 days at 37° C. in 5% CO₂. CD3+ T cell proliferation (green) was measured by dye dilution via flow cytometry. (FIG. 9B) The ability of HLA-DQ8 tg mice to reject the unrelated 4T1 mammary carcinoma indigenous to Balb/C mice was evaluated. HLA-DQ8 tg or BalbC mice received 2.5×10⁵ 4T1 tumor cells ip on day 0 and survival was assessed. Kaplan Meier with Log-rank, n=6. ***p<0.001.

FIG. 10A, FIG. 10B. B16-F10 melanoma is established at day 6 after tumor inoculation in the omentum of both HLA-DQ8 tg and C57BL/6 mice. B16-F10 cells 2.5×10⁵ live were injected ip into 6-8-week-old female HLA-DQ8 tg and C57BL/6 mice. (FIG. 10A) Images of the peritoneal cavity (left) and omental tissue (right) show the presence of established melanoma tumor masses of comparable size in both strains (dotted white line). (FIG. 10B) Histopathologic sections of omental tumor from both strains on day 6 after tumor implant show tightly packed melanoma cells with established stroma and vasculature. (H & E ×25 magnification).

FIG. 11. The ability of YTB to prevent the outgrowth of B16F10 melanoma in HLA-DQ8 mice was examined in the intraperitoneal tumor model. B16F10 cells (2.5×10⁵) were injected intraperitoneally in groups of 5 mice. The experimental group received live YTB in doses of 10⁴ ip on days −2 and +4. Live B16F10 cells were delivered ip on day 0. Control group was injected ip with 10⁵ B16F10 tumor cells alone but received no treatment. n=5, Log-rank (Mantel-Cox) p<0.01.

### DESCRIPTION OF PREFERRED EMBODIMENTS

### SEG-SEI

Staphylococcus aureus produces a broad range of exoproteins, including staphylococcal enterotoxins and staphylococcal-like enterotoxins. All these toxins exhibit superantigenic properties activating a large proportion of T cells after binding to the major histocompatibility complex (MHC) class II molecule and crosslinking specific v regions of the T-cell receptor (TCR). This interaction results in polyclonal T-cell activation and secretion of cytokines such as interferon gamma (IFNγ), tumor necrosis factor alpha (TNFa), and nitric oxide (NO). Several members of this group have a major role in the pathogenesis of toxic shock syndrome and food poisoning and exhibit anti-tumor activity in animal models. Unprocessed SAgs bind directly to MHCII molecules outside the polymorphic antigen-binding groove used by conventional peptides and are capable of activating T lymphocytes at picomolar concentrations. Superantigen binding to selective vβ chains of the T cell receptor (TCR) activates up to 20% of resting T cells relative to conventional antigens which stimulate less than 1% of the T cell repertoire. (Marrack, P., and Kappler J Science 248: 1066-72 (1990)); Terman, D. S. et al., Clin. Chest Med. 27: 321-34 (2006)).

The egcSEs comprise five genetically linked staphylococcal enterotoxins, SEG, SEI, SEM, SEN and SEO and two pseudotoxins which constitute an operon present in up to 80% of S. aureus isolates. The egcSEs are structurally homologous and phylogenetically related to classic SEA-E (Jarraud, S. et al. J. Immunol. 166: 669-77 (2001); Becker, K. et al. J. Clin. Microbiol. 41:1434-9 (2003))). Despite their broad distribution, human serum levels of neutralizing antibodies directed against the egcSEs are significantly lower than those directed to the classic SEs (Holtfreter et al. supra (2004)). This has been largely ascribed to weak transcription and suboptimal toxin secretion by the parental Staphylococcus aureus (Roetzer A et al. supra (2016). Interestingly, septicemia associated with the egcSEs appears to be less severe clinically than that linked to the classic SEs (Ferry et al. Clin. Microbiol. Infect. 14: 546-554 (2008)). In recent studies, the egc SEs showed a hierarchy of T cell activation and the ability to generate cytotoxic nitrites and TH-1/TH-2 cytokines (Terman et al. supra (2013)).

SEG resides within the ege operon of Staphylococcus aureus. It occurs usually with other SEs in up to 80% of S. aureus isolates (Munson S et al. Infect. Immun. 66: 3337-3348 (1998); Banks et al. J. Infect. Dis. 187: 77-86 (2003); Becker et al. Clin. Microbiol. 41: 1434-1439 (2003)). SEG consists of 777 nucleotides encoding a mature protein of 233 residues, 27,043 Da. which shares 41 to 46% amino acid sequence identity with other members of the SEB family (SSA 46%, SEB 45%, SPEA 43%, SEC2-3 42%, SEC1 41%).

Notably, SEG shows substitutions in three key residues located in the conserved binding surface for murine V8.2, resulting in an affinity for VP8.2 (KD 0.125 uM by SPR) which is 40 to 100-fold higher than that reported for other members of SEB subfamily and the highest reported for any wild type SAg-TCR couple. SEG also retains the fast dissociation rate characteristic of SAgs that allows sequential binding to several other TCR vβ molecules (Fernandez M M J. Biol. Chem. 286: 1189-1195 (2011)). Studies of recombinant SEG demonstrated superantigenic vβ-dependent expansion of vβ3,12,13A,13B,14,15 T cells (Seo K S, et al. J Transl Med 8: 1-9 (2010)). SEG produced T cell proliferation with an ED₅₀ of 15 fM compared to SEA=<1 fM and SEG homologue SEG-R47 (wtSEG with _{lys}47_{arg} substitution)=R47=4 nM all well within superantigenic range. SEG also induced nitrous oxide and TNFα dependent killing of abroad range of tumor cells including human lung, head and neck, colon and breast carcinoma cell lines (Terman et al. supra (2013). Inoculation of SEG into the right hind footpad of AKR/J mice showed the proliferation of mVbP8.2 T-cells in regional lymph nodes was twice as great as in mice inoculated with SEC3; and the stimulation effect extended to lymph nodes from the left hind leg inoculated with PBS. Despite its high affinity for MHCII SEG generates lower levels of TH-1 cytokines, IFNγ, TNF-α, IL-2 than the other egcSEs and classical SEA. Unlike the canonical SAgs, SEG exhibits a low incidence of sero-reactive naturally occurring antibodies (Holtfreter S et al. supra (2004). Such antibodies nullified the therapeutic effect of SAgs, contributed to their toxicity, and narrowed the number of human cancer patients eligible for treatment (Alpaugh R et al. supra (1998). SEG also induces lower levels of TH-1 cytokines relative to classic SEA e.g., TNFα (p=0.004); IFN-γ (p<0.001); IL-2 (p<0.001); IL-17 (p<0.001); GM-CSF (p=0.07) (Terman et al, supra (2013); Dauwalder et al. J. Leuk Biol. 80:1-6 (2006)).

SEI is encoded in the egc operon of S. aureus and associated with toxic shock syndrome, food poisoning. With respect to SEI, crystallographic studies have shown that it belongs to the zinc family bind to a high affinity site on the MHCII β-chain. SEI targets conservatively substituted residues of the polymorphic β-chain. (Fernandez M M et al. J. Biol Chem 281: 25356-25364 (2006)). SEI induces robust T cell activation selectively stimulating vβ1,5,6,23. It also exhibits the highest levels of IFNγ, TNFα and IL-2 of all egcSEs (Terman et al. supra (2013)).

Amino acid sequences for SEG and SEI are given below:

SEQ ID NO: 7
SEG (Baba,T. et al., Lancet 359, 1819-1827 (2002))

| | |
|---|---|
| 1 | MNKIFRVLTV SLEFFTELIK NNLAYADVGV INLRNEYANY |
| | QPEKLQGVSS GNFSTSHQLE |
| 61 | YIDGKYTLYS QFHNEYEAKR LKDHKVDIFG ISYSGLCNTK |
| | YMYGGITLAN QNLDKPRNIP |
| 121 | INLWVNGKQN TISTDKVSTQ KKEVTAQEID IKLRKYLQNE |
| | YNIYGENKTK KGQEYGYKSK |
| 181 | FNSGFNKGKI TFHLNNEPSF TYDLFYTGTG QAESFLKIYN |
| | DNKTIDAENF HLDVEISYEK |
| 241 | TE |

SEQ ID NO: 8
SEI (Kuroda, M. et al., Lancet 357 (9264), 1225-
1240 (2001))

| | |
|---|---|
| 1 | MKKFKYSFIL VFILLFNIKD LTYAQGDIGV GNLRNFYTKH |
| | DYIDLKGVTD KNLPIANQLE |
| 61 | FSTGTNDLIS ESNNWDEISK FKGKKLDIFG IDYNGPCKSK |
| | YMYGGATLSG QYLNSARKIP |
| 121 | INLWVNGKHK TISTDKIATN KKLVTAQEID VKLRRYLQEE |
| | YNIYGHNNTG KGKEYGYKSK |
| 181 | FYSGFNNGKV LFHLNNEKSF SYDLFYTGDG LPVSFLKIYE |
| | DNKIIESEKF HLDVEISYVD |

Over time, these SEs have undergone evolutionary structural changes that have modulated the quality strength of their superantigenic T cell stimulation and cytokine output and distinguished them from canonical SEs. In a functional assessment, both SEG and SEI induced potent T cell activation while the SEG produced the lowest levels of TNFα and SEI the highest levels of IFNγ (Terman D S et al. supra (2013). These biologic properties coupled with minimal neutralizing antibodies in human sera suggested that these agents might work together to produce a more favorable therapeutic index against cancer than previously deployed canonical SEs.

### HLA-DQ8

MHC class II molecules in mice and humans are the primary docking sites for bacterial superantigens. In humans, there are three major isotypes of HLA class II, designated HLA-DP, HLA-DQ and HLA-DR. HLA-DR and HLA-DQ exist in multiple allelic forms. HLA-DQ class II molecule is a heterodimer consisting of an alpha (DQA) and a beta chain (DQB), both anchored in the membrane. It plays a central role in the immune system by presenting peptides derived from extracellular proteins. Class II molecules are expressed predominantly on antigen presenting cells such as B Lymphocytes, dendritic cells, macrophages. The alpha chain is approximately 33-35 kDa. It is encoded by 5 exons; exon 1 encodes the leader peptide, exons 2 and 3 encode the two extracellular domains, and exon 4 encodes the transmembrane domain and the cytoplasmic tail. Within the DQ molecule both the alpha chain and the beta chain contain the polymorphisms specifying superantigen and peptide binding specificities.

The affinity and conformation SAgs for HLA class II alleles are critical determinants of the strength and quality of the T cell response. Indeed, HLA class II polymorphism thereby controls the diversity of T cell proliferative and cytokine responses to any given SAg (Bell J I et al. Immunol. Rev. 84: 51-71 (1985); Turner D. Vox. Sang. 87 Suppl 187-90 (2004); Ovsyannikova I G et al. J Infect. Dis. 193: 655-63. (2006); Monos D S et al. Human Immunol. 66: 554-62 (2005)). In a typical example, superantigen streptococcal pyrogenic exotoxin A induced higher proliferative responses and production of IFNγ, TNFα and IL-2 when presented by MHCII DQA1*010/DB1*03 as compared to MHCDR1, DR4 and DR5 (Norrby-Teglund et al., Eur. J. Immunol. 32: 2570-2577 (2002)). The optimal T cell profile for SAg would be a high degree of CD4+, CD8+ T cell expansion, T effector cell generation and IFNγ production to preserve the tumor killing effect along with muted levels of toxicity producing TNFα. To date there is no identifiable SAg-MHCII combination that displays T cell activation and IFNγ surge with minimal TNFα that could be used for anti-tumor treatment in humans without significant toxicity.

Given the critical reliance of SAg functionality on the underlying MHCII platform we commenced a search for human MHC class II allotypes that could best condition SEG-SEI for a tumoricidal response in vivo without toxicity. To this end, we turned to transgenic models which express major histocompatibility complex (MHC) class II human leukocyte antigen (HLA)-DR3 (DRB1*0301) or DQ8 alleles in the absence of murine MHCII expression on a large percentage of myeloid cells (Mangalam A et al. J Immunol. 182, 5131-1539 (2009). HLA-DR or DQ were expressed on 35-50% of cell populations in PBLs and splenocytes and a normal T cell repertoire of these respective models. They also display a standard T cell repertoire with a normal number of CD4 and CD8 cells in spleen, PBLs, and LNCs and their T cells shown CD4 and CD8 T cells also showed a diverse T cell Vβ repertoire. These models are representative of the two major human MHCII allotypes and have been widely used for studies of human autoimmunity and infectious disease (Welcher et al. supra (2002); Taneja V and David C S. Immunol. Rev. 169: 67-79 (1999)). Because these allotypes are more efficient at SAg presentation than murine MHCII, humanized MHCII transgenic mice are preferred for functional assessment of T cell responses and toxicity elicited by SAgs (Welcher B C et al. J. Infect. Dis. 186, 501-510 (2002). Rajagopalan G et al. Int Immunol. 14, 801-12 (2002)). Indeed, many SAgs that were tested in the MHCII transgenic models showed significant toxicity whereas they were non-toxic in wild type murine models. To date, however, these models have not been deployed to assess SAg and/or tumor neopeptide driven anti-tumor effects.

The human HLA-DQ8 allele (DQA*03:01, DQB*03:02) was of particular interest. It comprises two highly polymorphic α-chain and β-chain variants that engage SAgs and a broad repertoire of endogenous and exogenous self and microbial peptides via a large antigen binding groove with less stringent length and sequence requirements than other MHCII alleles (44-46). Notably, human tumor neopeptides, positively selected during tumorigenesis also reportedly bind HLA-DQ in preference to HLA-DR alleles. High throughput epitope discovery has further shown frequent recognition of human melanoma neoantigens by CD4+ T cells (Marty Pyke R Cell. 175, 416-428 (2018). Linnemann C et al. Nat Med. 21, 81-85 (2015); Ott P A Nature 547, 217-221 (2017)). Likewise, in mouse solid tumor models including the B16 melanoma, a larger portion of the immunogenic tumor mutanome was presented by MHC-II than MHC-I and recognized by CD4 T cells (Kreiter S, et al. Nature 520, 692-696 (2015). Despite these properties, to our knowledge, the HLA-DQ8 allele has heretofore not been exploited for tumor therapy with either SAgs or neopeptides. HLA-DQ8-tg) mice provided an opportunity to assess SEG-SEI in an animal model with a sensitivity to SAgs similar to that of humans.

The nucleic acid sequences for HLA-DQ8 alpha and beta chains are given below.

His-tag
SEQ ID NO: 1
ATGCATCACcatCACCATCC
DQ8A1 cDNA
SEQID NO: 2

| | atcc |
|---|---|
| 61 | taaacaaagc tctgctgctgggggccctcgctctgaccac |
| | cgtgatgagc ccctgtggag |
| 121 | gtgaagacat tgtggctgac cacgttgcct cttgtggtgt |
| | aaacttgtac cagttttacg |
| 181 | gtccctctgg ccagtacacc catgaatttg atggagatga |
| | gcagttctac gtggacctgg |
| 241 | agaggaagga gactgcctgg cggtggcctgagttcagcaa |
| | atttggaggttttgacccgc |
| 301 | agggtgcactgagaaacatg gctgtggcaa aacacaactt |
| | gaacatcatg attaaacgct |
| 361 | acaactctac cgctgctacc aatgaggttc ctgaggtcac |
| | agtgttttcc aagtctcccg |
| 421 | tgacactggg tcagcccaac accctcattt gtcttgtgga |
| | caacatcttt cctcctgtgg |
| 481 | tcaacatcac atggctgagc aatgggcagtcagtcacaga |
| | aggtgtttctgagaccagct |
| 541 | tcctctccaa gagtgatcat tccttcttca agatcagtta |
| | cctcaccttc ctcccttctg |
| 601 | ctgatgagat ttatgactgc aaggtggagc actggggcct |
| | ggaccagcct cttctgaaac |
| 661 | actgggagcc tgagattcca gcccctatgt cagagctcac |
| | agagactgtg gtctgtgccc |
| 721 | tggggttgtc tgtgggcctc atgggcattg tggtgggcac |
| | tgtcttcatc atccaaggcc |
| 781 | tgcgttcagttggtgcttcc agacaccaag ggccattgtga |

C-myctag
SEQ ID NO: 3
gcc acc ATG GAA CAA AAA CTT ATT TCT GAA GAA
DQB1 cDNA
SEQ ID NO: 4

| | |
|---|---|
| | tct |
| 61 | tggaagaagg ctttgcggat ccctggaggc cttcgggtag |
| | caactgtgac cttgatgctg |
| 121 | gcgatgctga gcaccccggt ggctgagggc agagactctc |
| | ccgaggattt cgtgtaccag |
| 181 | tttaagggca tgtgctactt caccaacggg acggagcgcg |
| | tgcgtcttgt gaccagatac |
| 241 | atctataacc gagaggagta cgcacgcttc gacagcgacg |
| | tgggggtgta tcgggcggtg |
| 301 | acgccgctgg ggccgcctgc cgccgagtac tggaacagcc |
| | agaaggaagt cctggagagg |
| 361 | acccgggcgg agttggacac ggtgtgcaga cacaactacc |
| | agttggagct ccgcacgacc |
| 421 | ttgcagcggc gagtggagcc cacagtgacc atctccccat |
| | ccaggacaga ggccctcaac |
| 481 | caccacaacc tgctggtctg ctcagtgaca gatttctatc |
| | cagcccagat caaagtccgg |
| 541 | tggtttcgga atgaccagga ggagacaact ggcgttgtgt |
| | ccacccccct tattaggaac |
| 601 | ggtgactgga ccttccagat cctggtgatg ctggaaatga |
| | ctccccagcg tggagacgtc |
| 661 | tacacctgcc acgtggagca ccccagcctc cagaacccca |
| | tcaccgtgga gtggcgggct |
| 721 | cagtctgaat ctgcccagag caagatgctg agtggcattg |
| | gaggcttcgt gctggggctg |
| 781 | atcttcctcg ggctgggcct tattatccat cacaggagtc |
| | agaaagggct cctgcac |

P2A-Sequence
SEQ ID NO: 5
GGAAGCGGAGCTACTAACTTCAGCCTGCTGAAGCAGGCTGGAG
ACGTGGAGGAGAACCCTGGACCT
Amino acid sequence of translated polycistronic
DQ8 alpha and beta chains
(SEQ ID NO: 6)
ATMEQKLISEESWKKALRIPGGLRVATVTLMLAMLSTPVAEGRDSPEDFV
YQFKGMCYFTNGTERVRLVTRYIYNREEYARFDSDVGVYRAVTPLGPPAA
EYWNSQKEVLERTRAELDTVCRHNYQLELRTTLQRRVEPTVTISPSRTEA
LNHHNLLVCSVTDFYPAQIKVRWFRNDQEETTGVVSTPLIRNGDQTFQIL
VMLEMTPQRGDVYTCHVEHPSLQNPITVEWRAQSESAQSKMLSGIGGFVL
GLIFLGLGIIHHSQKGLLHGSGATNFSLLKQAGVEENPGPMHHHHHHILL
NKALLLGALALTTVMSPCGEDIVADHASCGVNLYQFYFPSFQFYTHEFDF
DEQFHVDLERKETAWRWPEFSKFGGDPQFALRNMAVAKHLNIMIKRYNST
AATNEVPEVTVFSKSPVTLGQPNTLICLVDNIFPPVVNITWLSNGQSVTE
GVSETSGSKSDHSFFKISYLTFLPSADEIYDCKVEHWGLDQPLLKHWEPE
IPAPMSELTETVVCALGLSVGLMGIVVGTVFIIQGLRSVGASRHQGPL

By using SEG and SEI together in humanized HLA-DQ8 mice we have found that these agents show striking ant-tumor effects devoid of toxicity including weight loss, cachexia or death. Notably, the anti-tumor response was dependent on HLA-DQ8 expression since SEG-SEI treatment of HLA-DR3 mice showed no anti-tumor effect. Likewise, SEG-SEI were requisite since other SAg used in HLA-DQ8 mice showed undue toxicity. SEG-SEI induced a strong T effector cell and IFNγ response and was capable of chemotactically recruiting T effector cells to the tumor microenvironment. The combination produced minimal toxicity due muted TNFa production. The effectiveness of T effector/memory cells in tumor killing was likely enhanced by the minimal presence of Tregs and myeloid cells in the TME of SEG-SEI-treated HLA-DQ8 tg mice (Srinivas N, et al., J. Immunol 191,17-23 (2013); Biswas S K et al. Nat. Immunol 11,889-896 (2010)). Indeed, the consequent intratumoral Teffector/Treg imbalance likely tilted the immune balance in the TME in favor of tumor deletion. These data demonstrate for the first time that the interdependence of a SAg (SEG-SEI) and MHCII (HLA-DQ8) enables modulation of the immune response and harnessing of SAgs tumoricidal response devoid of toxicity.

Methods and results of testing the anti-tumor effects and toxicity of SEG-SEI in HLA-DQ8, HLA-DR3 and C57BL/6 mice are shown below

### Methods

### Mice

All research performed, including animal and tissue collection, was conducted in accordance with the Animal Welfare Act and with the approval of the University of North Dakota's Institutional Animal Care and Usage Committee g(IACUC). Female HLA-DQ8 (DQA*0301/DQB*0302) and HLA-DR3 (HLA-DRA1*0101/HLA-DRB1*0301) tg breeding pairs were a gift from Dr. Chella David (Mayo Clinic, Rochester, Minn.) and their generation was described previously (Nabozny G H et al. J Exp Med 183, 27-37 (1996)). The HLA-DQ8 or HLA-DR3 phenotype was expressed on high proportion of PBL, spleen and lymph node cells (Mangalam A et al. J Immunol. 182, 5131-1539 (2009)). Breeding pairs of C57BL/6 mice were obtained from Jackson Laboratories (Bar Harbor, Me.). Mice were bred and maintained in specific pathogen-free conditions within the Center for Biological Research at the University of North Dakota.

### Cells

B16-F10 murine melanoma cells, Lewis lung carcinoma (LLC) and 4T1 mammary carcinoma were obtained from American Type Culture Collection (ATCC). All cells were maintained in complete Dulbecco's Modified Eagle's Medium (cDMEM) containing 10% heat inactivated FBS (Atlanta Biologicals) and 50 IU/ml Penicillin/Streptomycin (MP Biologicals) in T-75 flasks (CytoOne). Single cells were isolated from spleens and lymph nodes by passing organs through a 70 μm strainer (Falcon) with a 5 ml syringe plunger. All cells were washed with HBSS, RBCs were lysed with ACK lysis buffer for 5 min at 37° C. washed again with cDMEM and filtered through a 70 μm strainer.

### Superantigens

SEG and SEI, were produced in Escherichia coli M15 as His-tagged recombinant toxins and purified by affinity chromatography on a nickel affinity column as previously described (Thomas D et al. Infect Immun 77, 2043-2050 (2009)). Protein purity was verified by SDS-PAGE and TCR vBeta profiles established for each SE (Thomas D et al. supra 2009); Seo K S J Transl Med. 2010 Jan. 13; 8:2. doi: 10.1186/1479-5876-8-2). LPS was removed from toxin solutions by affinity chromatography (Detoxi-GEL endotoxin Gel, Pierce Rockford, USA). The QCL-1000 Limulus amebocyte lysate assay (CambrexBioWhittaker, Walkersville, USA) showed that the endotoxin content of the recombinant SAg solutions was less than 0.005 units/mL. All reagents were kept at either 4° C. or −20° C. and subject to no more than 2 freeze-thaw cycles.

Recombinant and biochemical preparation of SEG and SEI are given in U.S. 60/799,514, PCTUS05/022638, U.S. 60/583,692, U.S. 60/665,654, U.S. 60/626,159 which are incorporated by reference and their references in their entirety. Our most current methodology for manufacture of SEG and SEI with 98% purity is given as follows.

The prokaryotic expression cassette for the SEG was codon optimized and built synthetically and the gene was cloned into the pET24b(+) expression plasmid (kanamycin resistant) at the Ndel restriction site to avoid the addition of any tags onto the protein. Following the gene sequence, two STOP codons were inserted to prevent any read-through onto the His tag sequence present on the 3′ end of the MCS in the pET24b(+) vector. Signal sequences utilized by Staphylococcus aureus for protein activation and posttranslational shuttling were excluded leaving only the amino acid sequence of the mature peptide. The lyophilized DNA was suspended in 10 mM Tris/1 mM EDTA (pH 8) in a Class 100 BSC and then aliquoted at 200 ng/vial (20 ng/μl). The vials were frozen at −80° C.

Growth and Cell Lysis

1. The pET24b-SEG is transformed into BL21 (DE3) Veggie™ and expressed using an auto-induction medium (TBII derivative containing 0.4% lactose). The culture is grown for 20 hours at 30° C., 200 rpm, resulting in ˜20 g/L wet weight biomass (harvested by centrifugation).
2. The cells are resuspended in a solution containing 50 mM Tris-HCl, 5 mM EDTA, 10 mM BME, and 1% Triton X-100. The cell suspension is sonicated using a Branson Sonifier at a 50% Duty Cycle and an Output Power of 4 for a total sonication time of 1 min/gram.
3. The lysate is clarified by centrifugation at 15,000×g for 30 minutes. The resulting pellets are resuspended in the same solution and treated to a second round of sonication and clarification.
4. The lysates from each round of sonication are pooled prior to the first 5 chromatography step (approx. 1500 mg of soluble protein is extracted per liter of culture) Chromatography and Buffer Exchange
1. The clarified lysate is loaded onto a Q/SP Sepharose (mixed bed ion exchange) column and the load flow is collected for subsequent purification.
2. The load flow through from the Q/SP chromatography is diluted with a 50 mM MES, pH 5.5 buffer, 0.45 pM filtered, and loaded onto a SP Sepharose column. A gradient is run from 0-300 mM NaCl in 50 mM MES, pH 5.5 and fractions are collected, neutralized with Tris, and analyzed with SDS-PAGE.
3. Selected fractions from the CEX capture are pooled for further purification. The pooled post-CEX capture solution is diluted with an equal volume of 4.0 M (NH₄)₂SO₄, 50 mM Tris, pH 8.0, 0.45 pm filtered, and loaded onto an Octyl Sepharose Fast Flow column. A gradient is run from 2.0-1.0 M (NH₄)₂SO₄ and fractions are collected. Samples of each fraction are buffer exchanged and analyzed with SDS-PAGE.
4. Selected fractions from the HIC capture are pooled for further purification. The pooled fractions are diafiltered into 50 mM Tris, pH 7.0 on a 5 kDa Minimate system. The concentrated and buffer exchanged SEG is then loaded over a Q Sepharose Fast Flow column and the load flow is collected.
5. The LFT from the AEX void chromatography step is then ultrafiltered on a 25 kDa Minimate system for volume reduction prior to gel filtration.
6. The retentate from the ultrafiltration is 0.45 pm filtered and then loaded onto a Sephacryl S-200 HR gel filtration column equilibrated with IX PBS, pH 7.4.
7. All peaks are collected in fractions and analyzed with SDS-PAGE and silver staining. Selected fractions are pooled, 0.22 pm filtered, and samples transferred to Quality Control for analysis.

The references to amino acid sequences of SEG-SEI are incorporated by reference and their references in entirety as follows: SEG (Baba T. et al, Lancet 359, 1819-1827 (2002)); SEG (Jarraud S et al, J. Immunol. 166: 669-677 (2001)); SEI (Kuroda, M. et al. Lancet 357:1225-1240 (2001))

### Flow Cytometry

Cells were washed with HBSS, stained with Ghost Dye (TONBO) for viability, FC blocked (BioLegend) and stained for extracellular antigens. Cells were fixed and permeabilized using FoxP3 staining buffer kit (TONBO) for intracellular cytokine and transcription factor analysis. Fluorescence minus one (FMO) and single stained controls were used for gating and compensation. Gating strategies are indicated within each experiment. In general, doublets and cell debris were excluded, and only Ghost Dye negative cells were used for analysis. Samples were analyzed using a BD LSRII or Symphony A3 flow cytometer at the North Dakota Flow Cytometry and Cell Sorting (ND-FCCS) Core. Data were analyzed using FlowJo software.

### Assessment of T Cells

Splenocytes were isolated by passing mouse spleens through a 70 μm nylon strainer (Falcon) using the plunger from a 5 ml syringe. Cells were washed with HBSS (Gibco), lysed for 5 min in ACK lysis buffer and subsequently washed and resuspended in DMEM (Gibco) containing 10% heat inactivated FBS (Atlanta Biologicals) and Pen/Strep (1000 IU) (cDMEM) for downstream analysis. T cell proliferation was evaluated using Cell Proliferation Dye eFluor 450 (eBiosciences) dye. Briefly, splenocytes were stained with proliferation dye after which, 2×10⁵ cells/well were seeded in 96-well round-bottom tissue culture plates (Becton Dickinson) in cDMEM and stimulated with SAgs for 72 hrs (37° C., 5% CO₂ and humidity) in 200 μL volume. After 3 days, cells were processed for flow cytometry by using FC Block (BioLegend) and stained with 1:1000 dilution of TONBO ghost dye for 15 minutes. Cells were then washed with HBSS containing 2% FBS and stained for surface expression with 1:200 dilution of antibodies against CD3(17A2), CD4(RM4-5) CD8(53-6.7) and anti-CD127 (SB/199) (BioLegend) for 1 hr at 37° C. For detection of intracellular perforin (S16009A), granzyme B(QA16A02) and IFNγ(XMG1.2) (BioLegend) and FoxP3 (MF23) (TONBO), cells were fixed and permeabilized using Foxp3 staining buffer kit (TONBO) then stained for 1 hr at 37° C. Fluorescence minus one (FMO) and single stained controls were used for gating and compensation.

### Assessment of Tumor Cells

Tumors were isolated, rinsed with HBSS, minced with scissors and placed in 3 ml digestion solution containing 100 mg/ml type IV collagenase (Sigma) and 2,000 IU/ml type IV DNase (Sigma) in DMEM (Gibco) for 1 hr (37° C., 5% CO₂ and humidity) on a plate rocker. Tumor digests were passed through a 70 μm nylon strainer (Falcon) and cells were washed with HBSS (Gibco) and RBCs lysed for 5 min in ACK lysis buffer. Tumor cells were blocked using FC Block (BioLegend) and stained with 1:1000 dilution of TONBO ghost dye for 15 minutes. Peri of fluid. Cells were then washed with HBSS containing 2% FBS and stained with 1:200 dilution of antibodies against CD45(30-F11), CD3(17A2), CD4(RM4-5), CD8(53-6.7), CD44(IM7), CD62L(MEL-14), CD25(PC61), CD11b(M1/70), Gr-1(RB6-8C5) (TONBO and BioLegend) for 1 hr at 37° C. Cells were fixed and permeabilized using Foxp3 staining buffer kit (TONBO) then stained for 1 hr at 37° C. for granzyme B(QA16A02), IFNγ (XMG1.2) (BioLegend), FoxP3 (MF23) (TONBO). Fluorescence minus one (FMO) and single stained controls were used for gating and compensation. Gating strategies are indicated within each experiment.

### Assays of Cytokines and Chemokines

For measurement of cytokines in blood, samples were collected from the submandibular vein mice into EDTA tubes. Plasma cytokine concentrations were measured using LEGENDplex™ kit (BioLegend). Samples were processed via flow cytometry and analyzed via LEGENDplex software. For measurement of cytokines and chemokines in tumor tissue, tumors were weighed and lysed for 5 min using 2 mm stainless steel beads (Next Advance) and a Bullet Blender (Next Advance) in 300 μL HBSS. Lysates were centrifuged for 5 min at 5000×g. Supernatants were subjected to 1 freeze thaw cycle and cytokines and chemokines were measured using a TH cytokine cytometric bead array (Biolegend). Results are reported as (pg/ml)/mg of tumor tissue.

### Mixed Lymphocyte Assays

Mitomycin C-treated C57BL/6 splenocytes (red) were cocultured with splenocytes from HLA-DQ8 tg mice for 3 days at 37° C. in 5% CO₂. CD3+ T cell proliferation (green) was measured by dye dilution via flow cytometry and reported as percent proliferation. SEB was used as a positive control for T cell proliferation

### In Vivo Tumor Experiments

In vivo tumor experiments were carried out using an established model of peritoneal and omental tumor metastases for evaluation of intracavitary therapeutics (Kominsky S L et al. Int J Cancer. 94, 834-41 (2001); Gerber S A et al. Am J Pathol. 169, 1739-1752 (2006)). In the vaccination model, mice were injected with 1×10⁶ irradiated (15,000 rads) B16-F10 melanoma cells intraperitoneally (ip) in 100 μL HBSS (Life Technologies) on day −13. On day −7 and day −3, mice received 100 μL injections ip of SEG and SEI (50 μg each). Mice were challenged on day 0 with 2.5×10⁵ B16-F10 cells ip, evaluated daily and sacrificed when moribund. In the established tumor model, 2.5×10⁵ B16-F10 melanoma cells were injected ip in 100 μL PBS on day 0. On days 6 and 9 post tumor inoculation, mice were injected with SEG and SEI (50 μg each) ip in 100 μl of PBS. The same protocol was used to test the relative roles of T cell subsets in the anti-tumor response except individual groups of HLA-DQ8 tg mice were injected ip on days 2,4,7,9,12 with rat anti-mouse CD4+(200 μg), rat anti-CD8+(500 μg) and isotype matched rat IgG (300 μg) (BioXCell) in 100 μL PBS. Mice were evaluated daily and sacrificed when moribund.

### Radiation

Tumor cells were irradiated at 15,000 rads using Gammacell 3000 (MDS Nordion, Canada)

### Histology and Quantification of Tumor Density and Necrosis

Tumors were fixed with formalin and embedded in paraffin. Serial sections 5 μm thick were cut floated onto charged glass slides (Super-Frost Plus, Thermo Fisher Scientific) and dried overnight at 60° C. Sections were stained with hematoxylin and eosin (H&E). Slides were scanned at 1200 dpi and photographed as a TIFF image. The necrotic and viable areas of these sections were identified histologically with a Leitz Diaplan microscope and corroborated using ImageJ software. Necrotic tumor area was expressed as the percentage of pixels in the total area minus the area of the viable tumor cells regions in the tumor section (Jun J et al. Atherosclerosis. 209, 381-386 (2010); Kojima Y, et al. Nature 536, 86-90 (2016)). For determination of tumor density, all visible omental tumors were excised, and sections were stained, scanned, and photographed as TIFF images as described above. Tumor bearing areas were confirmed histologically and quantitated using the ImagJ software. Tumor density was expressed as total number of pixels in tumor bearing regions in each tumor section. Statistical analysis was based on a mean of 4 tumors per group.

### Data Analysis

One-way analysis of variance with Bonferroni's posttest and student's t test were performed where indicated. Kaplan Meier curves and Mantel-Cox Test were used to evaluate survival data. Statistical analysis was performed using GraphPad Prism software version 7.0a (La Jolla, Calif.).

### Results

SEG-SEI Induce Robust T Cell Proliferation and Production of T Cell Effector Molecules Along with Diminished Treg Differentiation in Splenocytes from Humanized MHCII Mice

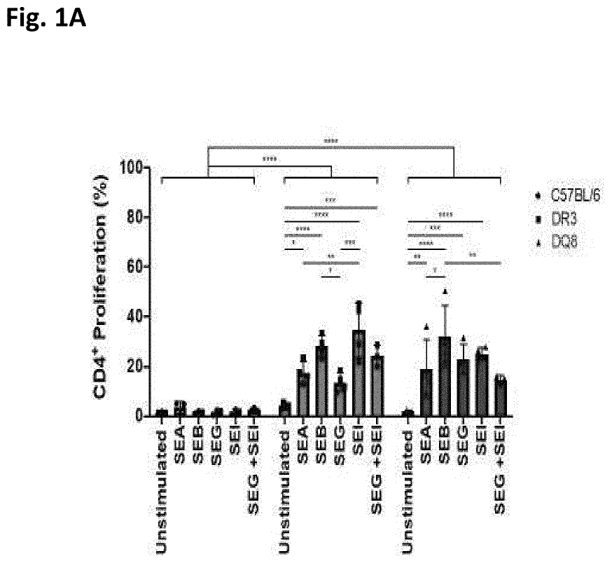

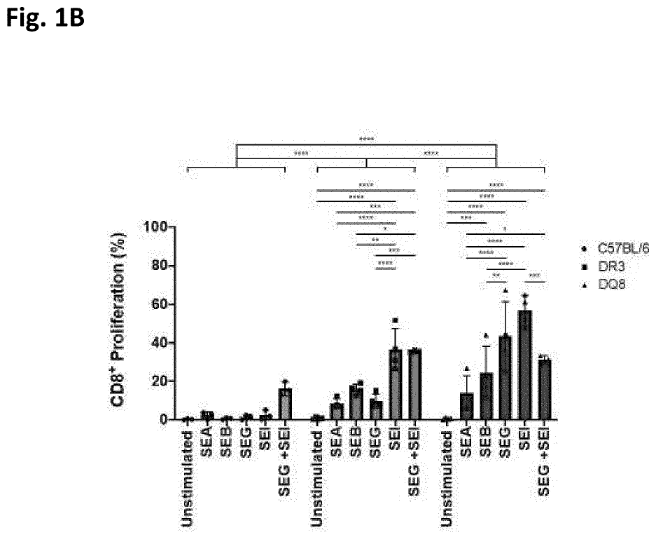

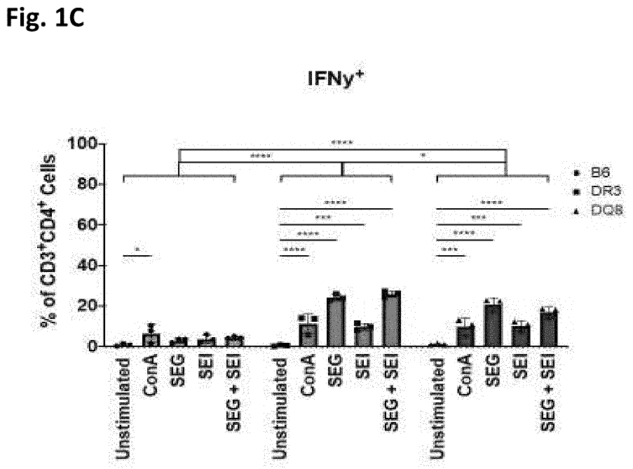

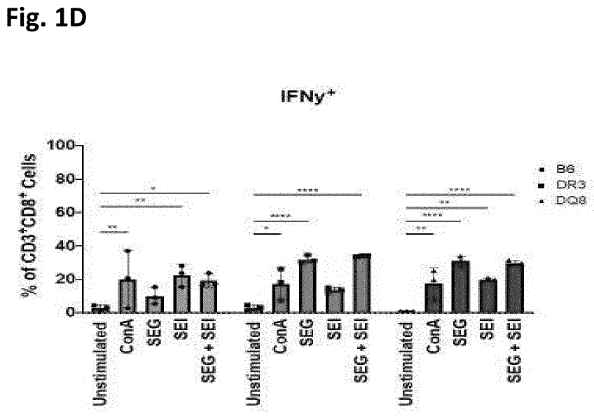

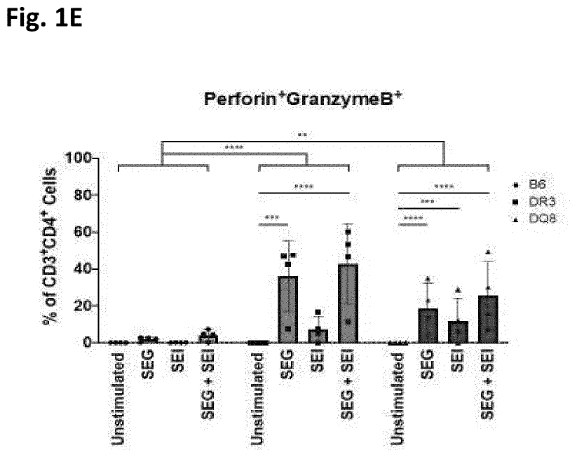

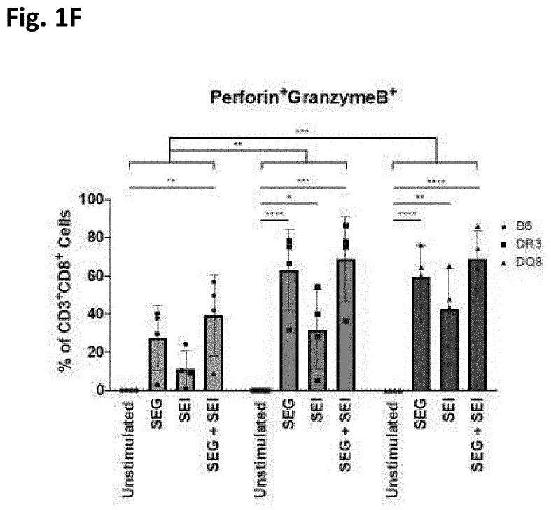

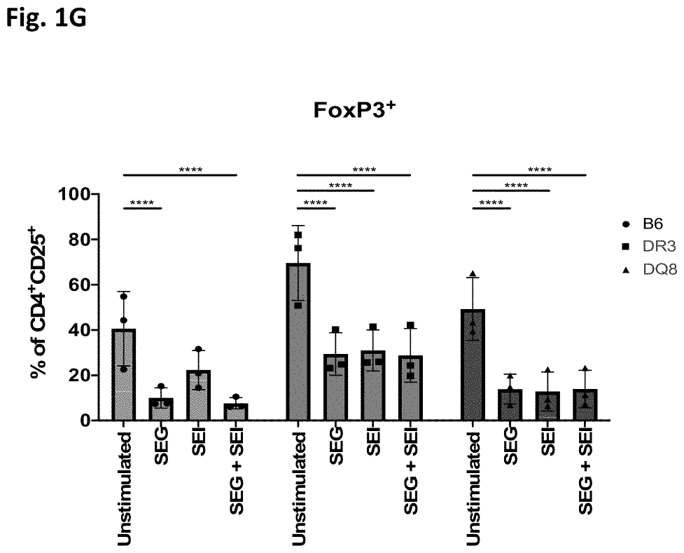

Although a fundamental feature of SAgs is the ability to induce robust T cell mitogenesis, individual SAgs often exhibit differential proliferative strength. In initial studies, we therefore compared the mitogenic strength of SEG, SEI, SEG-SEI to canonical SAgs SEB and SEA in naïve splenocytes from HLA-DQ8 and HLA-DR3 humanized mice along with C57BL/6 mice (MHCII I-A_{α}^{b} I-A_{β}^{b}). In all three strains, SEG, SEI, SEG-SEI, induced proliferation in CD4+ and CD8+ T cells comparable to that of canonical SEB and SEA (FIG. 1A,B). This finding affirmed the potent T cell mitogenic ability of SEG, SEI and SEG-SEI. Notably, T cell mitogenesis induced by all SEs in splenocytes from HLA-DQ8 and HLA-DR3 humanized mice was more potent relative to that of C57BL/6 splenocytes (FIG. 1A, FIG. 1B). The subdued proliferative response of C57BL/6 mice has been attributed to reduced binding affinities of SAg to murine MHCII alleles (Herman A et al. J Exp Med. 172,709-17 (1990)).

Next, we evaluated the ability of SEG, SEG and SEG-SEI to activate key T cell effector molecules, IFNγ and perforin-granzyme-B, in murine CD4+ and CD8+ T cells. SEG, SEI and SEG-SEI stimulated increased levels of IFNγ in CD4+ splenocytes from HLA-DQ8 tg and HLA-DR3 tg mice relative and C57BL/6 mice but comparable levels of IFNγ in CD8+ cells in all three strains (FIG. 1C, FIG. 1D). CD4+ and CD8+T splenocytes from both HLA-DQ8 tg and HLA-DR3 tg mice showed significantly higher levels of perforin-granzyme-B levels relative to splenocytes from C57BL/6 mice (FIG. 1E, FIG. 1F). We next evaluated the capacity of SEG-SEI to induce differentiation of CD4+CD25+Foxp3+(Tregs) in T cells from HLA-DQ8 tg, HLA-DR3 tg and C57BL/6 mice. In all three strains, SEG, SEI and SEG-SEI induced significant reductions of the Treg population relative to the untreated controls (FIG. 1G). Collectively, these findings indicate that SEG, SEI and SEG-SEI presented from a humanized HLA-DQ8 tg or HLA-DR3 tg platform possess robust superantigenic T cell proliferative function comparable to that of canonical SEA and SEB. SEG, SEI and SEG-SEI further demonstrate an ability to stimulate key T effector cell molecules, IFNγ and perforin-granzyme-B and subdue production of Tregs. The reduced proliferative and T effector responses noted in C57BL/6 splenocytes accords with the reported heightened efficiency of SAgs to stimulate T cells when they are presented from a human MHCII platform in humanized mice (Welcher B C et al. supra (2002). Rajagopalan G et al. supra (2002).

### SEG/SEI-Treated HLA-DQ8 tg Mice Display Long-Term Survival of Established B16-F10 Melanoma and Resistance to Tumor Rechallenge

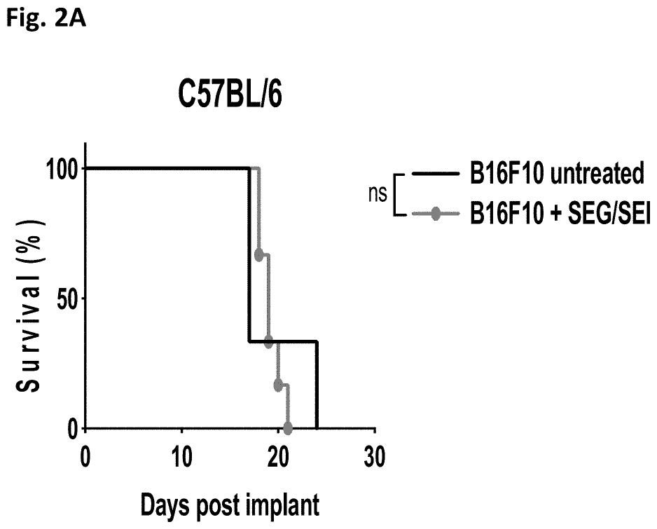

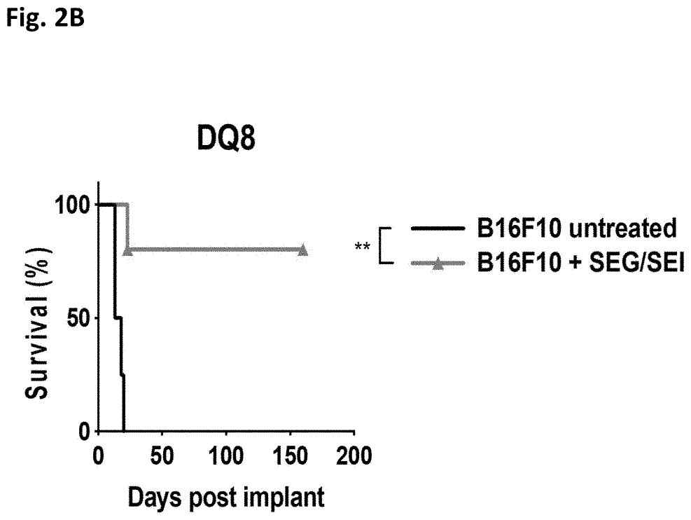

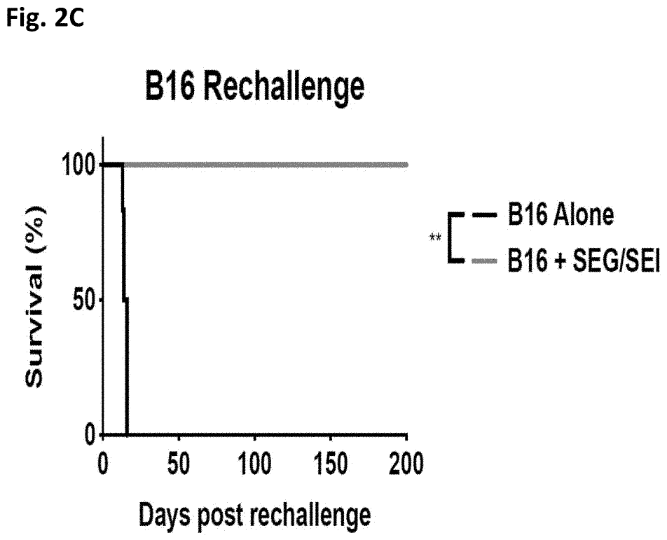

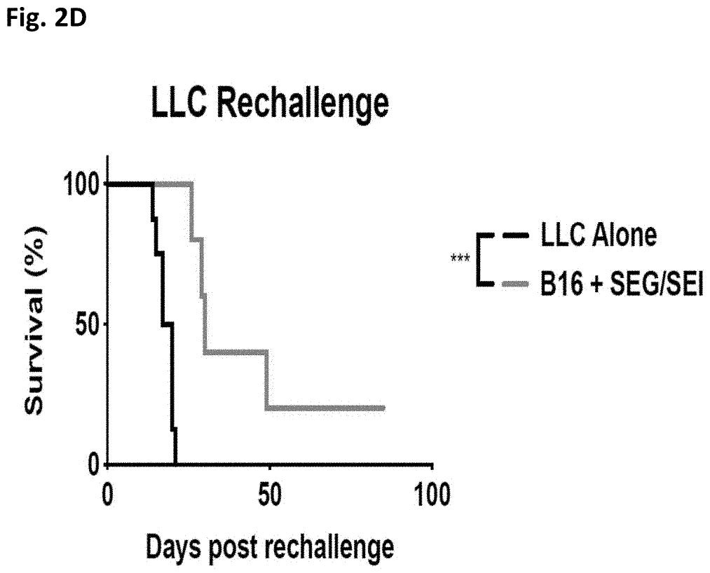

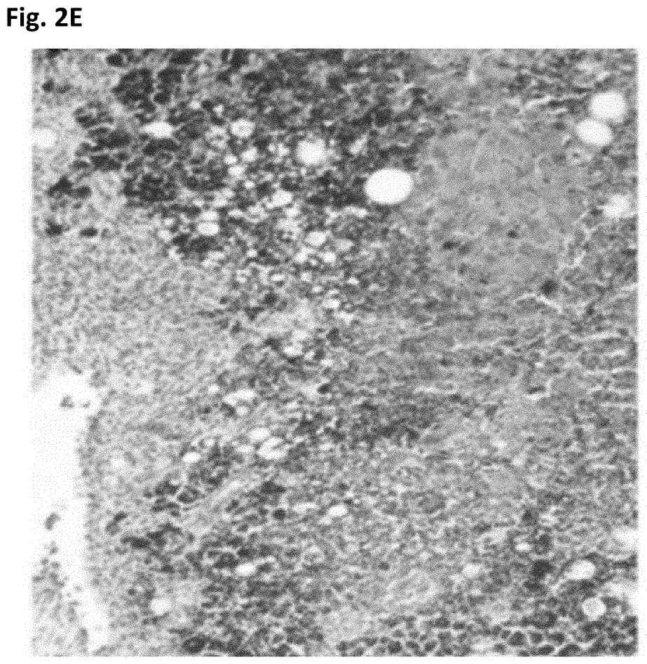

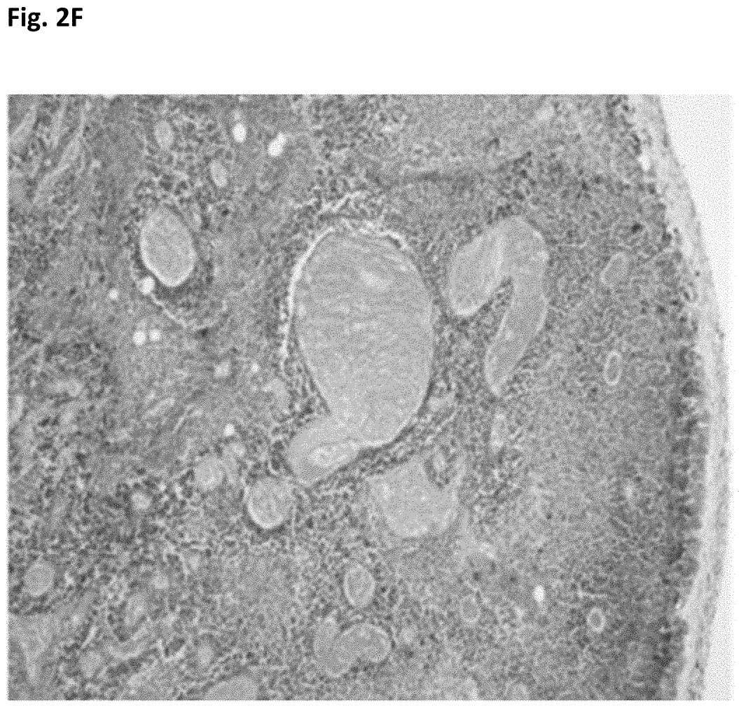

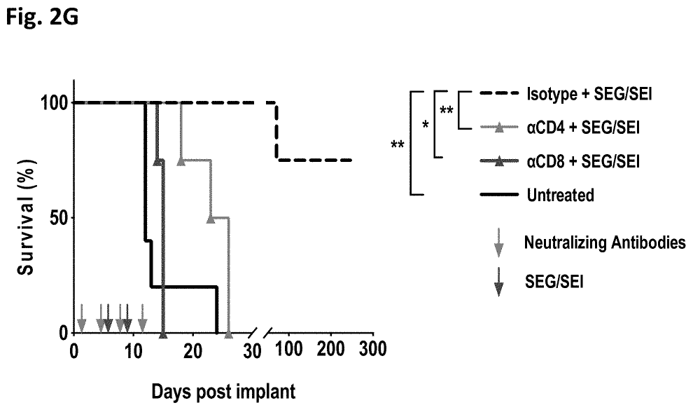

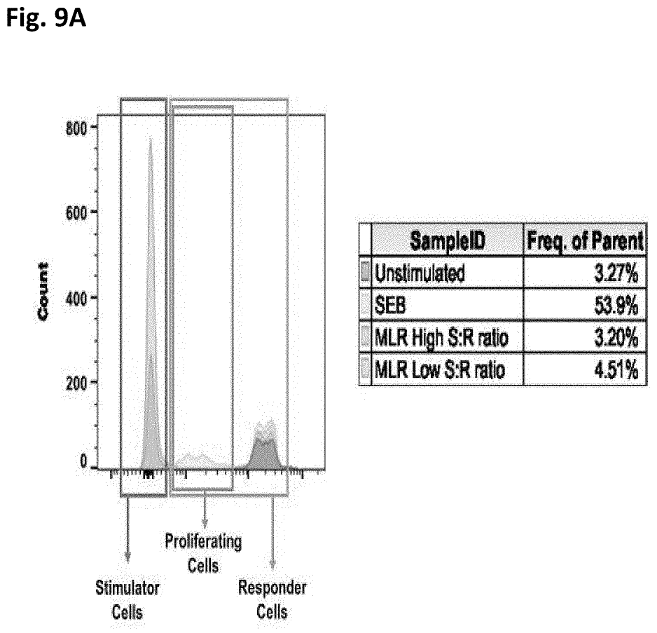

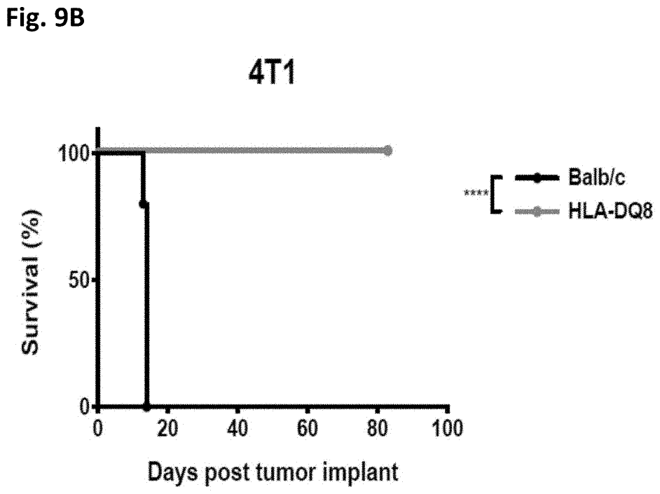

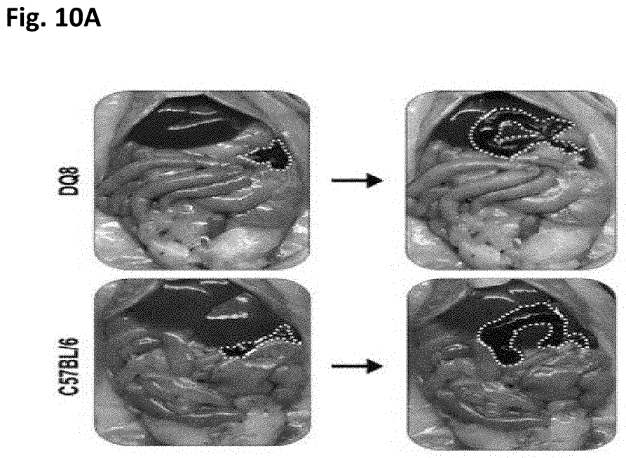

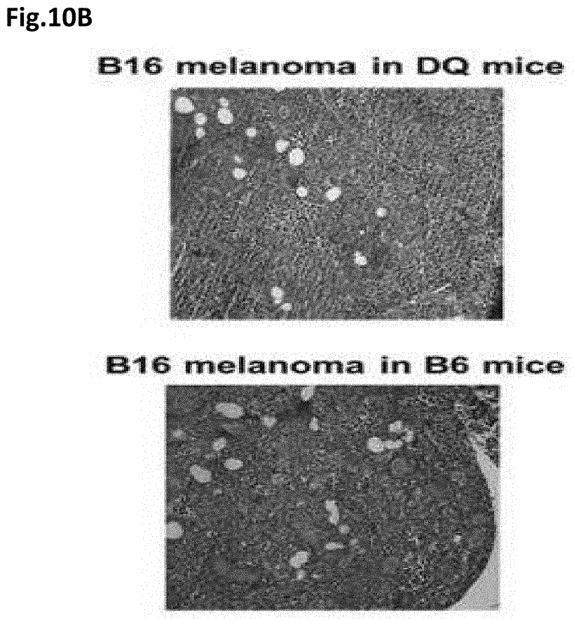

In preliminary studies, we examined the genetic compatibility of HLA-DQ8 tg and C57BL/6 mice. As expected from their shared a genetic background (Nabozny G H et al. J Exp Med 183, 27-37 (1996)) mitomycin treated C57BL/6 splenocytes failed to stimulate HLA-DQ8 cells in mixed lymphocyte culture (FIG. 9A). Predictably, the B16-F10 melanoma indigenous to the C57BL/6 mice was lethal in both strains 20 days after receiving a tumorigenic dose of B16-F10 melanoma cells (FIG. 2A FIG. 2,B). HLA-DQ8 tg mice showed that they were not immunologically impaired since they eradicated the genetically unrelated 4T1 carcinoma of Balb/C origin (FIG. 9B). We also established that within six days after injection of B16-F10 melanoma cells both C57BL/6 and HLA-DQ8 tg mice exhibited omental metastases of comparable degree along with established histopathologic stroma and angiogenesis (FIG. 10). Taken together, these data indicate genetic compatibility of the C57BL/6-derived B16-F10 melanoma in HLA-DQ8 tg mice and further show that the B16-F10 tumor was broadly established in both strains at the time SEG-SEI treatment was initiated.

Next, we examined the ability of SEG-SEI to prolong survival of mice with established B16-F10 melanoma in HLA-DQ8 tg and C57BL/6 mice. Mice received a tumorigenic dose of B16-F10 cells on day 0 and were subsequently injected with 50 μg each of SEG and SEI on days 6 and 9. As predicted from their subdued T cell proliferative response and diminished ability to generate T effector molecules in vitro, SEG-SEI did not prolong survival in C57BL/6 mice (FIG. 2A). By contrast, 9 of 11 HLA-DQ8 tg mice treated with SEG-SEI survived for 160 days whereas all untreated HLA-DQ8 tg controls died by day 20 after tumor inoculation (FIG. 2B). To determine whether the long-term surviving mice could mount a specific anti-tumor memory response against the melanoma, 5 of the 9 surviving mice were rechallenged with a tumorigenic dose of Lewis lung carcinoma (LLC) cells and the remaining 4 mice were rechallenged with a tumorigenic dose B16-F10 melanoma cells. The group rechallenged with B16F10 melanoma survived for an additional 200 days (FIG. 2C). By contrast, 4 of 5 mice challenged with LLC died within 50 days (FIG. 2D). Histology of untreated and SEG-SEI-treated tumors in HLA-DQ8 tg mice 13 days after tumor inoculation showed disseminated islands of melanoma cells in the untreated samples whereas the SEG-SEI treated sections displayed broadly propagated tumor necrosis engulfing the core and tumor periphery (FIG. 2E,Fug 2F). SEG-SEI treatment therefore evoked an acute tumoricidal response corroborated histologically followed by long-term survival in HLA-DQ8 tg mice. The resistance of these mice to melanoma but not LLC rechallenge suggests that the SEG-SEI treatment also produced tumor specific, long-term anti-tumor memory that enabled sustained survival.

### Anti-CD4 or Anti-CD8 Specific Antibodies Abrogate the Anti-Tumor Effect of SEG-SEI Against Established Melanoma

Having shown above that SEG-SEI could stimulate both CD4+ and CD8+ T cells in splenocytes from HLA-DQ8 tg mice and induce long term survival against established melanoma, we next determined the relative roles of CD8+ and CD4+ T cells in the tumoricidal response. B16-F10 melanoma was implanted in HLA-DQ8 tg mice on day 0 and SEG-SEI injected ip on days 6 and 9. Individual groups received anti-CD4 or anti-CD8 specific antibodies while controls received isotype matched IgG2b antibodies. Results shown in FIG. 2G demonstrate attenuated survival of the SEG-SEI treated mice receiving anti-CD4 or anti-CD8 antibodies whereas 80% of mice receiving isotype control antibodies survived for 200 days after tumor inoculation. These data indicate a role for both CD4+ and CD8+ T cells in the SEG-SEI-induced anti-tumor response in HLA-DQ8 tg mice. SEG-SEI-treated HLA-DQ8 tg mice show increased T effector cells along with minimal Tregs and myeloid cells in the TME

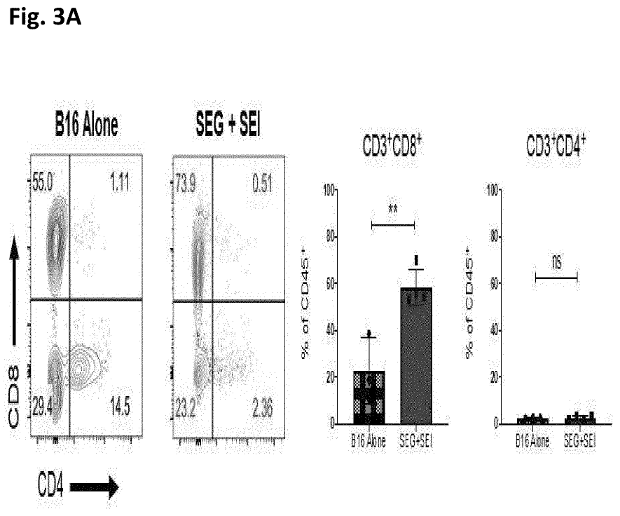

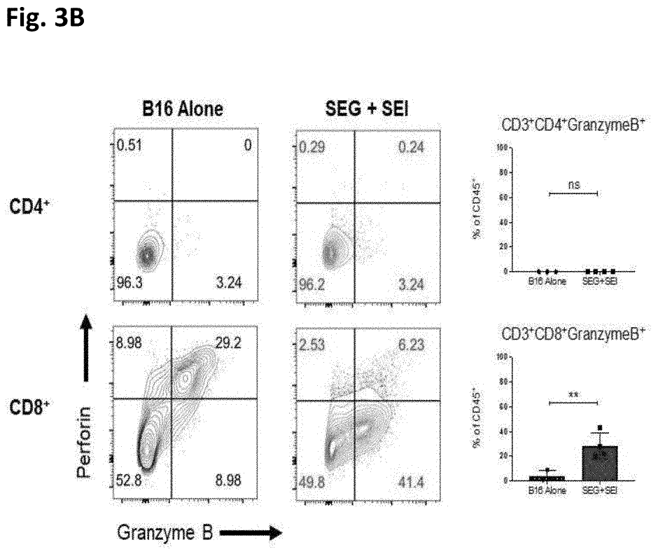

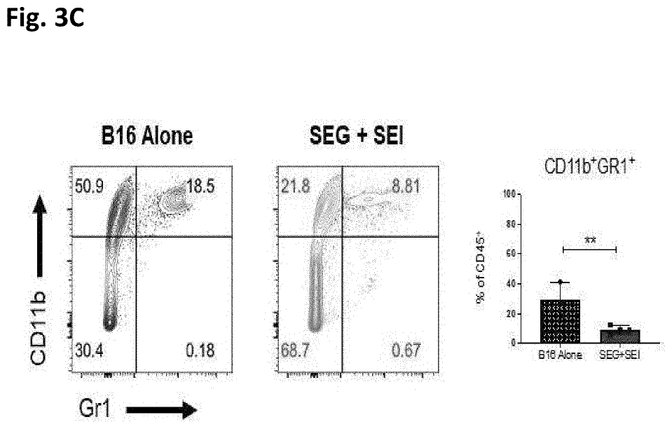

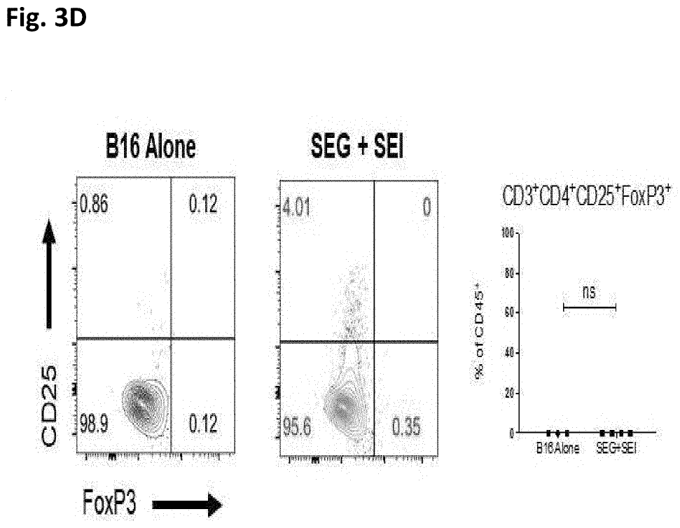

We next examined the quantity of CD4+, CD8+ T cells, Teffector cells, Tregs and myeloid cells in tumors of mice treated with SEG-SEI in comparison to untreated controls. HLA-DQ8 tg mice were inoculated with B16-F10 melanoma on day 0 and treated with SEG-SEI on days 6 and 9. Tumors were removed and analyzed on day 13. Untreated tumor bearing HLA-DQ8 tg mice served as controls. Tumors from SEG-SEI-treated HLA-DQ8 tg mice showed a significant increase of CD8+ and granzyme-B+T effector cells relative to tumors from untreated HLA-DQ8 tg mice (FIG. 3A, FIG. 3B). By contrast myeloid cells in tumors from SEG-SEI-treated HLA-DQ8 tg mice were significantly reduced while Tregs were unchanged compared to the untreated controls (FIG. 3C, FIG. 3D). Ratios of Teffector cells to Tregs or Teffector cells to myeloid cells in SEG-SEI-treated HLA-DQ8 tg mice versus untreated HLA-DQ8 tg controls were significantly increased (FIG. 3 legend). Collectively, these findings point to CD8+T effector cells as key mediators of the acute tumoricidal response and suggest that their anti-tumor effectiveness in the TME may be enhanced by the minimal presence of myeloid cells and suppressive Tregs.

### SEG-SEI-Treated HLA-DQ8 tg Mice Display Increased TH-1 Cytokines in Serum and TME

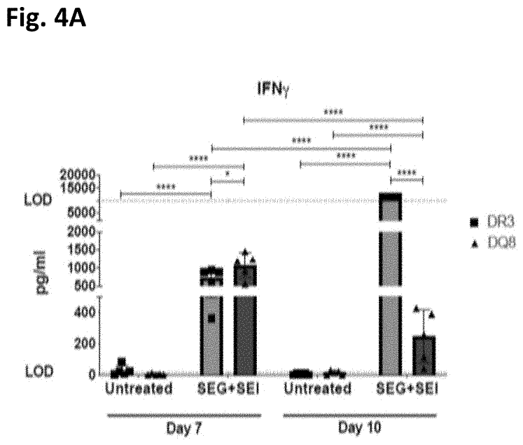

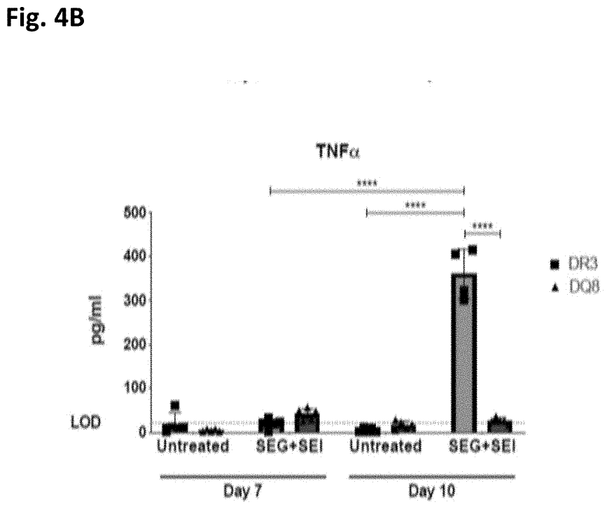

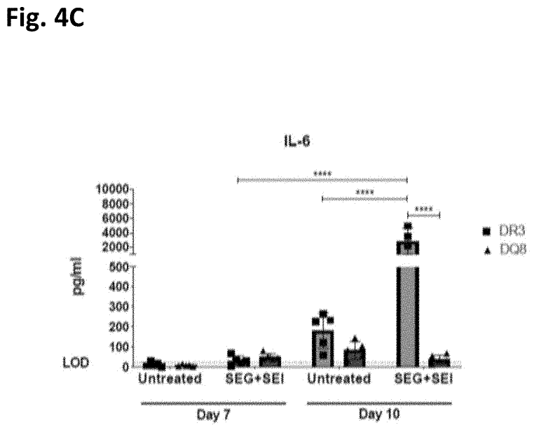

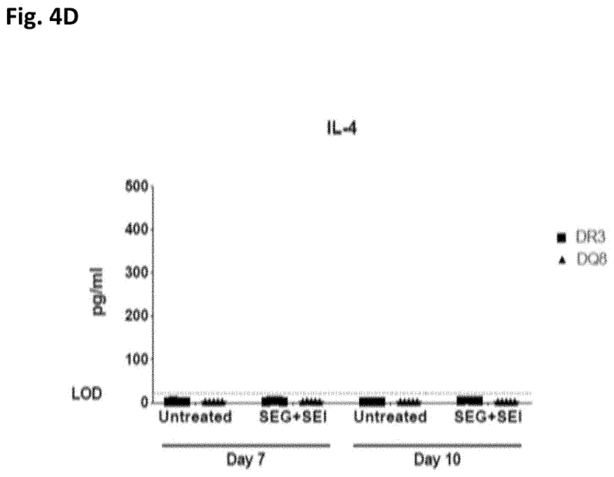

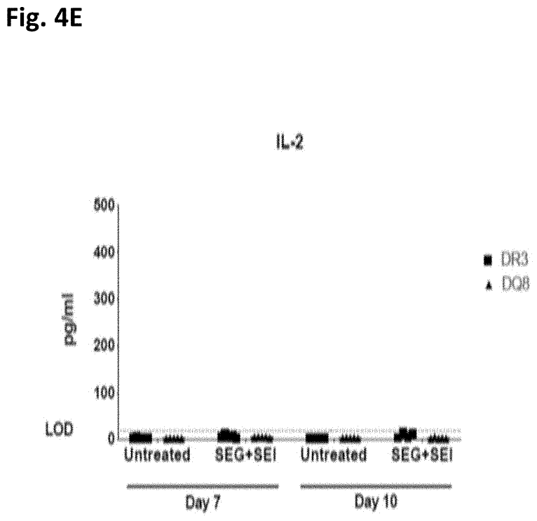

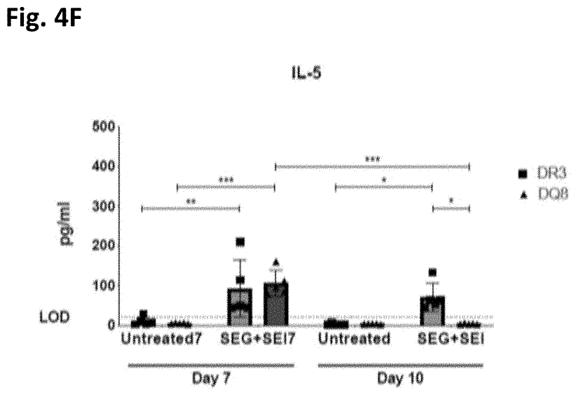

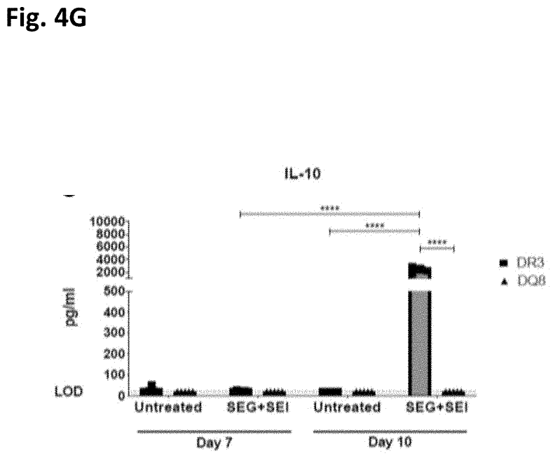

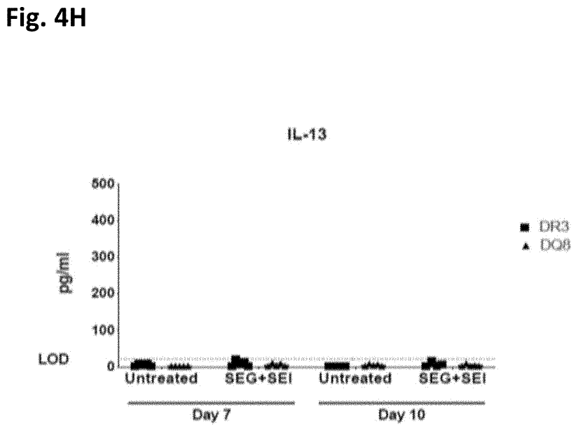

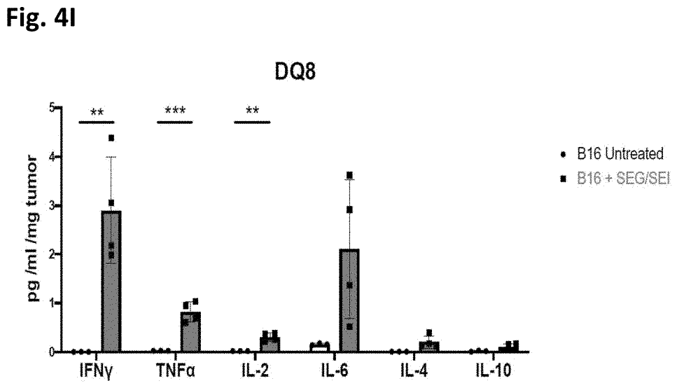

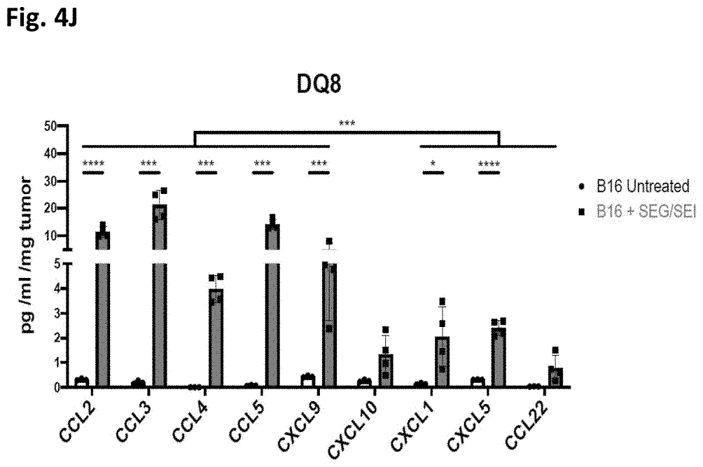

Having shown that SEG-SEI activated IFNγ in vitro, we now examined the effect SEG-SEI treatment on production of IFNγ and other cytokines in vivo in both serum and the TME. HLA-DQ8 tg mice were inoculated with a tumorigenic dose of B16-F10 cells on day 0 and subsequently injected with 50 μg of SEG and SEI on days 6 and 9. Serum levels of TH-1 and TH-2 cytokine levels were evaluated on 24 hours after each treatment on days 7 and 10. On day 7, HLA-DQ8 tg mice exhibited a surge of IFN with minimal changes in TNFa levels FIG. 4A, FIG. 4B). On day 10, however, following the second SEG-SEI treatment, the IFNγ response was attenuated along with minimal levels of TNFa and IL-6 (FIG. 4A, FIG. 4B, FIG. 4C). By contrast TH-2 cytokines IL-4, IL-10 and IL-13 were not significantly elevated in HLA-DQ8 tg mice after either SEG-SEI treatment (FIG. 4E, FIG. 4F, FIG. 4H). Notably, the sharp increase in IFNγ was not accompanied by any evident toxicity in HLA-DQ8 tg mice. Comparative toxicity and cytokine responses in identically-treated HLA-DR3 tg mice are detailed below.

Next, we analyzed B16-F10 tumors obtained from SEG-SEI-treated HLA-DQ8 tg mice for the presence of cytokines on day 13 following tumor inoculation. HLA-DQ8 tg mice received a tumorigenic dose B16-F10 melanoma cells on day 0 and were treated with SEG-SEI, 50 μg of each, on days 6 and 9. Controls were inoculated with tumor but received no treatment. SEG-SEI-treated HLA-DQ8 tg mice showed significant increases in IFNγ, TNFα and IL-6 in the TME not seen in untreated controls (FIG. 4I). TH-2 cytokines IL-4 and IL-10 were not significantly altered in SEG-SEI treated HLA-DQ8 tg mice relative to untreated mice. In view of the established tumoricidal properties of IFNγ, its ability upregulate MHCII expression on melanoma cells and prime cytolytic CD8+ T cells, IFN likely contributed significantly to the acute tumoricidal response. TNFα also noted in the TME has been shown to synergize with IFNγ in tumor cytolysis. The presence of IFNγ and TNFα together with the T effector cells in the TME, not seen in untreated controls, suggests that these are the major cytokine contributors to the acute tumoricidal effect in SEG-SEI-treated HLA-DQ8 tg mice. SEG-SEI treated HLA-DQ8 tg mice display increased T effector cell chemokines in the TME

A hypothetical barrier to successful immunotherapy is inadequate recruitment of activated T cells and T effector cells into the tumor sites. An optimal chemokine profile in the melanoma TME may be critical for recruitment of activated T cells. Homing of effector T cells to tumors is thought to depend in part on chemokines. To evaluate intratumoral chemokines, HLA-DQ8 tg mice were inoculated with a tumorigenic dose of B16-F10 melanoma and treated with SEG-SEI, 50 μg of each, on days 6 and 9. Controls received tumor cells but no treatment. Tumors were removed on day 16 and evaluated for chemokines. SEG-SEI-treated mice showed significant increases in CCL2, CCL3, CCL5, CXCL9, CXCL10, CXCL9, CXCL10 chemokines relative to the untreated controls (FIG. 4). Interestingly, Gajewski et al., identified the very same chemokine expression profile in treatment-responsive metastatic melanomas and further showed that each of them was efficient at recruiting activated T cells (Harlin H et al. Cancer Res. 69, 3077-3085 (2009)). Each of these chemokines displayed a significantly higher level than CXCR5 and CXCL1 and CCL22 chemokines that have been shown to recruit PMN/MDSCs, PMNs or Tregs respectively (FIG. 4J) (Soler-Cardona et al. J Invest Dermatol. 138,1627-1635 (2018); Sawant K V, Sci. Rep. 6, 33123; doi: 10.1038/srep33123 (2016); Bischoff L, J Immunol. 194, 3054-3064 (2015)). Collectively, these findings suggest that the intratumoral chemokine grouping identified here contributed to the recruitment T effector cells in the TME following SEG-SEI treatment as noted in FIG. 3A, FIG. 3B.

### SEG-SEI-Treated HLA-DQ8 tg Mice Recruit Teffector Cells to the Site of Tumor Injection

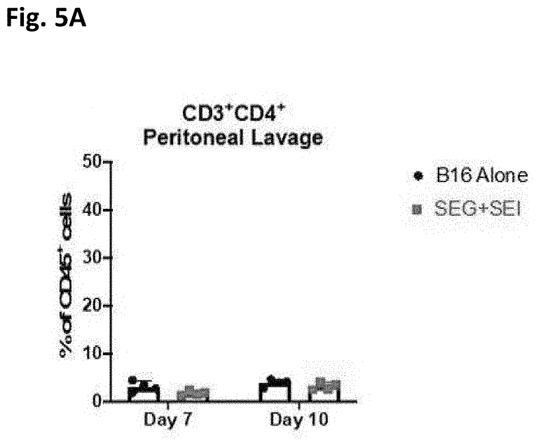

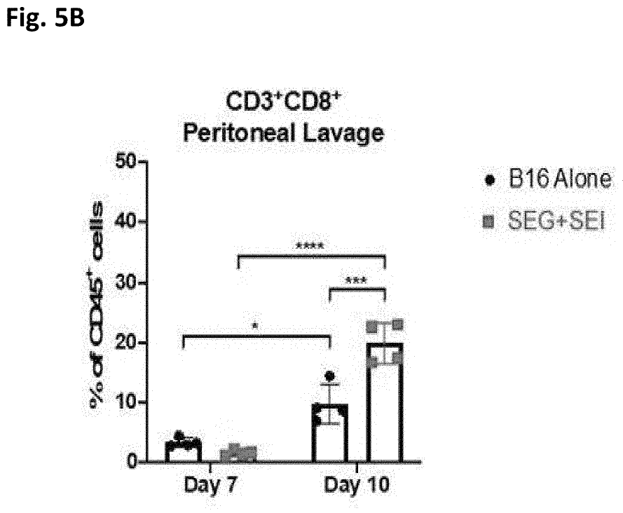

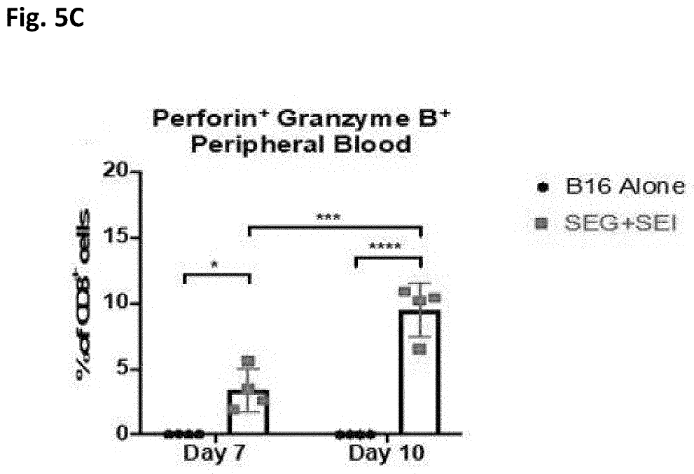

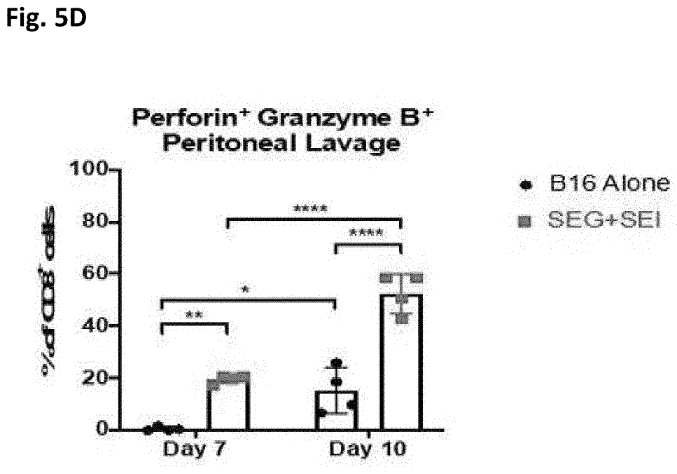

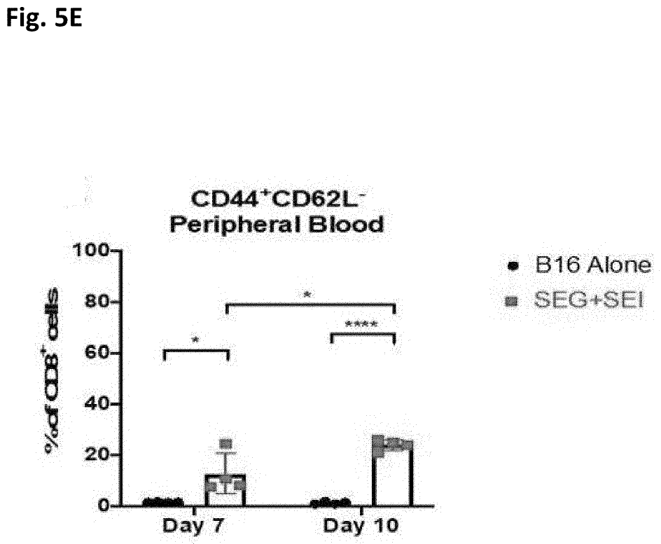

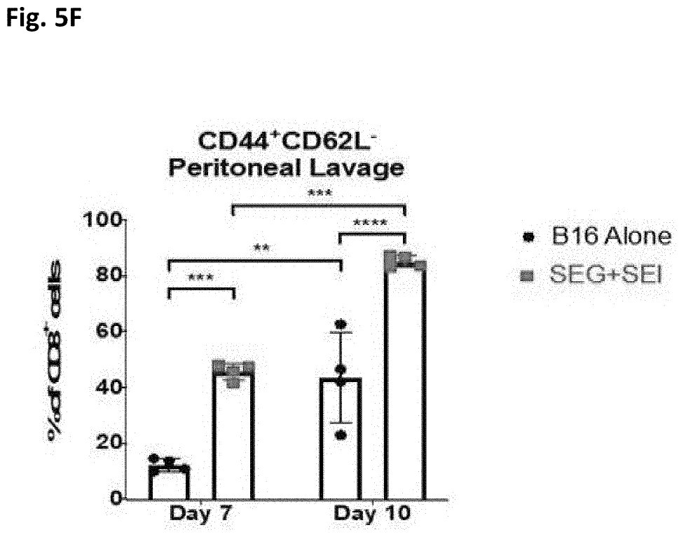

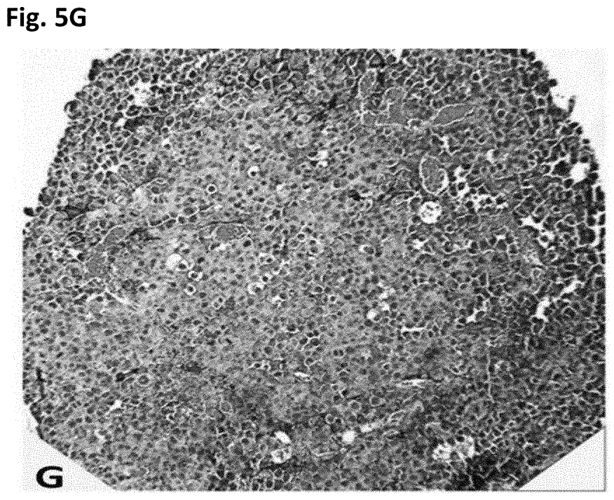

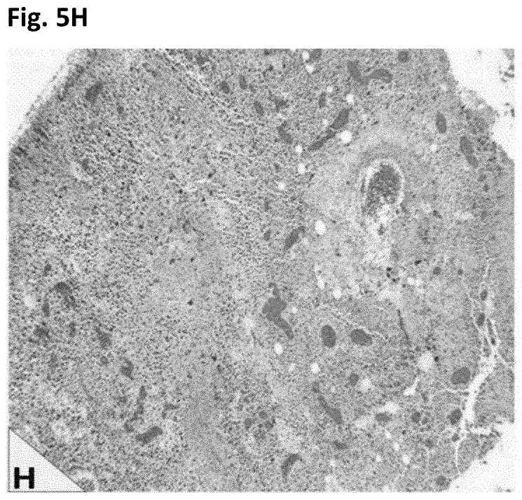

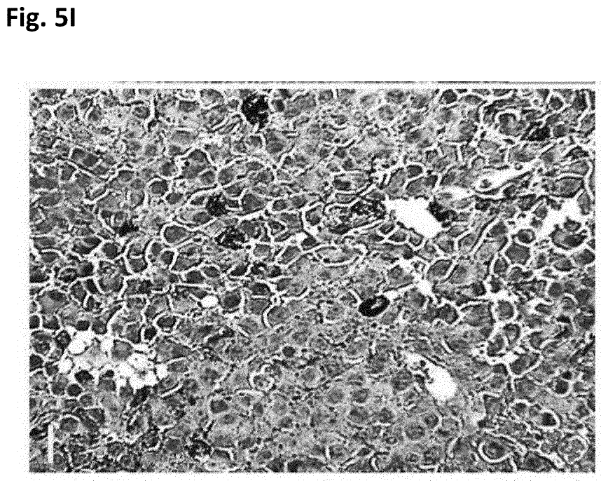

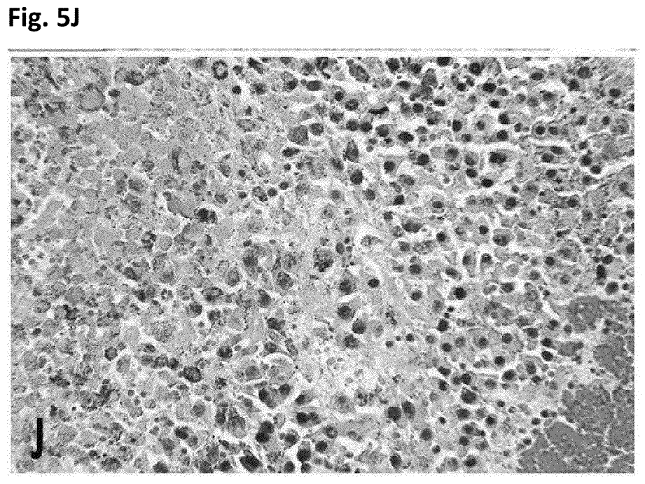

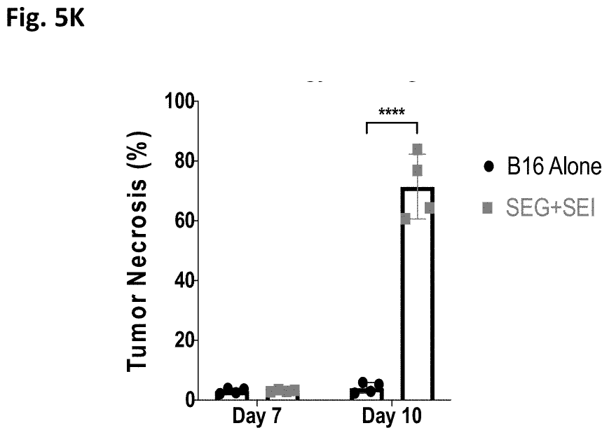

While only a small number of CTLs are generally able to infiltrate the tumor site, following SAg inoculation activated T cells are known to traffic to the SAg injection site (Slaney C Y et al. Cancer Res. 74, 7168-7174 (2014); DeGrendele H C et al. Science. 278,672-5(1997)). We therefore hypothesized that SEG-SEI administration to the intraperitoneal tumor site could induce the recruitment of T effector cells to that site. To test this concept, HLA-DQ8 tg mice were inoculated with a tumorigenic dose of B16-F10 melanoma cells and treated with SEG-SEI ip on days 6 and 9. Twenty four hours after each treatment on days 7 and 10, tumors were removed and both peritoneal lavage and peripheral blood were assessed for the presence of T effector cells. Controls were injected with B16-F10 cells on day 0 with sham treatments of PBS on days 6 and 9. On days 7 and 10, SEG-SEI treated mice showed significant absolute and incremental increases in the number of activated (CD44+CD62L−) and CD8+ effector (granzyme-perforin+) T cells in both peripheral blood and peritoneal lavage compared to untreated controls (FIG. 5A, FIG. 5B, FIG. 5C, FIG. 5D, FIG. 5E, FIG. 5F). Analysis of omental tumor in SEG-SEI treated mice on day 10 showed a significantly greater degree of tumor necrosis relative to the untreated control encompassing a mean of 70% of the tumor area (FIG. 5G, FIG. 5H, FIG. 5I). These findings indicate that SEG-SEI injection recruited T effector cells to the site of tumor injection. Such activated T cells in the peritoneal cavity are known to traffic rapidly to tumor niches in the omentum and likely account in part for their appearance in TME of omental tumors undergoing a tumoricidal response as shown in FIG. 3A FIG. 3,B. The concurrent increase of Teffector cells in both the peripheral blood and peritoneal T effector cells induced by SEG-SEI suggests that the peritoneal effector T cell population may have originated, in part, from a T effector cell pool in the peripheral blood.

Vaccination with Irradiated Melanoma Cells Followed by SEG-SEI Affords Protection of HLA-DQ8 tg Mice from Living Melanoma Cell Challenge

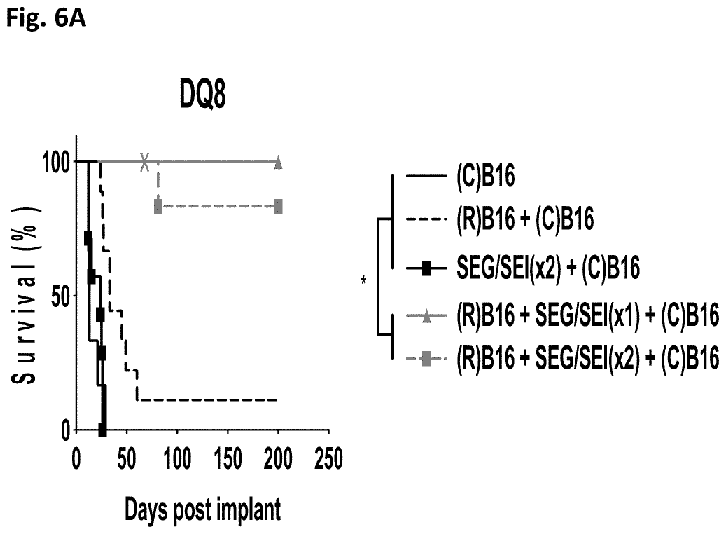

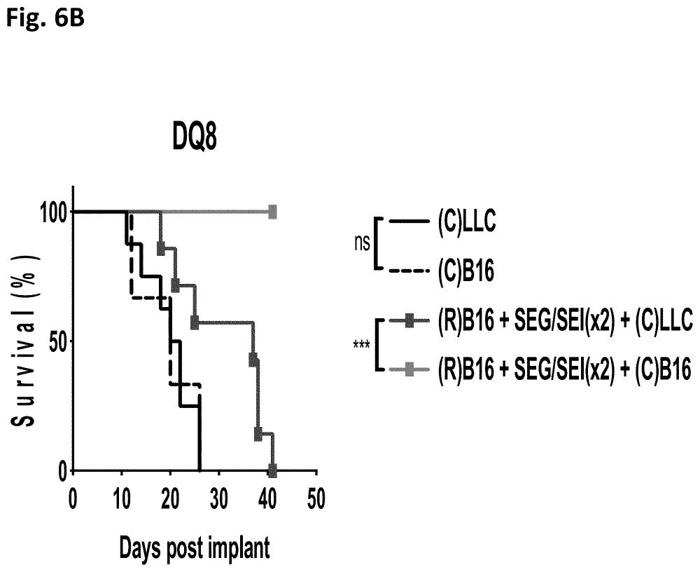

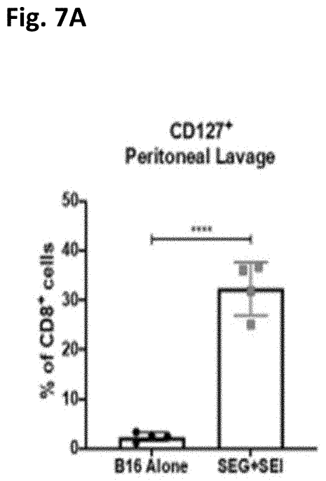

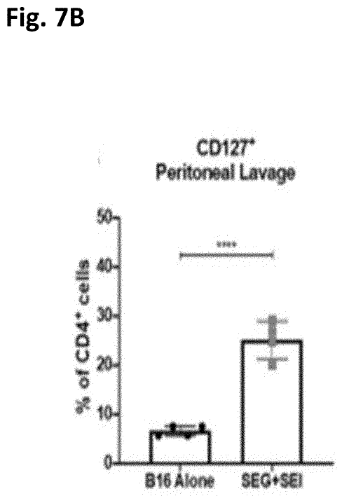

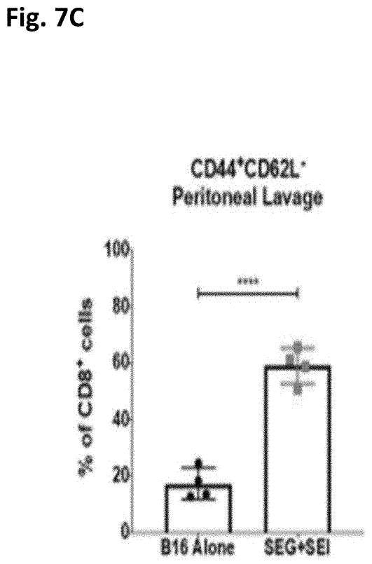

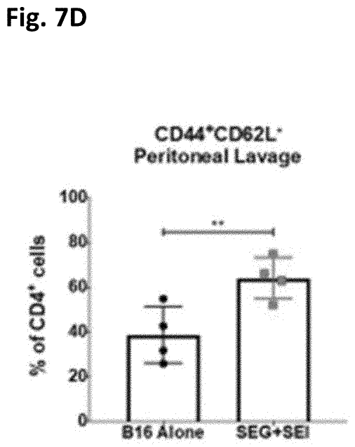

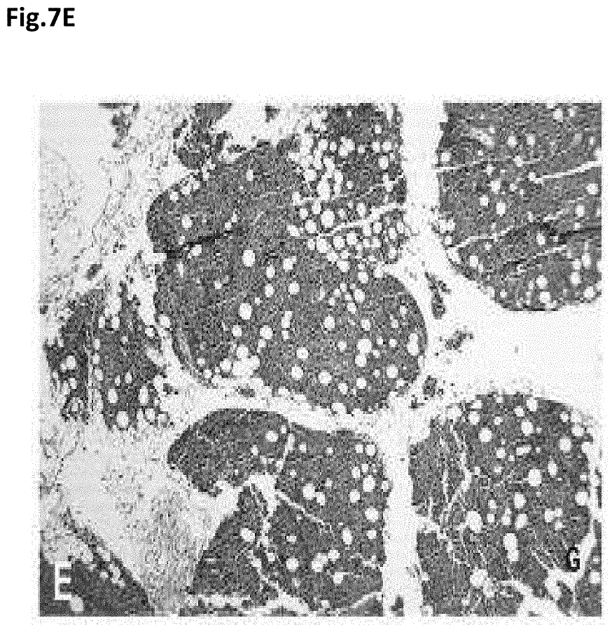

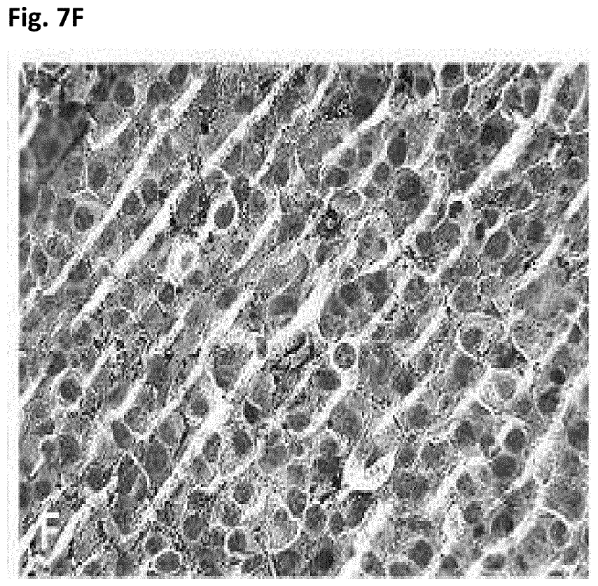

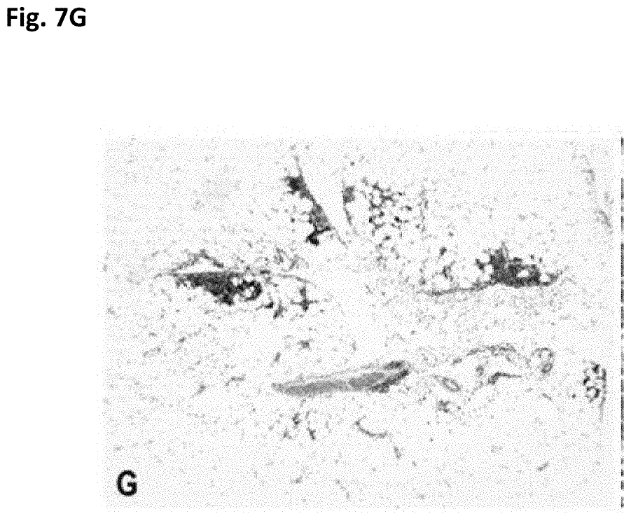

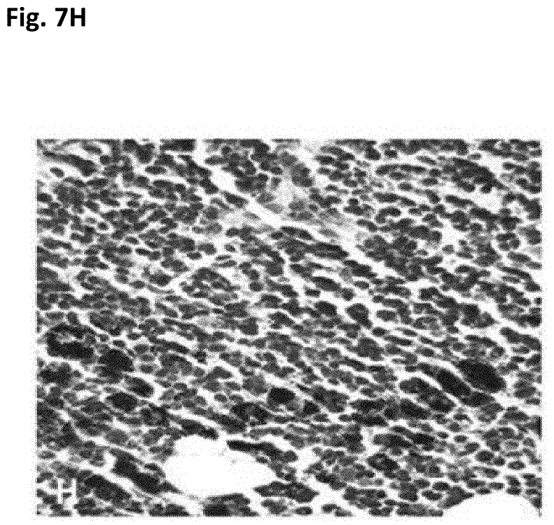

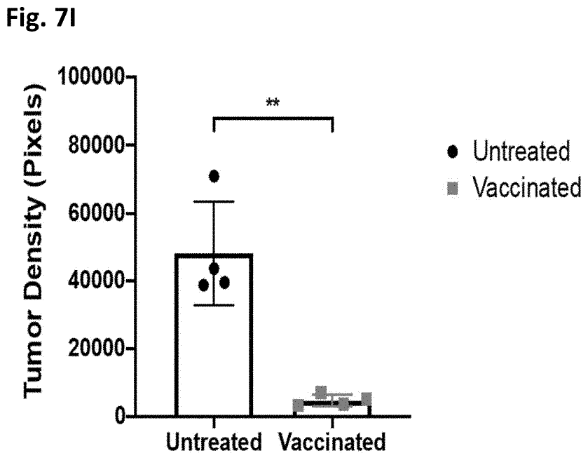

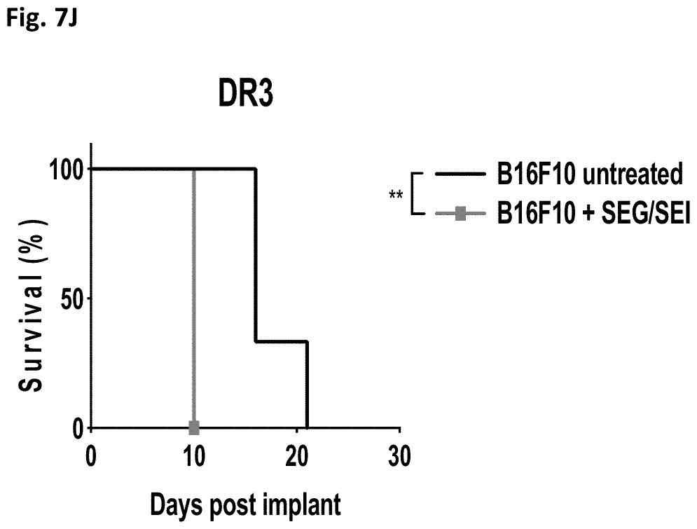

We next wished to determine whether SEG-SEI could enhance an antitumor immune response to radiation-inactivated B16-F10 melanoma cells and confer protection from live B16-F10 challenge in HLA-DQ8 tg mice. Ionizing radiation is a powerful mutagen and cancer cells that survive these treatments often carry new mutations. These mutations frequently occur in genes encoding proteins involved in DNA-repair mechanisms and those encoding multiple cell-cycle regulators suggesting that some of them will be immunogenic (Lhuillier C, et al., Genome Med. 2019 Jun. 20; 11(1):40. doi: 10.1186/s13073-019-0653-7). To test this hypothesis, Mice were vaccinated with 10⁶ irradiated B16-F10 melanoma cells on day −13 and subsequently treated with 50 μg of SEG and SEI on day −7 and day −3 and challenged with 2.5×10⁵ viable B16-F10 cells on day 0. Mice receiving sham treatment, irradiated tumor cell vaccination or SEG-SEI alone served as controls. HLA-DQ8 tg mice receiving irradiated tumor cell vaccination followed by SEG-SEI survived significantly longer than controls (FIG. 6A). We next determined whether the anti-tumor protection induced by vaccination combined with SEG-SEI treatment could protect surviving mice against a second tumor challenge. HLA-DQ8 tg mice that survived for more than 60 days after tumor implant were rechallenged with a tumorigenic dose of melanoma cells (FIG. 6A). All of these mice remained tumor-free for the succeeding 140 days (FIG. 6A). These results show that vaccination followed by SEG-SEI administration elicits anti-melanoma protection against primary and secondary B16-F10 melanoma challenge. Next, we examined the anti-tumor specificity of the response induced by vaccination with irradiated B16-F10 cells and SEG-SEI treatment. HLA-DQ8 tg mice were vaccinated with 1×10⁶ irradiated (15,000 rads) B16-F10 cells on day −13, treated with SEG and SEI, 50 μg of each, on days −7 and −3 and challenged with 2.5×10⁵ live B16-F10 or Lewis lung carcinoma cells on day 0. All mice pretreated with irradiated B16-F10 cells plus SEG-SEI and challenged with living B16-F10 cells demonstrated survival to day 40 whereas none of the mice similarly vaccinated but challenged with Lewis lung carcinoma (LLC) cells survived beyond 40 days (FIG. 6B). Hence, the protection induced by the vaccination regimen of irradiated tumor cells plus SEG-SEI was tumor specific. To better understand the basis of the recall response of vaccinated mice to tumor challenge HLA-DQ8 tg mice were vaccinated with 1×10⁶ irradiated B16-F10 cells on day −13, treated with SEG and SEI, 50 μg of each, on days −7 and −3 and challenged with 2.5×10⁵ live B16-F10 cell on day 0. On day 40, they were rechallenged 2.5×10⁵ live B16-F10 cells and 6 days later peritoneal lavage and omental tumor were examined. Unvaccinated HLA-DQ8 treated inoculated with tumor cells and sacrificed 6 days later served as controls, Peritoneal lavage of vaccinated and tumor rechallenged mice showed significant elevations of a mixture of activation and memory T cells CD127 and CD44+CD62L− T cells respectively relative to the unvaccinated control (FIG. 7A, FIG. 7B, FIG. 7C, FIG. 7D). Histologic analysis of all omental tumor deposits in vaccinated mice and untreated control mice showed striking differences (FIG. 7 E, FIG. 7F, FIG. 7G, FIG. 7H). Whereas the control tumor cells were situated in discrete tumor nodules with well developed stromal matrix (FIG. 7E, FIG. 7F), tumor cells in the vaccinated mice were positioned amid large aggregates of mononuclear cells devoid of organized tumor matrix or distinct tumor nodules (FIG. 7G, FIG. 7H). These aggregates likely constitute omental milky spots infiltrated by tumor cells. Vaccinated mice also showed a total omental tumor density that was 11-fold less than the untreated control (FIG. 7I). Vaccination therefore not reduced the number of omental tumor cells and constrained the formation of an organized tumor nodules relative to untreated controls. Collectively, these data indicate that vaccination with radiation-inactivated B16-F10 cells plus SEG-SEI affords tumor specific, long-term anti-tumor protection against the B16-F10 challenge. The reduction in omental tumor density was corroborated histologically. Protection appeared to be mediated, in part, by a population of activated and memory T cells. Relative to untreated control, omental tumor cells in vaccinated mice were reduced in number, localized amid large mononuclear cell aggregates and they failed to form discrete tumor nodules.

### SEG-SEI Toxicity in HLA-DQ8 tg Versus HLA-DR3 tg Mice

SEG-SEI treatment in HLA-DQ8 tg mice was well tolerated. There were no acute deaths and mice showed no signs of inanition, anorexia, or disability during or after treatment. Arthritis susceptible HLA-DQ8 tg mice (Bradley D S, et al. J. Immunol. 161, 5046-5053 (1998)). treated with SEG-SEI also exhibited no clinical signs of arthritic joint swelling, ambulatory impairment, polydipsia or weight loss during 360 days of observation. We further tested SEG-SEI in transgenic HLA-DR3 tg mice that share an identical genetic background to the HLA-DQ8 tg strain but express the human HLA-DR3 allotype as opposed to the HLA-DQ8 allele. HLA-DR3 tg mice were injected with a tumorigenic dose of B16-F10 cells on day 0 and subsequently treated with 50 μg of SEG and SEI on days 6 and 9. On day 10, following the second SEG-SEI injection, all HLA-DR3 tg mice developed toxic shock and death (FIG. 7J). Their early death after treatment precluded assessment of an anti-tumor response in these mice. To understand the basis of this toxic response we examined serum cytokine levels in HLA-DR3 tg mice after each of the SEG-SEI treatments. For comparison, we also studied cytokine responses of HLA-DQ8 tg mice which did not show toxic shock after SEG-SEI treatment. On day 7, 24 hours after the first SEG-SEI injection, HLA-DR3 tg and HLA-DQ8 tg mice exhibited comparable increases in IFNγ levels with minimal changes in TNFα levels (FIG. 4A, FIG. 4B). However, on day 10, 24 hours after a second SEG-SEI treatment, HLA-DR3 tg mice exhibited a surge in serum levels of IFNγ, TNFa, IL-6 not seen in HLA-DQ8 tg mice (FIG. 4A, FIG. 4B, FIG. 4C. By contrast, TH-2 cytokines IL-4 and IL-13 were not significantly elevated in HLA-DR3 tg mice (FIG. 4E, FIG. 4F, FIG. 4H). TNFα, IFNγ and IL-6 have been implicated as causal factors in SAg-induced toxic shock and provide a basis for the toxic shock in HLA-DR3 tg mice (Miethke T et al. J. Exp Med 175, 91-98 (1992) Tilahun A Y et al. PLoS One. 2011 Feb. 3; 6(2):e16764. doi: 10.1371/journal.pone.0016764). The absence of toxicity in HLA-DQ8 tg mice following SEG-SEI treatment in HLA-DQ8 tg mice may be ascribed to their muted TNFα, IFNγ, IL-6 response and suggests a key role of the HLA-DQ8 allele in constraining the SEG-SEI-mediated cytokine production and toxic shock seen in HLA-DR3 tg mice.

### Functional Fragments, Homologues and Derivatives of Superantigens

The present invention contemplates, the use of homologues of wild type SEG and SEI that have the requisite biological activity of stimulating T cell mitogenesis via the v region of the TCR at low doses to be useful in accordance with the invention. The are produced recombinantly by methods essentially identical to that of the wild type SEG and SEI as described herein.

Thus, in addition to native proteins and nucleic acid compositions described herein, the present invention encompasses functional derivatives, among which homologues are preferred. By “functional derivative” is meant a “fragment,” “variant,” “mutant,” “homologue,” “analogue,” or “chemical derivative. Homologues include fusion proteins, chimeric proteins and conjugates that include a SAg portion fused to or conjugated to a fusion partner polypeptide or peptide. A functional derivative retains at least a portion of the biological activity of the native protein which permits its utility in accordance with the present invention. For superantigens, such biological activity includes stimulation of T cell proliferation and/or cytokine secretion, stimulation of T cell-mediated cytotoxic activity, as a result of interactions of the SAg composition with T cells preferably via the TCR Vβ or Vα region.

A “fragment” refers to any shorter peptide. A “variant” refers to a molecule substantially similar to either the entire protein or a peptide fragment thereof. Variant peptides may be conveniently prepared by direct chemical synthesis of the variant peptide, using methods well-known in the art.

A homologue refers to a natural protein, encoded by a DNA molecule from the same or a different species. Homologues, as used herein, typically share at least about 50% sequence similarity at the DNA level or at least about 18% sequence similarity at the amino acid level, with a native protein.

An “analogue” refers to a non-natural molecule substantially similar to either the entire molecule or a fragment thereof

A “chemical derivative” contains additional chemical moieties not normally a part of the peptide. Covalent modifications of the peptide are included within the scope of this invention. Such modifications may be introduced into the molecule by reacting targeted amino acid residues of the peptide with an organic derivatizing agent that is capable of reacting with selected side chains or terminal residues.

A fusion protein comprises a native protein, a fragment or a homologue fused by recombinant means to another polypeptide fusion partner, optionally including a spacer between the two sequences. Preferred fusion partners are antibodies, Fab fragments, single chain Fv fragments. Other fusion partners are any peptidic receptor, ligand, cytokine, domain (“ECD”) of a molecule and the like.

The recognition that the biologically active regions of the proteins, for example, are substantially homologous, i.e., that the sequences are substantially similar, enables prediction of the sequences of synthetic peptides which will exhibit similar biological effects in accordance with this invention.

The following terms are used in the disclosure of sequences and sequence relationships between two or more nucleic acids or polypeptides: (a) “reference sequence”, (b) “comparison window”, (c) “sequence identity”, (d) “percentage of sequence identity”, and (e) “substantial identity”

As used herein, “reference sequence” is a defined sequence used as a basis for sequence comparison. A reference sequence may be a subset or the entirety of a specified sequence, for example, as a segment of a full-length cDNA or other polynucleotide sequence, or the complete cDNA or polynucleotide sequence. The same is the case for polypeptides and their amino acid sequences.

As used herein, “comparison window” includes reference to a contiguous and specified segment of a polynucleotide or amino acid sequence, wherein the sequence may be compared to a reference sequence and wherein the portion of the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides or amino acids in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of nucleotide and amino acid sequences for comparison are well-known in the art. For comparison, optimal alignment of sequences may be done using any suitable algorithm, of which the following are examples:
(a) the local homology algorithm (“Best Fit”) of Smith and Waterman, Adv. Appl. Math. 2: 482 (1981);
(b) the homology alignment algorithm (GAP) of Needleman and Wunsch, J. Mol. Biol. 48: 443 (1970); or
(c) a search for similarity method (FASTA and TFASTA) of Pearson and Lipman, Proc. Natl. Acad. Sci. 85 2444 (1988);

In a preferred method of alignment, Cys residues are aligned. Computerized implementations of these algorithms, include, but are not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG) (Madison, Wis.). The CLUSTAL program is described by Higgins et al. Gene 73:237-244 (1988); Higgins et al., CABIOS 5:151-153 (1989); Corpet et al. Nuc Acids Res 16:881-90 (1988); Huang et al. CABIOS 8:155-65 (1992), and Pearson et al. Methods in Molecular Biology 24:307-331 (1994)).

A preferred program for optimal global alignment of multiple sequences is PileUp (Feng and Doolittle J Mol Evol 25:351-360 (1987) ) which is similar to the method described by Higgins et al., 1989, supra).

The BLAST family of programs which can be used for database similarity searches includes: NBLAST for nucleotide query sequences against database nucleotide sequences; XBLAST for nucleotide query sequences against database protein sequences; BLASTP for protein query sequences against database protein sequences; TBLASTN for protein query sequences against database nucleotide sequences; and TBLASTX for nucleotide query sequences against database nucleotide sequences. See, for example, Ausubel et al., eds., Current Protocols in Molecular Biology, Chapter 19, Greene Publishing and Wiley-Interscience, New York (1995) or most recent edition. Unless otherwise stated, stated sequence identity/similarity values provided herein, typically in percentages, are derived using the BLAST 2.0 suite of programs (or updates thereof) using default parameters. Altschul et al., Nuc Acids Res. 25:3389-3402 (1997).

As is known in the art, BLAST searches assume that proteins can be modeled as random sequences. However, many real proteins comprise regions of nonrandom sequence which may include homopolymeric tracts, short-period repeats, or regions rich in particular amino acids. Alignment of such regions of “low-complexity” regions between unrelated proteins may be performed even though other regions are entirely dissimilar. A number of low-complexity filter programs are known that reduce such low-complexity alignments. For example, the SEG (Wooten et al. Comput. Chem. 17:149-163 (1993) and XNU (Claverie et al. Comput. Chem, 17:191-201 (1993) low-complexity filters can be employed alone or in combination.

As used herein, “sequence identity” or “identity” in the context of two nucleic acid or amino acid sequences refers to the residues in the two sequences which are the same when aligned for maximum correspondence over a specified comparison window. It is recognized that when using percentages of sequence identity for proteins, a residue position which is not identical often differs by a conservative amino acid substitution, where a substituting residue has similar chemical properties (e.g., charge, hydrophobicity, etc.) and therefore does not change the functional properties of the polypeptide. Where sequences differ in conservative substitutions, the % sequence identity may be adjusted upwards to correct for the conservative nature of the substitution and be expressed as “sequence similarity” or “similarity” (combination of identity and differences that are conservative substitutions). Means for making this adjustment are well-known in the art. Typically this involves scoring a conservative substitution as a partial rather than as a full mismatch, thereby increasing the percentage sequence identity. Thus, for example, where an identical amino acid is given a score of “1” and a non-conservative substitution is given a score of “0” zero, a conservative substitution is given a score between 0 and 1. The scoring of conservative substitutions is calculated, e.g., according to the algorithm of Meyers et al. CABIOS 4:11-17 (1988) as implemented in the program PC/GENE (Intelligenetics, Mountain View, Calif., USA).

As used herein, “percentage of sequence identity” refers to a value determined by comparing two optimally aligned sequences over a comparison window, wherein the portion of the nucleotide or amino acid sequence in the comparison window may comprise additions or deletions (i.e., gaps) as compared to the reference sequence (which lacks such additions or deletions) for optimal alignment, such as by the GAP algorithm (supra). The percentage is calculated by determining the number of positions at which the identical nucleotide or amino acid residue occurs in both sequences to yield the number of matched positions, dividing that number by the total number of positions in the window of comparison and multiplying the result by 100, thereby calculating the percentage of sequence identity.

The term “substantial identity” of two sequences means that a polynucleotide or polypeptide comprises a sequence that has at least 60%, preferably at least 70%, more preferably at least 80%, even more preferably at least 90%, and most preferably at least 95% sequence identity to a reference sequence using one of the alignment programs described herein using standard parameters. Values can be appropriately adjusted to determine corresponding identity of the proteins encoded by two nucleotide sequences by taking into account codon degeneracy, amino acid similarity, reading frame positioning, etc.

One indication that two nucleotide sequences are substantially identical is if they hybridize to one other under stringent conditions. Because of the degeneracy of the genetic code, a number of different nucleotide codons may encode the same amino acid. Hence, two given DNA sequences could encode the same polypeptide but not hybridize under stringent conditions. Another indication that two nucleic acid sequences are substantially identical is that the polypeptide encoded by the first nucleic acid is immunologically cross reactive with the polypeptide encoded by the second nucleic acid. Clearly, then, two peptide or polypeptide sequences are substantially identical if one is immunologically reactive with antibodies raised against the other. A first peptide is substantially identical to a second peptide, if they differ only by a conservative substitution. Peptides which are “substantially similar” share sequences as noted above except that nonidentical residue positions may differ by conservative substitutions.

Thus, in one embodiment of the present invention, the Lipman-Pearson FASTA or FASTP program packages (Pearson, W. R. et. al., 1988, supra; Lipman, D. J. et al. Science 227:1435-1441 (1985)) in any of its older or newer iterations may be used to determine sequence identity or homology of a given protein, preferably using the BLOSUM 50 or PAM 250 scoring matrix, gap penalties of −12 and −2 and the PIR or SwissPROT databases for comparison and analysis purposes. The results are expressed as z values or E ( ) values. To achieve a more “updated” z value cutoff for statistical significance, preferably corresponding to a z value >10 based on the increase in database size over that of 1988, in a FASTA analysis using the equivalent 2001 database, a significant z value would exceed 13.

A more widely used and preferred methodology determines the percent identity of two amino acid sequences or of two nucleic acid sequences after optimal alignment as discussed above, e.g., using BLAST. In a preferred embodiment of this approach, a polypeptide being analyzed for its homology with native protein is at least 20%, preferably at least 40%, more preferably at least 50%, even more preferably at least 60%, and even more preferably at least 70%, 80%, or 90% as long as the reference sequence. The amino acid residues (or nucleotides) at corresponding positions are then compared. Amino acid or nucleic acid “identity” is equivalent to amino acid or nucleic acid “homology”.

In a preferred comparison of a putative polypeptide or peptide homologue polypeptide and a native protein, the percent identity between two amino acid sequences is determined using the Needleman and Wunsch alignment algorithm (incorporated into the GAP program in the GCG software package (available at the URL www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between the encoding nucleotide sequences is determined using the GAP program in the GCG software package (also available at above URL), using a NWSgapdna.CMP matrix and a gap weight of 40, 50, 60, 70, or 80 and a length weight of 1, 2, 3, 4, 5, or 6. In another embodiment, the algorithm of Meyers et al. supra (incorporated into the ALIGN program, version 2.0), is implemented using a PAM120 weight residue table, a gap length penalty of 12 and a gap penalty of 4.

The wild-type (or native) SAg-encoding nucleic acid sequence or the SAg protein sequence can further be used as a “query sequence” to search against a public database, for example, to identify other family members or related sequences. Such searches can be performed using the NBLAST and XBLAST programs, supra (see Altschul et al. (1990) J. Mol. Biol. 215:403-10). BLAST nucleotide searches can be performed with the NBLAST program, score=100, wordlength=12 to identify nucleotide sequences homologous to native SAgs. BLAST protein searches can be performed with the XBLAST program, score=50, wordlength=3 to identify amino acid sequences homologous to identify polypeptide molecules homologous to a native SAg. To obtain gapped alignments for comparison purposes, Gapped BLAST can be utilized as described in Altschul et al. (1997, supra). Default parameters of XBLAST and NBLAST can be found at the NCBI website (www.ncbi.nlm.nih.gov)

Using the FASTA programs and method of Pearson and Lipman, a preferred SAg homologue is one that has a z value >10. Expressed in terms of sequence identity or similarity, a preferred SAg homologue for use according the present invention has at least about 20% identity or 25% similarity to native SAg. Preferred identity or similarity is higher. More preferably, the amino acid sequence of a homologue is substantially identical or substantially similar to a native protein molecule as those terms are defined above.

One group of substitution variants (also homologues) are those in which at least one amino acid residue in the peptide molecule, and preferably, only one, has been removed and a different residue inserted in its place. Deletion and addition variants are also homologues if they satisfy the structural and functional criteria set forth herein with respect to their parent or native molecules. For a detailed description of protein chemistry and structure, see Schulz, G. E. Principles of Protein Structure Springer-Verlag, New York, 1978, and Creighton, T. E., Proteins: Structure and Molecular Properties, W. H. Freeman & Co., San Francisco, 1983, which are hereby incorporated by reference. The types of substitutions which may be made in the protein or peptide molecule of the present invention may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, kg, Lys;
4. Large aliphatic, nonpolar residues: Met, Leu, Ile, Val (Cys); and
5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Tyr, because of its hydrogen bonding potential, has some kinship with Ser, Thr, etc.

More substantial changes in functional or immunological properties are made by selecting substitutions that are less conservative, such as between, rather than within, the above five groups, which will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (a) substitution of gly and/or pro by another amino acid or deletion or insertion of Gly or Pro; (b) substitution of a hydrophilic residue, e.g., Ser or Thr, for (or by) a hydrophobic residue, e.g., Leu, Ile, Phe, Val or Ala; (c) substitution of a Cys residue for (or by) any other residue; (d) substitution of a residue having an electropositive side chain, e.g., Lys, Arg or His, for (or by) a residue having an electronegative charge, e.g., Glu or Asp; or (e) substitution of a residue having a bulky side chain, e.g., Phe, for (or by) a residue not having such a side chain, e.g., Gly.

The deletions and insertions, and substitutions according to the present invention are those which do not produce radical changes in the characteristics of the protein or peptide molecule. However, when it is difficult to predict the exact effect of the substitution, deletion, or insertion in advance of doing so, one skilled in the art will appreciate that the effect will be evaluated by routine screening assays, for example direct or competitive immunoassay of cytotoxicity or biological assay of T cell function as described herein. For non-superantigen homologues, the screening test(s) selected to assay function reflect the intrinsic functional activity of the native protein particularly its tumoricidal activity in the context of the inventions described herein. Modifications of such proteins or peptide properties as redox or thermal stability, hydrophobicity, susceptibility to proteolytic degradation or the tendency to aggregate with carriers or into multimers are assessed by methods well known to the ordinarily skilled artisan.

### Fusion Partners for Native SEs or SE Homologues

### Antibodies

In another embodiment, fusion protein partners for SAg or preferably SEG or SEG homologues include tumor specific antibodies, preferably F(ab′)₂, Fv or Fd fragments thereof, that are specific for antigens expressed on the tumor. In another embodiment, a fusion partner is a polypeptide ligand for a receptor expressed on tumor cells. These antibodies, fragments or receptor ligands may be in the form of synthetic polypeptides. The nucleic acid form of the antibody is envisioned which is useful as a fusion construct with the SAg DNA. Such a fusion protein is prepared using a fusion gene comprising nucleic acids encoding the SEG or SEG homologue and the tumor targeting structure. Methodology is described in the art and in Example 2. The vector for recombinant SEG production described herein is useful for this purpose. The same methodology can be used to fuse costimulatory molecules to SEG or an SEG homologue or any useful SAg or SAg homologue described herein.

One advantage of certain antibody constructs of the present fusion polypeptides is prolonged half-life and enhanced tissue penetration. Intact antibodies in which the Fc fragment of the Ig chain is present will exhibit slower blood clearance than their Fab′ fragment counterparts, but a fragment-based fusion polypeptide will generally exhibit better tissue penetrating capability.

Preferentially, the tumor targeting structure in the SEG, SEG homologue conjugate (e.g., tumor specific antibody, Fab or single chain Fv fragments or tumor receptor ligand) has a greater affinity for the tumor than the SAg in the conjugate has for the class II molecule thus preventing the SAg from binding all MHC class II receptors and favoring binding of the conjugate to the tumor. In the case of SEB, the dominant epitope for neutralizing antibodies 225-234 is recombinantly or biochemically bound to the tumor targeting molecule e.g., tumor specific antibodies, Fas or Fv fragments. In so doing, it sterically interferes with the recognition of the dominant epitope by preexisting antibodies.

To further enhance the affinity of the tumor specific antibody in the conjugate for tumor cells in vivo, tumor specific antibodies are used which are specific for more than one antigenic structure on the tumor, tumor stroma or tumor vasculature or any combination thereof. The tumor specific antibody or F(ab′)₂, Fab or single chain Fv fragments are mono or divalent like IgG, polyvalent for maximal affinity like IgM or chimeric with multiple tumor (tumor stroma or tumor vasculature) specificities. Thus, when the SE or SPE-MoAb conjugate is administered in vivo, it will preferentially bind to tumor cells rather than to endogenous SE antibodies or MHC class II receptors.

To reduce affinity of the SE-mAb conjugate for endogenous MHC class II binding sites, the high affinity Zn⁺⁺ dependent MHC class II binding sites in SEA, SEC2, SEC3, SED, SPEA, SPEC, SPEG, SPEH, SMEZ, SMEZ2, M. arthritides are deleted or replaced by inert sequence(s) or amino acid(s). These structural alterations in SE or SPEA reduce the affinity for MHC class II receptors from a K_{d} of 10⁻⁷ or 10⁻⁸ to 10⁻⁵. SEB, SEC and SSA and other SEs or SPEs do not have a high affinity Zn++ dependent MHC class II binding site but have multiple low affinity MHC class II binding sites (K_{d} 10⁻⁵-10⁻⁷). In these cases, alteration of the MHC class II binding sites is not always necessary to further reduce affinity for MHC class II receptors; at the very least mutation of one or two of the low affinity MHC class II binding sites will suffice in most instances.

Most importantly, tumor specific antibodies, Fab, F(ab′)₂ or single chain Fab or Fv fragments in the SAg-mAb conjugate have a higher affinity for tumor antigens (K_{d} 10⁻¹¹-10⁻¹⁴ or lower) than for the SAg has for MHC class II binding sites (K_{d} 10⁻⁵ to 10⁻⁷) and its dominant epitope has for SAg specific antibodies (K_{d} 10⁻⁷ to 10⁻¹¹). In this way, the conjugate will bind preferentially to the tumor target in vivo rather than preexisting antibodies or MHC class II receptors.

Antibody fragments are obtained using conventional proteolytic methods. Thus, a preferred procedure for preparation of F(ab′)₂ fragments from IgG of rabbit and human origin is limited proteolysis by the enzyme pepsin. Rates of digestion of an IgG molecule may vary according to isotype; conditions are chosen to avoid significant amounts of completely degraded IgG as is known in the art.

Fab fragments include the constant domain of the light chain (C_{L}) and the first constant domain (C_{H}) of the heavy chain. Fab′ fragments differ from Fab fragments by the addition of a few residues at the C-terminus of C_{H1} domain including one or more cysteine(s) from the antibody hinge region. F(ab′)₂ fragments were originally produced as pairs of Fab′ fragments that have hinge cysteines between them. Other chemical couplings of antibody fragments are also known.

An “Fv” fragment is the minimum antibody fragment that contains a complete antigen-recognition and binding site. This region consists of a dimer of one heavy chain and one light chain variable domain in tight, con-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “scFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables to allow pairing between the two domains on the same chain, the domains are forced to pair with the complementary domains of another chain and create two antigen-binding sites. Diabodies are described in EP 404,097 and WO 93/11161, incorporated herein by reference. “Linear antibodies”, which can be bispecific or monospecific, comprise a pair of tandem Fd segments (V_{H}-C_{H1}-V_{H}-C_{H1}) that form a pair of antigen binding regions.

An antibody fragment may be further modified to increase its half-life by any of a number of known techniques. Conjugation to non-protein polymers, such PEG and the like, is also contemplated

The antibody fusion partner for use in the present invention may be specific for tumor cells, tumor stroma or tumor vasculature. Antigens expressed on tumor cells that are suitable targets for mAb-SAg fusion protein therapy include erb/neu, MUC1, 5T4 and many others. Antibodies specific for tumor vasculature bind to a molecule expressed or localized or accessible at the cell surface of blood vessels, preferably the intratumoral blood vessels, of a vascularized tumor. Such molecules include endoglin (TEC-4 and TEC-11 antibodies), a TGFβ. receptor, E-selectin, P-selectin, VCAM-1, ICAM-1, PSMA, a VEGF/VPF receptor, an FGF receptor, a TIE, an αᵥβ₃ integrin, pleiotropin, endosialin and MHC class II proteins. Such antibodies may also bind to cytokine-inducible or coagulant-inducible products of intratumoral blood vessels. Certain preferred agents will bind to aminophospholipids, such as phosphatidylserine or phosphatidylethanolamine.

A tumor cell-targeting antibody, or an antigen-binding fragment thereof, may bind to an intracellular component that is released from a necrotic or dying tumor cell. Preferably such antibodies are mAbs or fragments thereof that bind to insoluble intracellular antigen(s) present in cells that may be induced to be permeable, or in cell ghosts of substantially all neoplastic and normal cells, but are not present or accessible on the exterior of normal living cells of a mammal.

Anti-tumor stroma antibodies bind to a connective tissue component, a basement membrane component or an activated platelet component; as exemplified by binding to fibrin, RIBS (receptor-induced binding site) or LIBS (ligand-induced binding site).

Fusion proteins include linkers or spacers that are incorporated into the recombinant fusion construct. Numerous types of disulfide-bond containing linkers are known that can be successfully employed to fuse the SAg to an antibody or fragment, certain linkers are preferred based on differing pharmacological characteristics and capabilities. For example, linkers that contain a disulfide bond that is sterically “hindered” are preferred, due to their greater stability in vivo, thus preventing release of the SAg moiety prior to binding at the site of action.

The SEG or SEG homologue-tumor targeting fusion proteins described above are administered parenterally, intratumorally, intrathecally (e.g., intraperitoneally, intrapleurally) by infusion or injection in conventional or sustained release vehicles in dosages of 0.01 ng/kg to 100 μg/kg every 3-7 days as comprehensively described herein in sections on Tumor Models and Examples 1 and 2.

Yersinia pseudotuberculosis is an Anti-Tumor Biologic

The present invention contemplates the use of Yersinia pseudotuberculosis (YTB) for treatment of cancer either as a vaccine against a prospective tumor or as a treatment for an established tumor in a mammal. The prior art indicates that YTB can preferentially target Tregs in a T3SS-dependent manner and directly modulates their stability and functional properties by decreasing Foxp3 and IL-10 expression, respectively, there are no reports that this organism or its variants can be useful in killing tumors cells in vivo and in vivo. Here we show that despite its virulence, YTB can be harnessed as an anti-tumor agent in vivo by diminishing Treg population in the tumor microenvironment and thereby skewing the intratumoral immune balance in favor of T effector cells capable of eliminating the tumor.

YTB can efficiently translocate effector molecules, so called Yersinia outer proteins (Yops), into target cells (Viboud G I, et al. Annu Rev Microbiol. 2005; 59:69-89). Yops initiate homeostatic localization of Ras homologous GTPase (Rho-GTPase) proteins, alter phosphorylation patterns, and activity of different small G-proteins, thereby deregulating actin cytoskeleton assembly and molecular activity. The cytotoxic necrotizing factor y (CNFY) exhibits its immunomodulatory effect by activating the Rho-GTPase proteins Rho A, Rac1, and Cdc42 (Schweer J, et al. PLoS Pathog. 2013; 9:e1003746). Rho-GTPase proteins are key components of T cell development, activation, differentiation, and migration, acting via modulation of the T cell cytoskeleton. Rap1, a Rho-GTPase protein, promotes thymic and peripheral Treg development (Li L et al. Cell Immunol. 2010; 266:7-13). Translocation of Yops via the type III secretion system (T3SS) requires direct cell-YTB contact enabled by invasins (inv). Invasin A acts as a potent 1 integrin ligand (mainly via α4β1 integrin) [Maldonado-Arocho F J et al. PLoS Pathog. 2013; 9:e1003415) enabling binding to β1 integrin-expressing cells such as epithelial cells and Foxp3+ Tregs.

YTB is a facultative anaerobic, gram-negative Enterobacteriaceae and is isolated from animals, soil and water. As a psychrophilic organism, YTB is able to grow at 4° C., and cold chain food products could offer a potential food safety hazard. YTB. strains belong to genetic groups 1 to 6 and serotypes 1a, 1b, 1c, 2a, 2b, 2c, 3, 4a, 4b, 5a, 5b, 6, 7, 8, 9, 10, 11, 12, 13, 14, and 15. The genetic group 3 (Far Eastern systemic-pathogenicity type)/serotypes 1b, 1c, 2a, 2b, 2c, 3, 4a, 4b, 5a, 5b, 6, 7, 8, 10, and 15 are the human pathogens in Japan, China, and Korea. The genetic group 2 (European gastroenteric-pathogenicity type)/serotypes 1a and 1b and genetic group 5/serotype 0:3 are the human pathogens in western countries. Although most strains of Y. pseudotuberculosis are melibiose positive, genetic group 4/serotypes 0:1, 0:5, 0:6, 0:7, 0:9, 0:10, 0:11, and 0:12 (nonpathogenic strains), which are distributed in the environment of Japan, and genetic group 5/serotype 0:3 are melibiose negative.

To facilitate attachment, invasion, and colonization of its host, YTB possesses many virulence factors. Superantigens, bacterial adhesions, and the actions of Yops (which are bacterial proteins once thought to be “Yersinia outer membrane proteins”) that are encoded on the “[plasmid] for Yersinia virulence”—commonly known as the pYV—cause host pathogenesis and allow the bacteria to live parasitically. Y. pseudotuberculosis can live extracellularly due to its formidable mechanisms of phagocytosis and opsonization resistance through the expression of Yops and the type III pathway but can invade host cells, especially macrophages, intracellularly to evade immune responses.

pYV

The 70-kb pYV is critical to Yersinia's pathogenicity, consisting of genes encoding virulence factors and its loss confers avirulence to all Yersinia species. A 26-kb “core region” in the pYV contains the ysc genes, which regulate the expression and secretion of Yops. Many Ysc proteins consolidate to form a type-III secretory apparatus, which secretes many Yops into the host cell cytoplasm with the assistance of the “translocation apparatus”, constructed of YopB and YopD. Cornelis G R et al. Microbiol. Mol. Biol. Rev. 62 (4): 1315-52 (1998)). The core region also includes yopN, yopB, yopD, tyeA, lcrG, and lcrV, which also regulate Yops gene expression and translocate secretory Yops to the target cell. YopN and TyeA act as a gate wherein only their conformational change on the cell surface will unblock the secretory pathway.

### Effector Yops

The Yops that act directly on host cells to cause cytopathologic effects. These “effector Yops” are encoded by pYV genes external to this core region (Lindler, L. (2004) Virulence plasmids of Yersinia: characteristics and comparison Funnell, B. E.; Phillips, G. J. (eds.). Plasmid biology. ASM Press. pp. 423-437). The combined function of these effector Yops permits the bacteria to resist internalization by immune and intestinal cells and to evade the bactericidal actions of neutrophils and macrophages. Inside the bacterium, these Yops are bound by pYV-encoded Sycs (specific Yop chaperones), which prevent premature interaction with other proteins and guide the Yops to a type-III secretory apparatus. LcrV, YopQ, YopE, YopT, YopH, YpkA, YopJ, YopM, and YadA are secreted by the type-III secretory pathway. LcrV inhibits neutrophil chemotaxis and cytokine production, allowing YTB to form large colonies without inducing toxicity and, with YopQ, contributes to the translocation of YopB and YopD to the cell membrane for pore-formation. By depolymerizing actin filaments, YopE, YopT, and YpkA resist endocytosis and phagocytosis by intestinal cells while retaining cytotoxicity for the host cell. YopT targets Rho GTPase and uncouples it from the membrane whereas YopE and YpkA GTPase activity converts Rho proteins to their inactive GDP-bound states. YpkA also catalyzes serine autophosphorylation and may undermine host cell immune response signal axes. YopH acts on host focal adhesion sites by dephosphorylating several phosphotyrosine residues on focal adhesion kinase (FAK) and the focal adhesion proteins paxillin and p130. Since FAK phosphorylation is involved in uptake of yersiniae as well as T cell and B cell responses to antigen-binding, YopH elicits antiphagocytic and other anti-immune effects. YopJ interferes with the mitogen-activated protein (MAP) kinase activities, p38, and extracellular signal-regulated kinase leading to macrophage apoptosis. In addition, YopJ inhibits TNF-α release through an inhibitory action on NF-κB. YopM is thought to limit host cell growth by binding to RSK (ribosomal S6 kinase), which regulates cell cycle regulation genes. The yop genes, yadA, ylpA, and the virC operon are considered the “Yop regulon”

### Adhesion

Y. pseudotuberculosis adheres strongly to intestinal cells via chromosomally encoded proteins[4] so that Yop secretion may occur, to avoid being removed by peristalsis, and to invade target host cells (Robins-Browne, R.; Hartland, E. (2003) “Yersinia species”. In Miliotis, Marianne D.; Bier, Jeffrey W. (eds.). International Handbook of Foodborne Pathogens. CRC Press. pp. 323-355). A transmembrane protein, invasin, facilitates these functions by binding to host cell αβ1 integrins (Miller V. Yersinia invasion genes and their products. ASM News. 58: 26-33 (1992)). Through this binding, the integrins cluster and activate FAK causing a reorganization of the cytoskeleton and internalization of bound bacteria. The protein encoded on the “attachment invasion locus” named Ail enables YTB attachment and invasion.

### Superantigens

Certain strains of YTB express a superantigenic exotoxin, YPM, or the Y. pseudotuberculosis-derived mitogen, from the chromosomal ypm gene. (Uchiyama T et al. J. Immunol. 151 (8): 4407-13(1993)). -YTB mitogens adopt a sandwich structure consisting of 9 strands in two beta sheets, in a jelly roll fold topology. YTB molecular weight is about 14 kDa. Structurally, it is unlike any other superantigen, but is remarkably similar to the tumor necrosis factor and viral capsid proteins. YPM specifically binds and causes the proliferation of T lymphocytes expressing the V03, V07, V08, V09, V013.1, and V13.2 variable regions (Carnoy C et al. Adv. Exp. Med. Biol. 529; 133-5 (2003)) with CD4+ T cell preference, although activation of some CD8+ T cells occurs. Since administering anti-TNF-α and anti-IFN-γ monoclonal antibodies neutralizes YPM toxicity in vivo, these cytokines are largely responsible for the damage caused indirectly by the exotoxin. Strains that carry the exotoxin gene are rare in Western countries, where the disease produces minor symptoms, whereas more than 95% of strains from Far Eastern countries contain ypm and are correlated with Izumi fever and Kaasaki disease. Although the superantigen poses the greatest threat to host health, all virulence factors contribute to YTB. pseudotuberculosis viability in vivo and define the bacterium's pathogenic characteristics.

Yersinia pseudotuberculosis Mitogen (Superantigen) (YPM)

Cloning, expression and purification of YPM is described by Miyoshi-Akiyama, T. et al., J. Immunol. 154: 5228-5234 (1995). The sequence of YPM is shown below (Carnoy, C. et al., J. Bacteriol. 184 (16), 4489-4499 (2002))

SEQ ID NO: 9

| | |
|---|---|
| 1 | MKKKFLSLLT LTFFSGLALA ADYDNTLNSI PSLRIPNIET |
| | YTGTIQGKGE VCIRGNKEGK |
| 61 | SRGGELYAVL RSTNANADMT LILLCSIRDG WKEVKRSDID |
| | RPLRYEDYYT PGALSWIWEI |
| 121 | KNNSSEASDY SLSATVHDDK EDSDVLMKCP |

### Isolation of YTB Using Selective Agar Media

Pure strains of YTB are prepared on blood agar or other nutrient agar. The strains are oxidase negative and Gram stain negative. Isolation of YTB is carried out using Cefsulodin-Irgasan-Novobiocin agar (CIN agar, Difco, Oxoid) and CIN agar containing 0.1% esculin and 0.05% ferric citrate (modified virulent Yersinia enterocolitica agar (mVYE agar)) and incubated at 30° C. for 24 h. YTB forms dark pin colonies as a result of esculin hydrolysis and is easily differentiated from most nonpathogenic Yersinia organisms by the characteristic deep red center (“bull's eye”) with a transparent margin and diameter 2-4 mm appearance of Yersinia colonies. Serotyping of YTB is carried out by slide agglutination commercial antisera O:3, O:5, O:8, and O:9 or by O-genotyping using O-antigen gene cluster-specific PCRs.

The pyrazinamidase test is useful for the chromosomal phenotypic identification to distinguish pathogenic from nonpathogenic strains of YTB. Pathogenic YTB show negative reactions while nonpathogenic YTB strains are positive turning brownish pink in the presence of ferrous salts.

Using PCR, pathogenic YTB can be detected in samples rapidly and with high specificity and sensitivity. Several PCR assays have been developed to detect pYV-positive Y. enterocolitica and Y. pseudotuberculosis in clinical, food, and environmental samples. Many of these samples use primers targeting the yadA or virF gene located on pYV. The inv gene, located in the chromosome of YTB strains is the most frequently used target. Multiplex PCR method using a mixture of primers against inv (5′-TAAGGGTACTATCGCGGCGGA-3′ (SEQ ID NO: 10 and 5′-CGTGAAATTAACCGTCACACT-3′) SEQ ID NO 11, ail (5′-ACTCGATGATAACTGGGG AG-3′ (SEQ ID NO:12) and 5′-CCCCCAGTAATCCATAAAGG-3′) SEQ ID NO: 13), and virF (5′-TCATGGCAG AACAGCAGTCAG-3′ (SEQ ID NO: 14) and 5′-ACTCAT CTTACCATTAAGAAG-3′) (SFQ ID NO:15) has been designed to detect YTB.

Real-time PCR assays based on “Tagman” and “SYBR Green” approaches are useful. The Tagman system is a 5′-nuclease assay that utilizes specific hybridization of a dual-labelled Taqman probe to the PCR product. The SYBR Green system is based on the binding of the fluorescent SYBR Green dye to the PCR product. Chromosomally encoded ail and yst genes, the plasmid-bome yadA gene and a Yersinia-specific region of the 16S rRNA gene have been used in real-time PCR. Pathogenic YTB. strains yield positive PCR products from the yadA gene. Using SYBRGreen real-time PCR assay, the Tm values of this yadA primer pair (yadA-F1757: 5′-ACGAGTTGACAAAGGTTTAGCC-3′ (SEQ ID NO: 16) and yadA-R1885: 5′-GAACCAACCGCTAATGCCTGA-3′) (SEQ ID NO:17) are different between the pathogenic Y. enterocolitica (82.2° C.) and Y. pseudotuberculosis (81.5° C.) strains. Therefore, this primer pair is useful for detection and differentiation of the two pathogenic Yersinia species.

### YTB Produces an Anti-Tumor Response

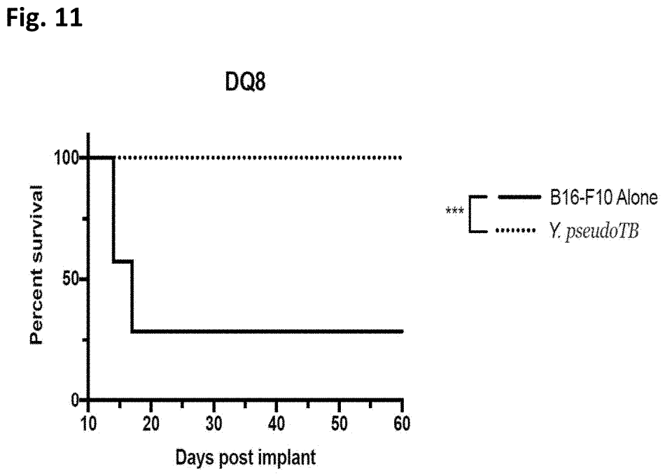

The ability of YTB to prevent the outgrowth of B16F10 melanoma in HLA-DQ8 mice was examined in the intraperitoneal tumor model. B16F10 cells (2.5×10⁵) were injected intraperitoneally in groups of 5 mice. The experimental group received live YTB in doses of 10⁴ ip on days −2 and +4. Live B16F10 cells were delivered ip on day 0. Control group was injected ip with 2.5×10⁵ B16F10 tumor cells alone but received no treatment. Tumor outgrowth and survival was analyzed. Results showed that YTB treatment in extended survival for more than 60 days whereas untreated tumor bearing controls succumbed to their tumor by day 15. Log-rank (Mantel-Cox) (p>0.01) (FIG. 11).

Murine carcinoma and lymphoma models disclosed in U.S. Pat. No. 8,128,931 incorporated in entirety by reference are useful to demonstrate the anti-tumor effect of YTB. The methodologic principles deployed in these models are similar. Tumor cells are injected and allowed to grow to dimensions of at least 0.5 cm in two diameters. YTB is injected parenterally in doses ranging from 10⁴-10⁶ live cells. The dosage may be repeated weekly for up to 6 weeks. The YTB is administered intratumorally, intralymphatically into tumor draining lymph nodes, subcutaneously, intradermally, intraperitoneally, intrapleurally, intravenously or intra-arterially. Tumor are measured daily with calipers and scored using the equation: tumor size (mm³)=length×(width) 2/2. Control mice are similarly injected with tumors, but treatment is withheld. Statistical methods comparing measurements of treated versus control tumors deploy two tailed Students T test and/or Anova with Bonferonni post-test.

### Ramifications

As the most powerful T cell agonists known we revisit the bacterial superantigens (SAg) in an effort to exploit and harness their hidden potential for cancer treatment. In this comprehensive analysis we introduce evolutionarily modified SAgs that work together with endogenous HLA-DQ8 alleles to produce a sustained anti-tumor response and long-term melanoma survival devoid of toxicity. Central to the anti-tumor effect are SEG-SEI staphylococcal enterotoxins (SEs) that display the unique ability to induce potent T cell stimulation together with muted cytokine activation. This dichotomous response produced a powerful acute and long-term tumoricidal response devoid of TNFα/IL-6-mediated toxicity. For translational relevance, we used humanized MHCII mice expressing the DQ8αβ (DQA*03:01, DQB*03:02) allele. These mice provide an accurate representation of human-like responses to SAgs while the HLA-DQ8 allele possesses a large polymorphic antigen binding groove capable of engaging melanoma neoantigens. In this model, SEG-SEI primed, expanded, mobilized, and recruited a T effector cell population to the TME along with T cell chemoattractants and tumoricidal cytokines. Remarkably, long-term surviving mice exhibited specific anti-melanoma memory, rejecting live melanoma but not the unrelated LLC. Histopathology corroborated the major anti-tumor response. These findings illuminate for the first time a heretofore unrecognized SAg-MHCII complementation that unleashes a powerful anti-tumor chain of events culminating in long-term melanoma survival.

CD8+ T effector cells produced in response to SEG-SEI in HLA-DQ8 tg mice appear to be the major mediators of the anti-tumor response. This notion is supported by the increased levels of T effector cells in the TME and the tumor injection site following SEG-SEI treatment in HLA-DQ8 tg mice not seen in untreated controls. These T effector cells appeared to be melanoma specific since in both vaccination and established tumor models SEG-SEI-treated HLA-DQ8 tg mice rejected rechallenge with parental melanoma but were unable to eradicate the unrelated Lewis lung carcinoma (FIGS. 2B,C, 6A,B). The T-cell memory response was also long-lived since mice were protected against melanoma rechallenge delivered 160 days after initial primary tumor challenge. The effectiveness of T effector/memory cells in tumor killing was likely enhanced by the minimal presence of Tregs and myeloid cells in the TME of SEG-SEI-treated HLA-DQ8 tg mice (FIGS. 3A-D). Indeed, the consequent intratumoral Teffector/Treg imbalance likely tilted the immune balance in the TME in favor of tumor deletion. HLA-DQ8 in concert with SEG-SEI appear to be critical in the tumoricidal effect and minimizing toxicity. This notion is supported by the absent antitumor effect and appearance of toxicity in HLA-DR3 tg mice following a second dose of SEG-SEI (FIG. 4A). Likewise, SEG-SEI appears to be requisite since other SAgs such as SEB and SpeA in doses 5-10-fold lower than SEG-SEI induced severe toxicity in HLA-DQ8 tg mice (Roy C J et al. Infect. Immun. 73,2452-2460 (2005); Rajagopalan G et al. Infect Immun. 72,6733-6737 (2004); Yeung R S M et al. Eur J Immunol 26,1074-1082 (1996); DaSilva L et al. J Infect Dis. 185,1754-60 (2002)). The differential toxicity of SEG-SEI in HLA-DR3 tg and HLA-DQ8 tg mice be explained by the massive increase in TNFα, IFNγ and IL-6 levels in HLA-DR3 tg mice that was not seen in HLA-DQ8 tg mice (FIGS. 4A,B,C) Diverse cytokine reactions and toxicity of this order in humanized MHCII transgenic mice have been noted in response to SAgs and ascribed to their divergent affinities and binding conformations with human MHCII molecules (Mangalam A K, J Immunol. 190, 513-518 (2013); Li H, Annu Rev Immunol. 17:435-461 (1999)). Such differential cytokine responses also appear to be dependent on interactions of conserved SAg motifs with the B7-CD28 axis (Arad G et al. supra (2011); Levy R et al. supra (2016)). Indeed, cytokine-based toxicity of SAgs was abrogated in humanized MHCII mice deficient in CD28 or by treatment with anti-CTLA-Fc antibody (Rajagopalan G et al Int Immunol. 14, 801-12 (2002)); Rajagopalan G et al. Infect Immun. 72,6733-6737 (2004). Taken together, these findings indicate that SEG-SEI works selectively with HLA-DQ8 allele and possibly the B7-CD28 axis to preserve melanoma specific anti-tumor response while minimizing cytokine-based toxicity.

MHCII molecules appear to have an increasingly important role in tumor neoantigen presentation and activation of CD4+ T cells for priming of tumor specific CD8+T effector cells and collaborating with them in an effective anti-tumor response at the tumor site (Borst, J., et al. Nat. Rev. Immunol. 18, 635-647 (2018); Alspach E et al. Nature. 574,696-701 (2019). These notions accord with our deletion experiments showing that both CD4+ and CD8+ T cell are required for the SEG-SEI-mediated induced anti-tumor response (FIG. 2G). Recent reports have further linked therapeutic outcome in response to immunotherapy to the number of mutations or putative neoantigens derived from somatic mutations in multiple malignancies (Le, D. T. et al. Mismatch-repair deficiency predicts response of solid tumors to PD-1 blockade. Science 357, 409-413 (2017); Luksza M et al. Nature. 551. 517-520 (2017)). Indeed, our findings suggest a critical role of the tumor release of tumor cell antigens (tumor neoantigens or tumor associated antigens) and SEG-SEI for an effective anti-tumor response. In support of this notion, effective protection from live melanoma challenge required (i) the contribution of both tumor cell inoculation and SEG-SEI treatment and (ii) that tumor inoculation precede SEG-SEI delivery (FIGS. 2B,6A). Thus, following melanoma inoculation, melanoma antigens are processed by antigen presenting cells with consequent priming of both CD4+ and CD8+ T cells. It is highly likely that HLA-DQ8 plays a key role at this early stage of melanoma antigen processing and presentation. Indeed, melanoma neopeptides have shown preferential affinity for HLA-DQ8 allele and B16-F10 melanoma neopeptides with high affinity for MHCII have been shown to confer protection against tumor challenge when delivered on cationic liposomes (Linnemann C et al. Nat Med. 21, 81-85 (2015); Kreiter S et al. Nature 520, 692-696 (2015)).

On day 6 and 9 after tumor inoculation SEG-SEI administration resulted in expansion of these antigen-primed CD4+ and CD8+ T-cells. The expanded tumor reactive CD4+ T-cell population likely further enhanced melanoma antigen priming while also promoting CD8+ T-cell differentiation into T effector cells (Fehlings M et al. Nat Commun 2017; 8:562; Wei S C et al. Cell 170:1120-33 (2017). The very same superantigenic stimulus generated T cell chemotactic molecules (FIGS. 5C,D) that enabled recruitment of CD8+T effector cells to the site of tumor injection and the TME (FIGS. 3A,B). SEG-SEI also stimulated a surge of IFNγ output from both CD4+ and CD8+ T-cells (FIG. 1C,D,4A). By days 13-16 after tumor implant, the TME was infiltrated by CD8+T effector cells along with IFNγ and TNFα that appear to constitute the major tumoricidal effectors (FIGS. 3A,B,4I). The acute tumoricidal response (FIG. 5G,H) was therefore likely mediated by clones of primed melanoma-specific effector T-cells while a memory cell population derived from this lineage provided tumor specific protection against late melanoma challenge at day 160 (FIG. 2C). Collectively, these findings highlight the central role for HLA-DQ8 allele and SEG-SEI in generating a neoantigen-primed tumor reactive T-cell population that drives an acute tumoricidal response as well as long-term T-cell memory. This latter feature constitutes a critical distinction from T cell agonist anti-PD-1 whose long-term T-cell memory response is epigenetically constrained (86). The ability of the (SEG-SEI)-HLA-DQ8 system to generate both acute and long-term anti-tumor effects thereby appears to constitute a novel immunotherapeutic advance,

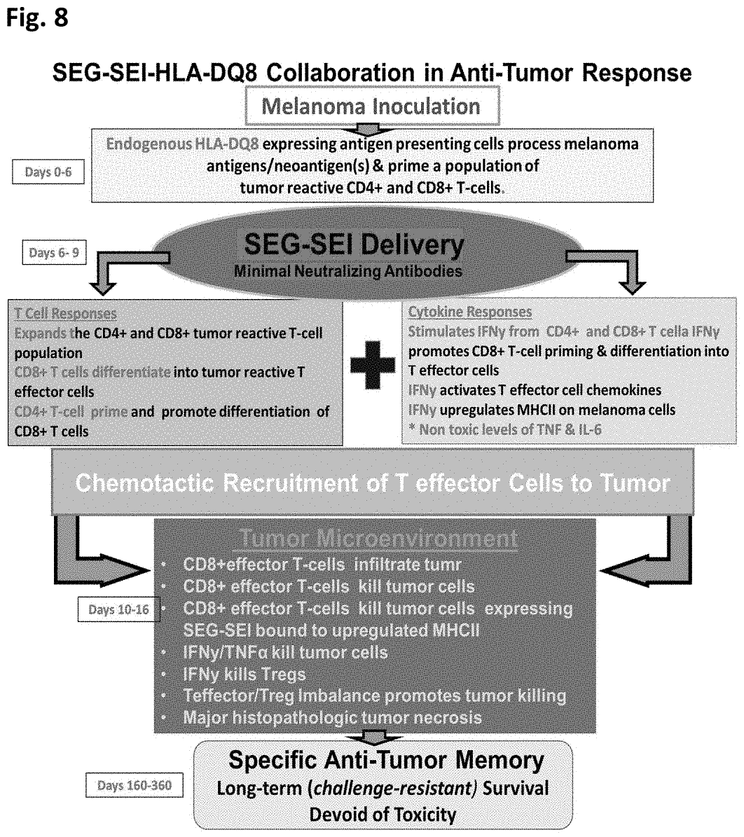

IFNγ produced by CD4+ and CD8+ T cells in response to SEG-SEI emerges in a major role as a tumoricidal effector of its own while also priming tumor reactive CD8+ T cells. Following SEG-SEI injection on day 6 SEG-SEI-treated mice displayed robust IFNγ production (FIG. 4A). This likely upregulated MHCII expression on melanoma cells enabling them to bind SEG-SEI and activate CD8+T effector cells (Dohlsten M et al. Cancer Immunol. Immunother. 41,162-168 (1995)). The same IFNγ surge was likely responsible for constraining Treg differentiation to account for minimal levels of Tregs noted in the TME after SEG-SEI treatment (FIGS. 1G, 3D) (Chang J H et al. Eur J Immunol) 39,1241-1251 (2009). Such Treg reduction in the TME would also likely support the CD8+ effector T cell response. Melanoma specimens from subjects with improved prognosis exhibited an assemblage of chemokines that were strikingly similar to the chemokine profile we detected in tumors after SEG-SEI treatment (FIG. 4J). Several of these have been shown to be IFNγ and SAg inducible and are known to recruit effector T cells akin to those we noted in the TME of SEG-SEI-treated mice (FIGS. 5A-F). Interestingly, we also found TNFα together with IFNγ in the TME of SEG-SEI-treated HLA-DQ8 tg mice (FIG. 4I). The unique persistence in time of high affinity, zinc-binding SAgs such as SEI on the surface of antigen-presenting cells may also enable a self-perpetuating cycle that generates T effector cells, IFNγ and T cell memory (Pless D D et al. Infect Immun. 73, 5358-5366 (2005); Femndez M M et al. Mol Immunol. 43, 927-938 (2006)). Collectively, the combined presence of IFNγ and TNFα together with T effector cells in the TME strongly supports their importance as key contributors to a dynamic tumoricidal process that produces long-term memory and tumor challenge-resistant melanoma survival. A schematic representation of the proposed chain of events is shown in FIG. 8.

In addition to treatment of established tumors, the long-lived protection devoid of toxicity and tumor specific protection exhibited by SEG-SEI in our vaccination studies have clear relevance for vaccine design in susceptible human populations. The tumor specificity afforded by vaccination with irradiated melanoma cells was evident from the resistance these mice exhibited to melanoma but not to unrelated LLC challenge in vaccinated mice. Vaccinated mice that resisted a second melanoma challenge showed >90% reduction of tumor density in the omentum relative to unvaccinated controls. In these mice, the limited number of melanoma cells in the omentum were surrounded by large aggregates of mononuclear cells, strikingly devoid of stromal organization and discrete nodule formation seen in the untreated control tumors. T cells harvested from the peritoneal lavage showed a mixture of T cells with activating and memory phenotypes not seen in the untreated control suggesting that a T cell recall response to melanoma was responsible for the anti-tumor effect. Collectively these findings suggest that vaccination enabled a memory T cell-mediated, melanoma-specific response to tumor rechallenge, constrained the number of melanoma cells in the omentum and limited the evolution of a mature tumor network.

Significant constitutional and hemodynamic toxicity has precluded use of canonical wild type SEA and SEB for human cancer treatment (Alpaugh R K et al. Clin Cancer Res 4, 1903-1914 (1998); Terman D S Crit Rev Oncol Hematol. 4:103-24 (1985)). Genetically modified SEA fused to a tumor specific antibody showed reduced toxicity in humans but limited clinical efficacy largely due to the presence of pre-existing sero-reactive neutralizing antibodies in up to 80% of patients. Notably, a small cohort of subjects with long-term responses to SEA therapy displayed low to undetectable levels of such neutralizing antibodies suggesting that the SAg could be effective in their absence (31). The low incidence of neutralizing antibodies against SEG and SEI in human sera and their negligible toxicity when combined with HLA-DQ8 suggests that this complex may enable a larger percentage of cancer patients to access of SAg treatment.

For clinical translation, the broad distribution of HLA-DQ8 suggests that SEG-SEI may be work effectively by direct administration to patients (Maiersa M, Human Immunology 68, 779-788 (2007). A clue suggesting that this route might be well tolerated emerges from the lack of toxicity and survival benefit induced by a staphylococcal aureus filtrate preparation containing egcSEs (including SEG and SEI) used in a pilot study of 14 patients with advanced lung cancer and pleural effusion (Terman D S et al. Clin Chest Med. 27: 321-34 (2006)). Alternatively, to avert any potential toxicity mediated by the interaction of SEG-SEI with host alleles such as HLA-DR3, SEG-SEI could also be loaded onto HLA-DQ8 expressing APCs or inert particles to induce a population of melanoma specific T effector cells. HLA-DQ8 expressing dendritic cells, macrophages, derivatized erythrocytes or nanoparticles could serve as vehicles for SEG-SEI affixed to their surfaces. Small amounts of SAgs presented on dendritic cells and macrophages have been shown to initiate robust T cell responses in wild type mice (Bhardwaj N et al. J. Exp. Med. 178, 633-642 (1993); Ganem M B et al. PLoS ONE (2013) 8(6): 2013. e66244. doi:10.1371/journal.pone.006624).

In summary, we introduce evolutionarily modified SEG-SEI devoid of neutralizing antibodies that work in concert with the HLA-DQ8 allele hitherto unrecognized for its therapeutic properties. This combination enriched and mobilized a neoantigen-primed T effector cell population that propagated an acute tumoricidal response and long-term T-cell memory/survival. The selection of these agents and revelation of their dynamic anti-tumor collaboration with tumor antigens/neoantigens in humanized mice constitutes a major conceptual advance with compelling translational potential.

### Pharmaceutical Compositions and Administration

One or more homologues and fusion proteins are administered by parenterally, intravenously, intra-arterially by injection, infusion or instillation or intratumorally, intradermally or subcutaneously. SEG-SEI agents are most commonly administered intrapleurally or intraperitoneally in patients with malignant tumors. For example, malignant pleural or peritoneal disease with patients with lung, gastric, colon, breast, or ovarian cancer. SEG-SEI compositions, homologues and fusion proteins may also be administered to patients without malignant involvement or fluid accumulation in the cavitary space or its membranes but with primary or metastatic tumor of the organ (e.g., lung, stomach) and/or lymph nodes. For example, SEG-SEI compositions, homologues and fusion proteins may be administered intrapleurally or intraperitoneally to patients with primary lung cancer or lung metastases from other primary tumors (e.g., breast, ovary, gastric) without malignant involvement of the pleura or pleural space. In each of the above examples, SEG and SEI compositions, homologues and fusion proteins may be administered simultaneously or sequentially.

SEG-SEI compositions, homologues and fusion proteins are administered every 3-10 days for up to 30 three months. Dosages of SEG and SEI SEG-SEI compositions, homologues and fusion proteins range from 0.1 pg-1 ng/kg for each one. SEG-SEI agents are also administered intratumorally to stimulate a T cell-based inflammatory response, including release of tumoricidal cytokines and induction of cytotoxic T cells. The dosages of SEG-SEI compositions, homologues and fusion proteins administered to a single tumor site ranges from about 0.05-1 ng/kg body weight.

The pharmaceutical compositions of the present invention will generally comprise an effective amount of at least a SEG-SEI composition, homologues or fusion proteins dissolved or dispersed in a pharmaceutically acceptable carrier or aqueous medium. Combined therapeutics are also contemplated, and the same type of underlying pharmaceutical compositions may be employed for both single and combined medicaments. The intratumoral composition can be administered by needle or catheter via percutaneous entry or via endoscopy, bronchoscopy, culdoscopy or other modes of direct vision including directly at the time of surgery. The composition can be localized into the tumor with CT and/or ultrasound guidance.

With each drug in each tumor, experience will provide an optimum level. One or more administrations may be employed, depending upon the lifetime of the drug at the tumor site and the response of the tumor to the drug. Administration may be by syringe, catheter or other convenient means allowing for introduction of a flowable composition into the tumor. Administration may be every three days, weekly, or less frequent, such as biweekly or at monthly intervals.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by U. S. Food and Drug Administration. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

### Injectable Formulations

The SEG-SEI compositions, homologues and fusion proteins of the present invention are preferably formulated for parenteral administration, e.g., introduction by injection, infusion or instillation directly into an affected organ cavity or tumor (intratumorally). Means for preparing aqueous compositions that contain the SAg compositions are known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions and sterile powders for the extemporaneous preparation of sterile injectable solutions or dispersions. In all cases, the form should be sterile and fluid to the extent that syringability exists. It should be stable under the conditions of manufacture and storage and should be preserved against the contaminating action of microorganisms, such as bacteria and fungi.

The SEG and SEI compositions, homologues and fusion proteins can be formulated into a sterile aqueous composition in a neutral or salt form. Solutions as free base or pharmacologically acceptable salts can be prepared in water. Pharmaceutically acceptable salts, include the acid addition salts (formed with the free amino groups of the protein), and those that are formed with inorganic acids such as, for example, hydrochloric or phosphoric acids, or such organic acids as acetic, trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Suitable carriers include solvents and dispersion media containing, for example, water. In many cases, it will be preferable to include isotonic agents, for example, sugars or sodium chloride. Sterile injectable solutions are prepared by incorporating the active agents in the required amount in the appropriate solvent with various of the other ingredients enumerated above, as desired, followed by filtered sterilization. Suitable pharmaceutical compositions in accordance with the invention will generally include an amount of the SAg composition admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, or most recent edition, incorporated herein by reference. Endotoxin contamination should be kept minimally at a safe level, for example, less than 0.5 ng/mg protein. Moreover, for human administration, preparations should meet sterility, pyrogenicity, general safety and purity standards as required by the U.S. Food and Drug Administration. Upon formulation, the therapeutic compositions are administered in a manner compatible with the dosage formulation and in such amount as is therapeutically effective.

Tumors that can be Treated by SEG-SEI Compositions, Homologues, Fusion Proteins and Yersinia Pseudotuberculosis

The compositions of the claimed invention are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; 5 urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges 10 including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

### Example 1

### Clinical Trial of SEG-SEI Compositions

All patients treated have histologically confirmed malignant disease including carcinomas, sarcomas, melanomas, gliomas, neuroblastomas, lymphomas and leukemia and have failed conventional therapy. All patients' sera are tested for neutralizing antibodies against individual SEG and SEI using the inhibition of the T proliferation and primary binding neutralizing antibody assays All patients show baseline binding levels of the SEG and SEI and minimal inhibition of SEG-SEI induced T cell proliferation. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4(widespread metastases). Patient history is obtained, and physical examination performed along with conventional tests of cardiovascular and pulmonary function and appropriate radiologic procedures. They have not been undergoing any other anticancer treatment for at least one month and have a clinical Karnofsky performance status of at least 50. Histopathology is obtained to verify malignant disease.

The SEG and SEI tested include wild type SEs, fragments, homologues or fusion proteins described herein. SEG and SEI are administered intravenously, intraperitoneally, intrapleurally or intratumorally by infusion, injection, instillation or implantation in doses of 0.01 pg-100 ng. It may be administered every 2-7 days for up to 10 doses. It may be continued daily for up to 3 days after each infusion. The treatments are generally given every 2-7 days for a total of 1-12 treatments. However, the treatment schedule is flexible and may be given for a longer of shorter duration depending upon the patients' response.

Patient Evaluation:

Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al., supra)

The efficacy of the therapy in a patient population is evaluated using conventional statistical methods, including, for example, the Chi Square test or Fisher's exact test. Long-term changes in and short-term changes in measurements are evaluated separately.

| | | | Patients |
|---|---|---|---|
| Patients/Tumors | No. | Response | Responding (%) |
| All patients | 1231 | CR + PR | 83.5 |
| Tumor Type | No. | Response | Response (%) |
| Breast adenocarcinoma | 120 | CR + PR + <PR | 89 |
| Gastrointestinal carcinoma | 100 | CR + PR + <PR | 81 |
| Lung Carcinoma | 130 | CR + PR + <PR | 92 |
| Brain glioma/astrocytoma | 75 | CR + PR + <PR | 85 |
| Prostate Carcinoma | 100 | CR + PR + <PR | 87 |
| Lymphoma/Leukemia | 80 | CR + PR + <PR | 72 |
| Head and Neck Cancer | 80 | CR + PR + <PR | 76 |
| Renal and Bladder Cancer | 110 | CR + PR + <PR | 93 |
| Melanoma | 90 | CR + PR + <PR | 84 |
| Neuroblastoma | 80 | CR + PR + <PR | 80 |
| Prostate carcinoma | 100 | CR + PR + <PR | 85 |
| Uterine/Cervical | 100 | CR + PR + <PR | 78 |

| RESPONSE | DEFINITION |
|---|---|
| Complete remission | Disappearance of all evidence of disease |
| (CR) | |
| Partial remission (PR) | □50% decrease in the product of |
| | the two greatest perpendicular |
| | tumor diameters; no new lesions |
| Less than partial | 25%-50% decrease in tumor size, |
| remission (<PR) | stable for at least 1 month |
| Stable disease | <25% reduction in tumor size; |
| | no progression or new lesions |
| Progression | ≥25% increase in size of any one |
| | measured lesion or |
| | appearance of new lesions despite |
| | stabilization or remission of |
| | disease in other measured sites |

### Results

A total of 1231 patients are patients treated. The number of patients for each tumor type and the results of treatment are summarized in Table 1. Positive tumor responses are observed in as high as 75-90%% of the patients with breast, gastrointestinal, lung, prostate, renal and bladder tumors as well as melanoma and neuroblastoma.

Patients with all tumors exhibit objective clinical responses for an overall response rate of 82%. Tumors generally start to diminish, and objective remissions are evident after four weeks of combined SAgs and chemotherapy. Responses endure for an average of 24 months.

### Table 1

Toxicity consists of mild short-lived fever, fatigue and anorexia not requiring treatment.

The incidence of side effects (as % of total treatments) are as follows: chills—10; fever—10; pain—5; nausea—5; respiratory—3; headache—3; tachycardia—2; vomiting—2; hypertension—2; hypotension—2; joint pain—2; rash—2; flushing—1; diarrhea—1; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly after treatments.

### Example 2

### Clinical Trial of SEG Homologue SEG-R47

All patients treated have histologically confirmed malignant disease including carcinomas, sarcomas, melanomas, gliomas, neuroblastomas, lymphomas and leukemia and have failed conventional therapy. In cohorts 1 and 3, patient sera shows SEG binding levels of <95 ng/ml (minimal binding). Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4(widespread metastases). Patient history is obtained, and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

SEG-R47 is administered by intravenous infusion in doses of 0.01 pg-100 ng. It is administered every 2-7 days for up to 12 doses. However, the treatment schedule is flexible and may be given for a longer of shorter duration depending upon the patients' response.

Patient Evaluation: Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al., supra)

The efficacy of the therapy in a patient population is evaluated using conventional statistical methods, including, for example, the Chi Square test or Fisher's exact test. Long-term changes in and short-term changes in measurements are evaluated separately.

| RESPONSE | DEFINITION |
|---|---|
| Complete remission | Disappearance of all evidence of disease |
| (CR) | |
| Partial remission (PR) | >50% decrease in the product of the two |
| | greatest perpendicular |
| | tumor diameters; no new lesions |
| Less than partial | 25%-50% decrease in tumor size, stable |
| remission (<PR) | for at least 1 month |
| Stable disease | <25% reduction in tumor size; |
| | no progression or new lesions |
| Progression | ≥25% increase in size of any one |
| | measured lesion or |
| | appearance of new lesions despite |
| | stabilization or remission of |
| | disease in other measured sites |

### Results

A total of 1011 patients with serum SEG binding levels <95 ng/ml are treated with SEG-R47. The number of patients for each tumor type and the results of treatment are summarized in Table 2

TABLE 2

| | | | All Patients |
|---|---|---|---|
| Patients/Tumors | No. | Response | Responding (%) |
| All patients | 1011 | CR + PR | 81.7 |
| Tumor Type | No. | Response | Response (%) |
| Breast adenocarcinoma | 100 | CR + PR + <PR | 87 |
| Gastrointestinal carcinoma | 100 | CR + PR + <PR | 89 |
| Lung Carcinoma | 140 | CR + PR + <PR | 96 |
| Brain glioma/astrocytoma | 60 | CR + PR + <PR | 82 |
| Prostate Carcinoma | 100 | CR + PR + <PR | 81 |
| Lymphoma/Leukemia | 80 | CR + PR + <PR | 73 |
| Head and Neck Cancer | 80 | CR + PR + <PR | 75 |
| Renal and Bladder Cancer | 70 | CR + PR + <PR | 92 |
| Melanoma | 50 | CR + PR + <PR | 85 |
| Neuroblastoma | 50 | CR + PR + <PR | 89 |
| Prostate carcinoma | 100 | CR + PR + <PR | 88 |
| Uterine/Cervical | 100 | CR + PR + <PR | 72 |

Toxicity consists of mild short-lived fever, fatigue and anorexia not requiring treatment.

The incidence of side effects (as % of total treatments) are as follows: chills—10; fever—10; pain—5; nausea—5; respiratory—3; headache—3; tachycardia—2; vomiting—2; hypertension—2; hypotension—2; joint pain—2; rash—2; flushing—1; diarrhea—1; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. CBC, renal and liver functions tests do not change significantly after treatments.

### Example 3

### Clinical Trial of SEG Fusion Protein

All patients treated have histologically confirmed malignant disease including carcinomas, sarcomas, melanomas, gliomas, neuroblastomas, lymphomas and leukemia and have failed conventional therapy.

Patients diagnosed at any stage of metastatic disease are eligible. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained, and physical examination performed along with conventional tests of cardiovascular and pulmonary function and appropriate radiologic procedures. They have not been undergoing any other anticancer treatment for at least one month and have a clinical KPS of at least 50. Histopathology is obtained to verify malignant disease.

Fusion protein SEG-FP (SEG fused to a tumor specific antibody, Fab of fv fragment or tumor associated receptor) is administered to cohorts 1 and 2. The SEG-FP is administered by intravenous infusion, in doses of 0.01 pg-100 ng every 2-7 days for up to 12 doses. However, the treatment schedule is flexible and may be given for a longer of shorter duration depending upon the patients' response.

Patient Evaluation: Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al., supra)

The efficacy of the therapy in a patient population is evaluated using conventional statistical methods, including, for example, the Chi Square test or Fisher's exact test. Long-term changes in and short-term changes in measurements are evaluated separately.

| RESPONSE | DEFINITION |
|---|---|
| Complete remission | Disappearance of all evidence of disease |
| (CR) | |
| Partial remission (PR) | >50% decrease in the product of the |
| | two greatest perpendicular |
| | tumor diameters; no new lesions |
| Less than partial | 25%-50% decrease in tumor size, |
| remission (<PR) | stable for at least 1 month |
| Stable disease | <25% reduction in tumor size; |
| | no progression or new lesions |
| Progression | ≥25% increase in size of any one |
| | measured lesion or |
| | appearance of new lesions despite |
| | stabilization or remission of |
| | disease in other measured sites |

### Results

A total of 987 patients with serum SEG binding levels <95 ng/ml are treated. The number of patients for each tumor type and the results of treatment are summarized in Table 13. Objective tumor responses are observed in 75-90%% of the patients with breast, gastrointestinal, lung, prostate, renal and bladder tumors as well as melanoma and neuroblastoma. Tumors generally start to diminish, and objective remissions are evident after eight weeks of SEG treatment. Responses endure for an average of 24 months.

A total of 1048 patients with SEG binding levels >95 ng/ml are also treated. Results summarized in Table 3 show that no objective tumor remissions and tumor progression in all.

TABLE 3

| | | | Patients |
|---|---|---|---|
| Patients/Tumors | No. | Response | Responding (%) |
| All patients | 1156 | CR | 79.1 |
| Tumor Type | No. | Response | Response (%) |
| Breast adenocarcinoma | 100 | CR + PR + <PR | 86 |
| Gastrointestinal carcinoma | 100 | CR + PR + <PR | 81 |
| Lung Carcinoma | 150 | CR + PR + <PR | 94 |
| Brain glioma/astrocytoma | 80 | CR + PR + <PR | 88 |
| Prostate Carcinoma | 100 | CR + PR + <PR | 83 |
| Lymphoma/Leukemia | 80 | CR + PR + <PR | 72 |
| Head and Neck Cancer | 95 | CR + PR + <PR | 76 |
| Renal and Bladder Cancer | 90 | CR + PR + <PR | 92 |
| Melanoma | 95 | CR + PR + <PR | 86 |
| Neuroblastoma | 82 | CR + PR + <PR | 87 |
| Uterine/Cervical | 100 | CR + PR + <PR | 71 |

Toxicity consists of chills, fever, fatigue, nausea, vomiting, anorexia, tachycardia, hypotension.
The incidence of side effects (as % of total treatments) are as follows:
chills-5%;
fever-7%;
nausea-4%;
tachycardia-5%;
vomiting-5%;
hypotension-3%;
diarrhea-1%;
fatigue-8%.

1-8. (canceled)

9. A method of treating a subject with cancer comprising the steps of:
(i) contacting tumor associated antigens from a tumor bearing subject expressing MHCII HLA-DQ8 on said host myeloid cells wherein non-covalent complexes consisting of said tumor associated antigen and said MHCII HLA-DQ8 are formed:
(ii) contacting said complexes consisting of said tumor associated antigen and said MHCII HLA-DQ8 with CD4+ and CD8+ T cells that prime said CD4+ and said CD8 T cells for an anti-tumor response
(iii) administering to said subject parenterally by infusion or injection a composition consisting of wild type staphylococcal enterotoxin G and staphylococcal enterotoxin I; and
(iv) contacting said wild type staphylococcal enterotoxin G and staphylococcal enterotoxin I with endogenous HLA-DQ8 expressed on the surface of endogenous, autologous cells, wherein a non-covalent complex consisting of HLA-DQ8 with said staphylococcal enterotoxin G or with staphylococcal enterotoxin I is formed; and
(v) contacting said CD4+ and said CD8+ T cell of said subject with said complexes consisting of staphylococcal enterotoxin G or staphylococcal enterotoxin I bound to said HLA-DQ8 bound to said endogenous, autologous cells to produce T effector cells and T memory cells; and
(vi) thereby stimulating said CD4+ and said CD8+ T cells primed by said tumor associated antigen to induce a tumoricidal effect in said subject.

10. The method of claim 1, wherein following the administration of said staphylococcal enterotoxins G or I to the tumor site said staphylococcal enterotoxins G or I induce chemoattractants that recruit tumor reactive T effector cells to said tumor site that produce a tumoricidal response.

11. The method of claim 2 wherein said chemoattractants induced by said staphylococcal enterotoxin G or I at said tumor site consist of CCL2, CCL3, CCL5, CXCL9, CXCL10, CXCL9, CXCL10

12. A method of claim 1 wherein said staphylococcal enterotoxin G and said staphylococcal enterotoxin I molecules are selected from the group consisting of:
(i) a native staphylococcal enterotoxin G and staphylococcal enterotoxin I;
(ii) a biologically active homologue or fragment of a native staphylococcal enterotoxin which homologue or fragment:
(a) has the biological activity of stimulating T cell mitogenesis via a T cell receptor vβeta region of the T cell receptor; and
(b) has sequence homology characterized as a z value exceeding 13 when the sequence of the homologue or said fragment is compared to the sequence of a native staphylococcal enterotoxin determined by FASTA analysis using gap penalties of −12 and −2, Blosum 50 matrix and Swiss-PROT or PIR database.
